(19) United States
(12) Patent Application Publication
(10) Pub. No.: US 2011/0034534 A1
(43) Pub. Date: Feb. 10, 2011

(54) siRNA compounds and methods of use thereof

(76) Inventors: Elena Feinstein, Rehovot (IL); Hagar Kalinski, Rishon-le-Zion (IL); Igor Mett, Rehovot (IL); Evgenia Alpert, Jerusalem (IL)

Correspondence Address: COOPER & DUNHAM, LLP, 30 Rockefeller Plaza, 20th Floor, NEW YORK, NY, 10112, US

(21) Appl. No.: 12/804,255

(22) Filed: Jul. 15, 2010

Related U.S. Application Data
(63) Continuation of application No. PCT/IL2009/000053, filed on Jan. 14, 2009.
(60) Provisional application No. 61/011,337, filed on Jan. 15, 2008.

(51) Int. Cl. A61K 31/713; C07H 21/02; A61P 9/00; A61P 11/00; A61P 13/12; A61P 25/28; A61P 27/16; A61P 27/02

(52) U.S. Cl. 514/44 A; 536/24.5

(57) ABSTRACT

The present invention relates to compounds, pharmaceutical compositions comprising same and methods of use thereof for the inhibition of certain genes, including SOX9, ASPP1, CTSD, CAPNS1, FAS and FAS ligand. The compounds and compositions are useful in the treatment of subjects suffering from diseases or conditions and or symptoms associated with diseases or conditions in which gene expression has adverse consequences.

This application is a continuation of PCT Application No. PCT/IL2009/000053, filed Jan. 14, 2009 designating the United States and claims the benefit of U.S. provisional application No. 61/011,337, filed Jan. 15, 2008, the contents of both of which are hereby incorporated by reference in their entireties into this application.

This application incorporates-by-reference nucleotide and/or amino acid sequences which are present in the file named “189-PCT1-US1.ST25.txt,” which is 7.49 megabytes in size, was created on Jul. 13, 2010, in the IBM-PCT machine format, having an operating system compatibility with MS-Windows, and are contained on two identical duplicate compact discs labeled COPY 1 and COPY 2 submitted herewith.

Throughout this application various patents and publications are cited. The disclosures of these documents in their entireties are hereby incorporated by reference into this application to more fully describe the state of the art to which this invention pertains.

### FIELD OF THE INVENTION

The present invention relates to compounds, pharmaceutical compositions comprising same and methods of use thereof for the inhibition of certain genes, including SOX9, ASPP1, CTSD, CAPNS1, FAS and FAS ligand. The compounds and compositions are thus useful in the treatment of subjects suffering from diseases or conditions and or symptoms associated with such diseases or conditions in which gene expression has adverse consequences. In particular embodiments, the invention provides chemically modified siRNA oligonucleotides, compositions comprising same and methods of use thereof in the treatment of a neurodegenerative disease or disorder including spinal cord injury, Alzheimer's disease (AD) and Amyotrophic Lateral Sclerosis (ALS); acute renal failure (ARF); hearing loss; an ophthalmic disease including glaucoma and ischemic optic neuropathy (ION); a respiratory disease including acute respiratory distress syndrome (ARDS), chronic obstructive pulmonary disease (COPD) and other acute lung and respiratory injuries; injury (e.g. ischemia-reperfusion injury) in organ transplant including lung, kidney, bone marrow, heart, pancreas, cornea or liver transplantation; nephrotoxicity, pressure sores, dry eye syndrome or oral mucositis.

### BACKGROUND OF THE INVENTION

### siRNAs and RNA Interference

RNA interference (RNAi) is a phenomenon involving double-stranded (ds) RNA-dependent gene-specific posttranscriptional silencing. Initial attempts to study this phenomenon and to manipulate mammalian cells experimentally were frustrated by an active, non-specific antiviral defense mechanism which was activated in response to long dsRNA molecules (Gil et al., Apoptosis, 2000. 5:107-114). Later, it was discovered that synthetic duplexes of 21 nucleotide RNAs could mediate gene specific RNAi in mammalian cells, without stimulating the generic antiviral defense mechanisms Elbashir et al. Nature 2001, 411:494-498 and Caplen et al. PNAS 2001, 98:9742-9747). As a result, small interfering RNAs (siRNAs), which are short double-stranded RNAs, have been widely used to inhibit gene expression and understand gene function.

RNA interference (RNAi) is mediated by small interfering RNAs (siRNAs) (Fire et al, Nature 1998, 391:806) or microRNAs (miRNAs) (Ambros V. Nature 2004, 431:350-355); and Bartel D P. Cell. 2004 116(2):281-97). The corresponding process is commonly referred to as specific post-transcriptional gene silencing when observed in plants and as quelling when observed in fungi.

An siRNA is a double-stranded RNA which down-regulates or silences (i.e. fully or partially inhibits) the expression of an endogenous or exogenous gene/mRNA. RNA interference is based on the ability of certain dsRNA species to enter a specific protein complex, where they are then targeted to complementary cellular RNAs and specifically degrades them. Thus, the RNA interference response features an endonuclease complex containing an siRNA, commonly referred to as an RNA-induced silencing complex (RISC), which mediates cleavage of single-stranded RNA having a sequence complementary to the antisense strand of the siRNA duplex. Cleavage of the target RNA may take place in the middle of the region complementary to the antisense strand of the siRNA duplex (Elbashir, et al., Genes Dev., 2001, 15:188). In more detail, longer dsRNAs are digested into short (17-29 bp) dsRNA fragments (also referred to as short inhibitory RNAs or “siRNAs”) by type III RNAses (DICER, DROSHA, etc., (see Bernstein et al., Nature, 2001, 409:363-6 and Lee et al., Nature, 2003, 425:415-9). The RISC protein complex recognizes these fragments and complementary mRNA. The whole process is culminated by endonuclease cleavage of target mRNA (McManus and Sharp, Nature Rev Genet, 2002, 3:737-47; Paddison and Hannon, Curr Opin Mol Ther. 2003, 5(3): 217-24). (For additional information on these terms and proposed mechanisms, see for example, Bernstein, et al., RNA. 2001, 7(11):1509-21; Nishikura, Cell. 2001, 107(4):415-8 and PCT Publication No. WO 01/36646).

Studies have revealed that siRNA can be effective in vivo in both mammals and humans. Specifically, Bitko et al., showed that specific siRNAs directed against the respiratory syncytial virus (RSV) nucleocapsid N gene are effective in treating mice when administered intranasally (Nat. Med. 2005, 11(1):50-55). For reviews of therapeutic applications of siRNAs see for example Barik (Mol. Med 2005, 83: 764-773) and Chakraborty (Current Drug Targets 2007 8(3):469-82). In addition, clinical studies with short siRNAs that target the VEGFR1 receptor in order to treat age-related macular degeneration (AMD) have been conducted in human patients (Kaiser, Am J Ophthalmol. 2006 142(4):660-8). Further information on the use of siRNA as therapeutic agents may be found in Durcan, 2008. Mol. Pharma. 5(4):559-566; Kim and Rossi, 2008. BioTechniques 44:613-616; Grimm and Kay, 2007, JCI, 117(12):3633-41.

Chemically Modified siRNA

The selection and synthesis of siRNA corresponding to known genes has been widely reported; (see for example Ui-Tei et al., 2006. J Biomed Biotechnol.; 2006:65052; Chalk et al., 2004. BBRC. 319(1): 264-74; Sioud & Leirdal, 2004. Met. Mol Biol.; 252:457-69; Levenkova et al., 2004, Bioinform. 20(3):430-2; Ui-Tei et al., 2004. NAR 32(3):936-48).

For examples of the use of, and production of, modified siRNA see for example Braasch et al., 2003. Biochem., 42(26):7967-75; Chiu et al., 2003, RNA, 9(9):1034-48; PCT publications WO 2004/015107 (atugen AG) and WO 02/44321 (Tuschl et al). U.S. Pat. Nos. 5,898,031 and 6,107,094 teach chemically modified oligomers. U.S. Pat. No. 7,452,987 relates to oligomeric compounds having alternating unmodified and 2′ sugar modified ribonucleotides. US patent publication 2005/0042647 describes dsRNA compounds having chemically modified internucleoside linkages.

The inclusion of a 5′-phosphate moiety was shown to enhance activity of siRNAs in Drosophila embryos (Boutla, et al., 2001, Curr. Biol. 11:1776-1780) and is required for siRNA function in human HeLa cells (Schwarz et al., 2002, Mol. Cell, 10:537-548).

Amarzguoui et al., (2003, NAR, 31(2):589-595) showed that siRNA activity depended on the positioning of the 2′-O-methyl modifications. Holen et al (2003, NAR, 31(9):2401-2407) report that an siRNA having small numbers of 2′-O-methyl modified nucleosides showed good activity compared to wild type but that the activity decreased as the numbers of 2′-O-methyl modified nucleosides was increased. Chiu and Rana (2003, RNA, 9:1034-1048) teach that incorporation of 2′-O-methyl modified nucleosides in the sense or antisense strand (fully modified strands) severely reduced siRNA activity relative to unmodified siRNA. The placement of a 2′-O-methyl group at the 5′-terminus on the antisense strand was reported to severely limit activity whereas placement at the 3′-terminus of the antisense and at both termini of the sense strand was tolerated (Czauderna et al., 2003, NAR, 31(11), 2705-2716).

PCT Patent Publication Nos. PCT/IL2008/000248 and PCT/IL2008/001197 assigned to the assignee of the present invention disclose motifs useful in the preparation of chemically modified siRNA compounds.

Stable and active siRNA compounds useful in treating the above mentioned diseases and disorders would be of great therapeutic value.

### SUMMARY OF THE INVENTION

The present invention provides inhibitors of a target gene selected from the group consisting of SOX9, ASPP1, CTSD, CAPNS1, FAS and FAS ligand (See Table 1, infra, for genes' details). In various embodiments the inhibitor is selected from the group consisting of unmodified or chemically modified siRNA, shRNA, an aptamer, an antisense molecule, miRNA, and a ribozyme. In the presently preferred embodiments the inhibitor is chemically modified siRNA.

In one aspect the present invention provides novel double stranded oligoribonucleotide compounds that inhibit expression of a gene selected from the group consisting of SOX9, ASPP1, CTSD, CAPNS1, FAS and FAS ligand. In particular embodiments the double stranded oligoribonucleotide compounds are chemically modified siRNA.

The present invention provides a compound having the structure:

5′(N)ₓ—Z3′ (antisense strand)

3′Z′—(N′)_{y}-z″5′ (sense strand)

wherein each of N and N′ is a ribonucleotide which may be unmodified or modified, or an unconventional moiety;
wherein each of (N)x and (N′)y is an oligonucleotide in which each consecutive N or N′ is joined to the next N or N′ by a covalent bond;
wherein Z and Z′ may be present or absent, but if present is independently 1-5 consecutive nucleotides covalently attached at the 3′ terminus of the strand in which it is present;
wherein z″ may be present or absent, but if present is a capping moiety covalently attached at the 5′ terminus of (N′)y;
each of x and y is independently an integer between 18 and 40;
wherein the sequence of (N′)y is substantially complementary to the sequence of (N)x; and
wherein (N)x comprises an antisense sequence to an mRNA set forth in any one of SEQ ID NOS: 41025-41038.

In some embodiments (N)x is present in any of Tables A-AQ.

In some embodiments the covalent bond joining each consecutive N or N′ is a phosphodiester bond. In various embodiments all the covalent bonds are phosphodiester bonds.

In various embodiments the compound comprises ribonucleotides wherein x=y and each of x and y is 19, 20, 21, 22 or 23. In some embodiments x=y=23. In other embodiments x=y=19.

In some embodiments the compound is blunt ended, for example wherein both Z and Z′ are absent. In an alternative embodiment, the compound comprises at least one 3′ overhang, wherein at least one of Z or Z′ is present. Z and Z′ can independently comprise one or more covalently linked modified or non-modified nucleotides, for example inverted dT or dA; dT, LNA, mirror nucleotide and the like. In some embodiments each of Z and Z′ are independently selected from dT and dTdT.

In some embodiments each of (N)x and (N′)y consist of unmodified nucleotides.

In some embodiments N or N′ comprises a modification in the sugar residue of one or more ribonucleotides. In other embodiments the compound comprises at least one ribonucleotide modified in the sugar residue. In some embodiments the compound comprises a modification at the 2′ position of the sugar residue. In some embodiments the modification in the 2′ position comprises the presence of an amino, a fluoro, an alkoxy or an alkyl moiety. In certain embodiments the 2′ modification comprises a methoxy moiety (also known as 2′-O-methyl; 2′-O-Me; 2′-O—CH₃). In some embodiments in each of (N)x and (N′)y the ribonucleotides alternate between modified ribonucleotides and unmodified ribonucleotides each modified ribonucleotide being modified so as to have a 2′-O-methyl on its sugar and the ribonucleotide located at the middle position of (N)x being unmodified and the ribonucleotide located at the middle position of (N′)y being modified.

In some embodiments the siRNA compound comprises modified alternating ribonucleotides in one or both of the antisense and the sense strands. In other embodiments the compound comprises modified alternating ribonucleotides in the antisense strand (N)x only. In certain embodiments the middle ribonucleotide of the antisense strand is not modified; e.g. ribonucleotide in position 10 in a 19-mer strand or position 12 in a 23-mer strand.

In additional embodiments the compound comprises modified ribonucleotides in alternating positions wherein each N at the 5′ and 3′ termini of (N)ₓ are modified in their sugar residues, and each N′ at the 5′ and 3′ termini of (N′)_{y} are unmodified in their sugar residues. In some embodiments, neither (N)ₓ nor (N′)_{y} are phosphorylated at the 3′ and 5′ termini. In other embodiments either or both (N)ₓ and (N′)_{y} are phosphorylated at the 3′ termini.

In some embodiments (N)x comprises modified and unmodified ribonucleotides, each modified ribonucleotide having a 2′-O-methyl on its sugar, wherein N at the 3′ terminus of (N)x is a modified ribonucleotide, (N)x comprises at least five alternating modified ribonucleotides beginning at the 3′ end and at least nine modified ribonucleotides in total and each remaining N is an unmodified ribonucleotide. In some embodiments, (N)x comprises 2′O Me modified ribonucleotides at positions 2, 4, 6, 8, 11, 13, 15, 17 and 19. In other embodiments (N)x comprises 2′O Me modified ribonucleotides at positions 1, 3, 5, 7, 9, 11, 13, 15, 17 and 19.

In some embodiments the unconventional moiety is selected from a mirror nucleotide and a nucleotide joined to an adjacent nucleotide by a 2′-5′ internucleotide phosphate bond. In some embodiments the mirror nucleotide is selected from an L-ribonucleotide (L-RNA) and an L-deoxyribonucleotide (L-DNA). In some embodiments (N′)y comprises at least one unconventional moiety.

In one embodiment of the above structure, the compound comprises at least one mirror nucleotide at one or both termini in (N′)y. In various embodiments the compound comprises two consecutive mirror nucleotides, one at the 3′ penultimate position and one at the 3′ terminus in (N′)y. In one preferred embodiment x=y=19 and (N′)y comprises an L-DNA at position 18.

In some embodiments x=y=19 and (N′)y, consists of unmodified ribonucleotides at positions 1-17 and 19 and one L-DNA at the 3′ penultimate position (position 18). In other embodiments x=y=19 and (N′)y consists of unmodified ribonucleotides at position 1-16 and 19 and two consecutive L-DNA at the 3′ penultimate position (positions 17 and 18).

In another embodiment of the above structure, (N′)y further comprises one or more nucleotides containing an intra-sugar bridge at one or both termini.

In one embodiment the present invention provides a compound having the structure (Structure 1):

5′(N)x—-Z3′ antisense strand (1)

3′Z′—(N′)y5′ sense strand

wherein each of N and N′ is a nucleotide selected from an unmodified ribonucleotide, a modified ribonucleotide, an unmodified deoxyribonucleotide and a modified deoxyribonucleotide;
wherein each of (N)x and (N′)y is an oligomer in which each consecutive nucleotide is joined to the next nucleotide by a covalent bond and each of x and y is an integer between 18 and 40;
wherein in (N)x the nucleotides alternate between modified ribonucleotides and unmodified ribonucleotides, each modified ribonucleotide being modified so as to have a 2′-O-methyl on its sugar and the ribonucleotide located at the middle of (N)x being unmodified;
wherein (N′)y comprises unmodified ribonucleotides in which three nucleotides at the 3′ terminus of (N′)y are joined by two 2′-5′ phosphodiester bonds; and
wherein (N)ₓ and (N′)_{y} together are a pair of oligonucleotides present in any of Tables A-AQ.

In another embodiment the present invention provides a compound having the structure (Structure II):

5′(N)x-Z3′ antisense strand (II)

3′Z′—(N′)y5′ sense strand

wherein each of N and N′ is a nucleotide selected from an unmodified ribonucleotide, a modified ribonucleotide, an unmodified deoxyribonucleotide and a modified deoxyribonucleotide;
wherein each of (N)x and (N′)y is an oligomer in which each consecutive nucleotide is joined to the next nucleotide by a covalent bond and each of x and y is an integer between 18 and 40;
wherein (N)x comprises unmodified ribonucleotides in which three consecutive nucleotides at the 3′ terminus are joined together by two 2′-5′ phosphodiester bonds; and (N′)y comprises unmodified ribonucleotides in which four consecutive nucleotides at the 5′ terminus are joined together by three 2′-5′ phosphodiester bonds; and
wherein (N)ₓ and (N′)_{y} together are a pair of oligonucleotides present in any of Tables A-AQ.

In another aspect the present invention provides a compound having the structure (Structure III):

5′(N)x-Z3′ antisense strand (III)

3′Z′—(N′)y5′ sense strand

wherein each of N and N′ is a nucleotide selected from an unmodified ribonucleotide, a modified ribonucleotide, an unmodified deoxyribonucleotide and a modified deoxyribonucleotide;
wherein each of (N)x and (N′)y is an oligomer in which each consecutive nucleotide is joined to the next nucleotide by a covalent bond and each of x and y is an integer between 18 and 40;
wherein (N′)y comprises unmodified ribonucleotides in which two consecutive nucleotides at the 3′ terminus are replaced by two consecutive mirror deoxyribonucleotides; and (N)x comprises unmodified ribonucleotides in which one nucleotide at the 3′ terminus is replaced by a mirror deoxyribonucleotide; and
wherein (N)ₓ and (N′)_{y} together are a pair of oligonucleotides present in any of Tables A-AQ.

In a second aspect the present invention provides pharmaceutical compositions comprising one or more such compounds according to the present invention; and a pharmaceutically acceptable excipient.

In a third aspect the present invention relates to a method for treating or preventing the incidence or severity of a disease or condition in a subject in need thereof wherein the disease or condition and/or symptoms associated therewith is selected from the group consisting of a neurodegenerative disease or disorder including spinal cord injury, Alzheimer's disease (AD) and Amyotrophic Lateral Sclerosis (ALS); acute renal failure (ARF); hearing loss; an ophthalmic disease including glaucoma and ischemic optic neuropathy (ION); a respiratory disease including acute respiratory distress syndrome (ARDS), chronic obstructive pulmonary disease (COPD) and other acute lung and respiratory injuries; injury (e.g. ischemia-reperfusion injury) in organ transplant including lung, kidney, bone marrow, heart, pancreas, cornea or liver transplantation; nephrotoxicity, pressure sores, dry eye syndrome or oral mucositis.

Such methods involve administering to a mammal in need of such treatment a prophylactically or therapeutically effective amount of one or more such compounds which inhibit or reduce expression or activity of at least one such gene. In another aspect, the present invention relates to a method for the treatment of a subject in need of treatment for a disease or disorder or symptoms associated with the disease or disorder, associated with the expression of a gene selected from SOX9, ASPP1, CTSD, CAPNS1, FAS and FAS ligand, comprising administering to the subject an amount of an siRNA which reduces or inhibits expression of at least one of those genes.

In one embodiment, the present invention provides a method of treating a neurodegenerative disease or disorder in a subject in need thereof, comprising administering to the subject an siRNA compound which inhibits expression of SOX9, CTSD or CAPNS1 in an amount effective to treat the neurodegenerative disease or disorder. In some embodiments the neurodegenerative disease or disorder is selected from spinal cord injury, Alzheimer's disease and amyotrophic lateral sclerosis.

In another embodiment, the present invention provides a method of treating an ophthalmic disease in a subject in need thereof, comprising administering to the subject an siRNA compound which inhibits expression of SOX9, ASPP1, CTSD or CAPNS1 in an amount effective to treat the ophthalmic disease. In some embodiments the ophthalmic disease is selected from glaucoma, ION and AMD.

In another embodiment, the present invention provides a method of treating dry eye in a subject in need thereof, comprising administering to the subject an oligonucleotide which inhibits expression of FAS and FAS ligand, in an amount effective to treat the dry eye.

Lists of 19-mer, 21-mer and 23-mer sense and corresponding antisense oligonucleotides useful in the preparation of siRNA compounds are provided in Tables A-AQ, set forth in SEQ ID NOS:1-41,024.

### DETAILED DESCRIPTION OF THE INVENTION

The present invention relates generally to compounds which down-regulate expression of various genes, particularly to novel chemically modified small interfering RNA oligonucleotides (siRNAs), and to the use of these novel siRNAs in the treatment of various diseases and medical conditions. According to one aspect the present invention provides inhibitory oligonucleotide compounds comprising unmodified and modified nucleotides and or unconventional moieties. The compound comprises at least one modified nucleotide selected from the group consisting of a sugar modification, a base modification and an internucleotide linkage modification and may contain DNA, and modified nucleotides such as LNA (locked nucleic acid), ENA (ethylene-bridged nucleic acid, PNA (peptide nucleic acid), arabinoside, PACE, mirror nucleotide, or nucleotides with a 6 carbon sugar.

Particular diseases and conditions to be treated are a neurodegenerative disease or disorder including spinal cord injury, Alzheimer's disease (AD) and Amyotrophic Lateral Sclerosis (ALS); acute renal failure (ARF); hearing loss; an ophthalmic disease including glaucoma and ischemic optic neuropathy (ION); a respiratory disease including acute respiratory distress syndrome (ARDS), chronic obstructive pulmonary disease (COPD) and other acute lung and respiratory injuries; injury (e.g. ischemia-reperfusion injury) in organ transplant including lung, kidney, bone marrow, heart, pancreas, cornea or liver transplantation; nephrotoxicity, pressure sores, dry eye syndrome or oral mucositis.

Lists of preferred siRNA to be used in the present invention are provided in Tables A-AQ. For each gene there is a separate list of 19-mer, 21-mer and 23-mer sequences, which are prioritized based on their score in the proprietary algorithm as the best sequences for targeting the human gene expression. 21- or 23-mer siRNA sequences can also be generated by 5′ and/or 3′ extension of the 19-mer sequences disclosed herein. Such extension is preferably complementary to the corresponding mRNA sequence. Certain 23-mer oligomers were devised by this method where the order of the prioritization is the order of the corresponding 19-mer.

Methods, molecules and compositions which inhibit the genes of the invention are discussed herein at length, and any of said molecules and/or compositions may be beneficially employed in the treatment of a subject suffering from any of said conditions.

Tables A, E, I, L, O, S, W, AA, AD, AF, AJ, AN, set forth 19-mer oligomers. Tables B, F, J, M, P, T, X, AB, AG, AK, AO, set forth 21-mer oligomers. Tables C, D, G, H, K, N, Q, R, U, V, Y, Z, AC, AE, AH, AI, AL, AM, AP, AQ, set forth 23-mer oligomers.

Tables A-D and AN-AQ provide siRNA sense and antisense oligonucleotides to SOX9, set forth in SEQ ID NOS:1-4686 and SEQ ID NOS:37133-40972. Tables E-H and W-Z provide siRNA sense and antisense oligonucleotides to ASPP1, set forth in SEQ ID NOS 4687-10192 and SEQ ID NOS:24921-29288. Tables I-K and AA-AC provide siRNA sense and antisense oligonucleotides to CAPNS1 set forth in SEQ ID NO:10193-13428 AND SEQ ID NOS:29289-29718. Tables L-N and AD-AE provide siRNA sense and antisense oligonucleotides to CTSD set forth in SEQ ID NOS:13429-16016 and SEQ ID NOS:29719-30216 and 40973-41024. Tables O-R and AF-AI provide siRNA sense and antisense oligonucleotides to FAS set forth in SEQ ID NOS:16017-20608 and SEQ ID NOS:30217-33810. Tables S-V and AJ-AM provide siRNA sense and antisense oligonucleotides to FAS LIGAND set forth in SEQ ID NOS: 20609-24920 and SEQ ID NOS:33811-37132.

The siRNAs of the present invention possess structures and modifications which may increase activity, increase stability, and or minimize toxicity; the novel modifications of the siRNAs of the present invention can be beneficially applied to double stranded RNA useful in preventing or attenuating one or more of the target genes' expression.

Table 1, below, sets forth the gene identification number (gi) with an NCBI accession number for the respective mRNA sequences, the SEQ ID NO for the corresponding mRNA and polypeptide, and a description of the gene/protein function.

TABLE 1
Target genes of the present invention

| | | Certain |
|---|---|---|
| | Full name and Human Gene ID | preferred |
| Gene | Short description | indications |
| SOX9 | SRY (sex determining region Y)-box 9 (campomelic dysplasia, | ARF, ARDS, |
| | autosomal sex-reversal) gi\|37704387\|ref\|NM_000346.3\| | glaucoma, |
| | (mRNA is set forth in SEQ ID NO: 41025 polypeptide | ION, SCI, |
| | sequence is set forth in SEQ ID NO: 41039) | COPD, |
| | Description: SOX9 protein recognizes the sequence CCTTGAG | |
| | along with other members of the HMG-box class DNA-binding | |
| | proteins. It acts during chondrocyte differentiation and, with | |
| | steroidogenic factor 1, regulates transcription of the anti- | |
| | Muellerian hormone (AMH) gene. Deficiencies lead to the | |
| | skeletal malformation syndrome. | |
| ASPP1 | protein phosphatase 1, regulatory (inhibitor) subunit 13B | ARF, hearing |
| | (PPP1R13B) gi\|121114286\|ref\|NM_015316.2\| | loss, |
| | (mRNA is set forth in SEQ ID NO: 41026; polypeptide | glaucoma, |
| | sequence is set forth in SEQ ID NO: 41040) | ION, SCI, |
| | Description: This gene encodes a member of the ASPP | COPD, DR, |
| | (apoptosis-stimulating protein of p53) family of p53 interacting | ARDS, |
| | proteins. ASPP proteins are required for the induction of | |
| | apoptosis by p53-family proteins. They promote DNA binding | |
| | and transactivation of p53-family proteins on the promoters of | |
| | proapoptotic genes. | |
| CTSD | Cathepsin D gi\|23110949\|ref\|NM_001909.3\| | ARF, hearing |
| | (mRNA is set forth in SEQ ID NO: 41027 | loss, ARDS, |
| | polypeptide sequence is set forth in SEQ ID NO: 41041) | glaucoma, |
| | Cathepsin D is a lysosomal aspartyl protease composed of a | ION, SCI, |
| | dimer of disulfide-linked heavy and light chains, both produced | COPD, DR, |
| | from a single protein precursor. Transcription of this gene is | ND, |
| | initiated from several sites, including one which is a start site for | transplant |
| | an estrogen-regulated transcript. Mutations in this gene are | |
| | involved in the pathogenesis of several diseases, including breast | |
| | cancer and possibly Alzheimer disease. | |
| CAPNS1 | Calpain small subunit 1 | ARF, hearing |
| | gi\|51599152\|ref\|NM_001749.2\| Variant 1 (mRNA is set forth in | loss, ARDS, |
| | SEQ ID NO: 41028; polypeptide sequence is set forth in | glaucoma, |
| | SEQ ID NO: 41042) | ION, SCI, |
| | gi\|51599150\|ref\|NM_001003962.1\| variant 2 (mRNA is set forth | COPD, DR, |
| | in SEQ ID NO: 41029; polypeptide sequence is set forth in | ND (ALS, |
| | SEQ ID NO:41043) | AD, SCI) |
| | Description: Calpains are a ubiquitous, well-conserved family of | |
| | calcium-dependent, cysteine proteases. Calpain families have | |
| | been implicated in neurodegenerative processes, as their | |
| | activation can be triggered by calcium influx and oxidative stress. | |
| | Two transcript variants encoding the same protein have been | |
| | identified for this gene. | |
| FAS | (TNF receptor superfamily, member 6) | Dry eye |
| | gi\|23510419\|ref\|NM_000043.3\| variant 1 (mRNA is set forth in | ARF, hearing |
| | SEQ ID NO: 41030; polypeptide sequence is set forth in | loss, ARDS, |
| | SEQ ID NO: 41044) | glaucoma, |
| | gi\|23510422\|ref\|NM_152872.1\| variant 3 (mRNA is set forth in | SCI, COPD, |
| | SEQ ID NO: 41031; polypeptide sequence is set forth in | DR, ND, |
| | SEQ ID NO: 41045) | transplant |
| | gi\|23510420\|ref\|NM_152871.1\| variant 2 (mRNA is set forth in | |
| | SEQ ID NO: 41032; polypeptide sequence is set forth in | |
| | SEQ ID NO: 41046) | |
| | gi\|23510424\|ref\|NM_152873.1\| variant 4 (mRNA is set forth in | |
| | SEQ ID NO: 41033; polypeptide sequence is set forth in | |
| | SEQ ID NO: 41047) | |
| | gi\|23510426\|ref\|NM_152874.1\| variant 8 (mRNA is set forth in | |
| | SEQ ID NO: 41034; polypeptide sequence is set forth in | |
| | SEQ ID NO: 41048) | |
| | gi\|23510428\|ref\|NM_152875.1\| variant 5 (mRNA is set forth in | |
| | SEQ ID NO: 41035; polypeptide sequence is set forth in | |
| | SEQ ID NO: 41049) | |
| | gi\|23510433\|ref\|NM_152877.1\| variant 7 (mRNA is set forth in | |
| | SEQ ID NO: 41036; polypeptide sequence is set forth in | |
| | SEQ ID NO: 41050) | |
| | gi\|23510430\|ref\|NM_152876.1\| variant 6 (mRNA is set forth in | |
| | SEQ ID NO: 41037; polypeptide sequence is set forth in | |
| | SEQ ID NO: 41051) | |
| | Description: The protein encoded by this gene contains a death | |
| | domain and is a member of the TNF-receptor superfamily. It has | |
| | been shown to play a central role in the physiological regulation | |
| | of programmed cell death, and has been implicated in the | |
| | pathogenesis of various malignancies and diseases of the immune | |
| | system. The interaction of this receptor with its ligand allows the | |
| | formation of a death-inducing signaling complex that includes | |
| | Fas-associated death domain protein (FADD), caspase 8, and | |
| | caspase 10. The autoproteolytic processing of the caspases in the | |
| | complex triggers a downstream caspase cascade, and leads to | |
| | apoptosis. At least eight alternatively spliced transcript variants | |
| | encoding seven distinct isoforms have been described. The | |
| | isoforms lacking the transmembrane domain may negatively | |
| | regulate the apoptosis mediated by the full length isoform. | |
| FASLG | Fas ligand (TNF superfamily, member 6) | Dry eye |
| | gi\|4557328\|ref\|NM_300639.1\| (mRNA is set forth in | ARF, hearing |
| | SEQ ID NO: 41038; polypeptide sequence is set forth in | loss, ARDS, |
| | SEQ ID NO: 41052) | glaucoma, |
| | Description: The protein encoded by this gene is the | SCI, COPD, |
| | ligand for FAS. Both are transmembrane proteins. Interaction of | DR, ND, |
| | FAS with this ligand is critical in triggering apoptosis of some | transplant |
| | types of cells such as lymphocytes. Defects in this gene may be | |
| | related to some cases of systemic lupus erythematosus (SLE). | |

ND: neurodegenerative diseases and disorders; AD: Alzheimer's disease;
ALS: Amyotrophic Lateral Sclerosis; ARF: acute renal failure,
ARDS, acute respiratory distress syndrome, COPD chronic obstructive pulmonary disease,
DR: diabetic retinopathy; SCI spinal cord injury.

In non-limiting examples, siRNA to SOX9 is useful in treating, inter alia, a neurodegenerative disorder, in particular spinal cord injury and Alzheimer's disease; siRNA to ASPP1 is useful in treating, inter alia, hearing loss, acute renal failure, glaucoma, neurodegenerative disease; siRNA to Fas (CD95) or Fas ligand is useful in treating dry eye; siRNA to cathepsin D (CTSD) or to Calpain small subunit 1 (CAPNS1) is useful in treating hearing loss and neurodegenerative disorders including spinal cord injury, Alzheimer's disease and ALS.

### DEFINITIONS

For convenience certain terms employed in the specification, examples and claims are described herein.

It is to be noted that, as used herein, the singular forms “a”, “an” and “the” include plural forms unless the content clearly dictates otherwise.

Where aspects or embodiments of the invention are described in terms of Markush groups or other grouping of alternatives, those skilled in the art will recognize that the invention is also thereby described in terms of any individual member or subgroup of members of the group.

An “inhibitor” is a compound, which is capable of reducing (partially or fully) the expression of a gene or the activity of the product of such gene to an extent sufficient to achieve a desired biological or physiological effect. The term “inhibitor” as used herein refers to one or more of an oligonucleotide inhibitor, including siRNA, shRNA, synthetic shRNA; miRNA, antisense RNA and DNA and ribozymes.

A “siRNA inhibitor” is a compound which is capable of reducing the expression of a gene or the activity of the product of such gene to an extent sufficient to achieve a desired biological or physiological effect. The term “siRNA inhibitor” as used herein refers to one or more of a siRNA, shRNA, synthetic shRNA; miRNA. Inhibition may also be referred to as down-regulation or, for RNAi, silencing

The term “inhibit” as used herein refers to reducing the expression of a gene or the activity of the product of such gene to an extent sufficient to achieve a desired biological or physiological effect. Inhibition may be complete or partial.

As used herein, the term “inhibition” of a target gene means inhibition of the gene expression (transcription or translation) or polypeptide activity of a gene selected from the group SOX9, ASPP1, CAPNS1, CTSD, FAS or Fas ligand or an SNP (single nucleotide polymorphism) or other variants thereof. The gi number for the mRNA of each target gene is set forth in Table 1. The polynucleotide sequence of the target mRNA sequence, refers to the mRNA sequences set forth in SEQ ID NO:41025-41038, or any homologous sequences thereof preferably having at least 70% identity, more preferably 80% identity, even more preferably 90% or 95% identity to any one of mRNA set forth in SEQ ID NO:41025-41038. Therefore, polynucleotide sequences derived from any one of SEQ ID NO:41025-41038 which have undergone mutations, alterations or modifications as described herein are encompassed in the present invention. The terms “mRNA polynucleotide sequence” and “mRNA” are used interchangeably.

As used herein, the terms “polynucleotide” and “nucleic acid” may be used interchangeably and refer to nucleotide sequences comprising deoxyribonucleic acid (DNA), and ribonucleic acid (RNA). The terms should also be understood to include, as equivalents, analogs of either RNA or DNA made from nucleotide analogs. Throughout this application, mRNA sequences are set forth as representing the corresponding genes.

“Oligonucleotide” or “oligomer” refers to a deoxyribonucleotide or ribonucleotide sequence from about 2 to about 50 nucleotides. Each DNA or RNA nucleotide may be independently natural or synthetic, and or modified or unmodified. Modifications include changes to the sugar moiety, the base moiety and or the linkages between nucleotides in the oligonucleotide. The compounds of the present invention encompass molecules comprising deoxyribonucleotides, ribonucleotides, modified deoxyribonucleotides, modified ribonucleotides and combinations thereof.

“Nucleotide” is meant to encompass deoxyribonucleotides and ribonucleotides, which may be natural or synthetic, and or modified or unmodified. Modifications include changes to the sugar moiety, the base moiety and or the linkages between ribonucleotides in the oligoribonucleotide. As used herein, the term “ribonucleotide” encompasses natural and synthetic, unmodified and modified ribonucleotides. Modifications include changes to the sugar moiety, to the base moiety and/or to the linkages between ribonucleotides in the oligonucleotide.

According to some embodiments the present invention provides inhibitory oligonucleotide compounds comprising unmodified and modified nucleotides and or unconventional moieties. The compound comprises at least one modified nucleotide selected from the group consisting of a sugar modification, a base modification and an internucleotide linkage modification and may contain DNA, and modified nucleotides such as LNA (locked nucleic acid), ENA (ethylene-bridged nucleic acid. PNA (peptide nucleic acid), arabinoside, PACE, mirror nucleotide, or nucleotides with a 6 carbon sugar.

All analogs of, or modifications to, a nucleotide/oligonucleotide may be employed with the present invention, provided that said analog or modification does not substantially adversely affect the function of the nucleotide/oligonucleotide. Acceptable modifications include modifications of the sugar moiety, modifications of the base moiety, modifications in the internucleotide linkages and combinations thereof.

In one embodiment the compound comprises a 2′ modification on the sugar moiety of at least one ribonucleotide (“2′ sugar modification”). In certain embodiments the compound comprises 2′O-alkyl or 2′-fluoro or 2′O-allyl or any other 2′ modification, optionally on alternate positions. Other stabilizing modifications are also possible (e.g. terminal modifications). In some embodiments a preferred 2′O-alkyl is 2′O-methyl (methoxy) sugar modification.

In some embodiments the backbone of the oligonucleotides is modified and comprises phosphate-D-ribose entities but may also contain thiophosphate-D-ribose entities, triester, thioate, 2′-5′ bridged backbone (also may be referred to as 5′-2′), PACE and the like.

As used herein, the terms “non-pairing nucleotide analog” means a nucleotide analog which comprises a non-base pairing moiety including but not limited to: 6 des amino adenosine (Nebularine), 4-Me-indole, 3-nitropyrrole, 5-nitroindole, Ds, Pa, N3-Me ribo U, N3-Me riboT, N3-Me dC, N3-Me-dT, N1-Me-dG, N1-Me-dA, N3-ethyl-dC, N3-Me dC. In some embodiments the non-base pairing nucleotide analog is a ribonucleotide. In other embodiments it is a deoxyribonucleotide.

Other modifications include terminal modifications on the 5′ and/or 3′ part of the oligonucleotides and are also known as capping moieties. Such terminal modifications are selected from a nucleotide, a modified nucleotide, a lipid, a peptide, and a sugar.

A sugar modification includes a modification on the 2′ moiety of the sugar residue and encompasses amino, fluoro, alkoxy e.g. methoxy, alkyl, amino, fluoro, chloro, bromo, CN, CF, imidazole, carboxylate, thioate, C₁ to C₁₀ lower alkyl, substituted lower alkyl, alkaryl or aralkyl, OCF₃, OCN, O-, S-, or N-alkyl; O-, S, or N-alkenyl; SOCH₃; SO₂CH₃; ONO₂; NO₂, N₃; heterocycloalkyl; heterocycloalkaryl; aminoalkylamino; polyalkylamino or substituted silyl, as, among others, described in European patents EP 0 586 520 B1 or EP 0 618 925 B1.

The present invention provides methods and compositions for inhibiting expression of the target gene in vivo. In general, the method includes administering oligoribonucleotides, in particular small interfering RNAs (i.e. siRNAs) or a nucleic acid material that can produce siRNA in a cell, that target an mRNA transcribed from the target gene in an amount sufficient to down-regulate expression of a target gene by an RNA interference mechanism. In particular, the subject method can be used to inhibit expression of the target gene for treatment of a disease. In accordance with the present invention, the siRNA molecules or inhibitors of the target gene are used as drugs to treat various pathologies.

siRNA Oligoribonucleotides

Tables A-AQ provide nucleic acid sequences of sense and corresponding antisense oligonucleotides, useful in preparing unmodified and chemically siRNA compounds.

The selection and synthesis of siRNA corresponding to known genes has been widely reported; see for example Ui-Tei et al., J Biomed Biotechnol. 2006; 65052; Chalk et al., BBRC. 2004, 319(1):264-74; Sioud and Leirdal, Met. Mol Biol.; 2004, 252:457-69; Levenkova et al., Bioinform. 2004, 20(3):430-2; Ui-Tei et al., NAR 2004, 32(3):936-48. For examples of the use of, and production of, modified siRNA see Braasch et al., Biochem., 2003, 42(26):7967-75; Chiu et al., RNA, 2003, 9(9):1034-48; PCT publications WO 2004/015107 (atugen); WO 02/44321 (Tuschl et al), and U.S. Pat. Nos. 5,898,031 and 6,107,094.

Several groups have described the development of DNA-based vectors capable of generating siRNA within cells. The method generally involves transcription of short hairpin RNAs that are efficiently processed to form siRNAs within cells (Paddison et al. PNAS USA 2002, 99:1443-1448; Paddison et al. Genes & Dev 2002, 16:948-958; Sui et al. PNAS USA 2002, 8:5515-5520; and Brummelkamp et al. Science 2002, 296:550-553).

These reports describe methods of generating siRNAs capable of specifically targeting numerous endogenously and exogenously expressed genes.

The present invention provides double-stranded oligoribonucleotides (eg. siRNAs), which down-regulate the expression of the target genes according to the present invention. An siRNA of the invention is a duplex oligoribonucleotide in which the sense strand is derived from the mRNA sequence of the target gene, and the antisense strand is complementary to the sense strand. In general, some deviation from the target mRNA sequence is tolerated without compromising the siRNA activity (see e.g. Czauderna et al., 2003, NAR 31(11), 2705-2716). An siRNA of the invention inhibits gene expression on a post-transcriptional level with or without destroying the mRNA. Without being bound by theory, siRNA may target the mRNA for specific cleavage and degradation and/or may inhibit translation from the targeted message.

According to the present invention the siRNA compounds are chemically and or structurally modified according to one of the following modifications set forth in Structures (A)-(P) or as tandem siRNA or RNAstar.

In one aspect the present invention provides a compound set forth as Structure (A):

5′(N)ₓ—Z3′ (antisense strand) (A)

3′Z′—(N′)_{y}5′ (sense strand)

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein each of x and y is an integer between 18 and 40;
wherein each of Z and Z′ may be present or absent, but if present is 1-5 consecutive nucleotides covalently attached at the 3′ terminus of the strand in which it is present;
wherein the sequence of (N′)_{y} is a sequence substantially complementary to (N)x; and
wherein the sequence of (N)ₓ comprises an antisense sequence substantially identical to an antisense sequence set forth in any one of Tables A-AQ.

In certain embodiments the present invention provides a compound having structure (B)

5′(N)x3′ antisense strand (B)

3′(N′)y5′ sense strand

wherein each of (N)ₓ and (N′)_{y} is an oligomer in which each consecutive N or N′ is an unmodified ribonucleotide or a modified ribonucleotide joined to the next N or N′ by a covalent bond;
wherein each of x and y=19, 21 or 23 and (N)ₓ and (N′)_{y} are fully complementary wherein alternating ribonucleotides in each of (N)ₓ and (N′)_{y} are modified to result in a 2′-O-methyl modification in the sugar residue of the ribonucleotides;
wherein the sequence of (N′)_{y} is a sequence substantially complementary to (N)x; and
wherein the sequence of (N)ₓ comprises an antisense sequence substantially identical to an antisense sequence set forth in any one of Tables A-AQ.

In some embodiments each of (N)ₓ and (N′)_{y} is independently phosphorylated or non-phosphorylated at the 3′ and 5′ termini.

In certain embodiments wherein each of x and y=19 or 23, each N at the 5′ and 3′ termini of (N)ₓ is modified; and

each N′ at the 5′ and 3′ termini of (N′)_{y} is unmodified.

In certain embodiments wherein each of x and y=21, each N at the 5′ and 3′ termini of (N)ₓ is unmodified; and

each N′ at the 5′ and 3′ termini of (N′)_{y} is modified.

In particular embodiments, when x and y=19, the siRNA is modified such that a 2′-O-methyl (2′-OMe) group is present on the first, third, fifth, seventh, ninth, eleventh, thirteenth, fifteenth, seventeenth and nineteenth nucleotide of the antisense strand (N)ₓ, and whereby the very same modification, i.e. a 2′-OMe group, is present at the second, fourth, sixth, eighth, tenth, twelfth, fourteenth, sixteenth and eighteenth nucleotide of the sense strand (N′)_{y}. In various embodiments these particular siRNA compounds are blunt ended at both termini.

In some embodiments, the present invention provides a compound having Structure (C):

5′(N)x-Z3′ antisense strand (C)

3′Z′—(N′)y5′ sense strand

wherein each of N and N′ is a nucleotide independently selected from an unmodified ribonucleotide, a modified ribonucleotide, an unmodified deoxyribonucleotide and a modified deoxyribonucleotide;
wherein each of (N)x and (N′)y is an oligomer in which each consecutive nucleotide is joined to the next nucleotide by a covalent bond and each of x and y is an integer between 18 and 40;
wherein in (N)x the nucleotides are unmodified or (N)x comprises alternating modified ribonucleotides and unmodified ribonucleotides; each modified ribonucleotide being modified so as to have a 2′-O-methyl on its sugar and the ribonucleotide located at the middle position of (N)x being modified or unmodified preferably unmodified;
wherein (N′)y comprises unmodified ribonucleotides further comprising one modified nucleotide at a terminal or penultimate position, wherein the modified nucleotide is selected from the group consisting of a mirror nucleotide, a bicyclic nucleotide, a 2′-sugar modified nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein if more than one nucleotide is modified in (N′)y, the modified nucleotides may be consecutive;
wherein each of Z and Z′ may be present or absent, but if present is 1-5 deoxyribonucleotides covalently attached at the 3′ terminus of any oligomer to which it is attached;
wherein the sequence of (N′)_{y} comprises a sequence substantially complementary to (N)x; and wherein (N)ₓ comprises an antisense sequence substantially identical to an antisense sequence set forth in any one of Tables A-AQ.

In particular embodiments, x=y=19 and in (N)x each modified ribonucleotide is modified so as to have a 2′-O-methyl on its sugar and the ribonucleotide located at the middle of (N)x is unmodified. Accordingly, in a compound wherein x=19, (N)x comprises 2′-O-methyl sugar modified ribonucleotides at positions 1, 3, 5, 7, 9, 11, 13, 15, 17 and 19. In other embodiments, (N)x comprises 2′O Me modified ribonucleotides at positions 2, 4, 6, 8, 11, 13, 15, 17 and 19 and may further comprise at least one abasic or inverted abasic unconventional moiety for example in position 5. In other embodiments, (N)x comprises 2′O Me modified ribonucleotides at positions 2, 4, 8, 11, 13, 15, 17 and 19 and may further comprise at least one abasic or inverted abasic unconventional moiety for example in position 6. In other embodiments, (N)x comprises 2′O Me modified ribonucleotides at positions 2, 4, 6, 8, 11, 13, 17 and 19 and may further comprise at least one abasic or inverted abasic unconventional moiety for example in position 15. In other embodiments, (N)x comprises 2′O Me modified ribonucleotides at positions 2, 4, 6, 8, 11, 13, 15, 17 and 19 and may further comprise at least one abasic or inverted abasic unconventional moiety for example in position 14. In other embodiments, (N)x comprises 2′O Me modified ribonucleotides at positions 1, 2, 3, 7, 9, 11, 13, 15, 17 and 19 and may further comprise at least one abasic or inverted abasic unconventional moiety for example in position 5. In other embodiments, (N)x comprises 2′O Me modified ribonucleotides at positions 1, 2, 3, 5, 7, 9, 11, 13, 15, 17 and 19 and may further comprise at least one abasic or inverted abasic unconventional moiety for example in position 6. In other embodiments, (N)x comprises 2′O Me modified ribonucleotides at positions 1, 2, 3, 5, 7, 9, 11, 13, 17 and 19 and may further comprise at least one abasic or inverted abasic unconventional moiety for example in position 15. In other embodiments, (N)x comprises 2′O Me modified ribonucleotides at positions 1, 2, 3, 5, 7, 9, 11, 13, 15, 17 and 19 and may further comprise at least one abasic or inverted abasic unconventional moiety for example in position 14. In other embodiments, (N)x comprises 2′O Me modified ribonucleotides at positions 2, 4, 6, 7, 9, 11, 13, 15, 17 and 19 and may further comprise at least one abasic or inverted abasic unconventional moiety for example in position 5. In other embodiments, (N)x comprises 2′O Me modified ribonucleotides at positions 1, 2, 4, 6, 7, 9, 11, 13, 15, 17 and 19 and may further comprise at least one abasic or inverted abasic unconventional moiety for example in position 5. In other embodiments, (N)x comprises 2′O Me modified ribonucleotides at positions 2, 4, 6, 8, 11, 13, 14, 16, 17 and 19 and may further comprise at least one abasic or inverted abasic unconventional moiety for example in position 15. In other embodiments, (N)x comprises 2′O Me modified ribonucleotides at positions 1, 2, 3, 5, 7, 9, 11, 13, 14, 16, 17 and 19 and may further comprise at least one abasic or inverted abasic unconventional moiety for example in position 15. In other embodiments, (N)x comprises 2′O Me modified ribonucleotides at positions 2, 4, 6, 8, 11, 13, 15, 17 and 19 and may further comprise at least one abasic or inverted abasic unconventional moiety for example in position 7. In other embodiments, (N)x comprises 2′O Me modified ribonucleotides at positions 2, 4, 6, 11, 13, 15, 17 and 19 and may further comprise at least one abasic or inverted abasic unconventional moiety for example in position 8. In other embodiments, (N)x comprises 2′O Me modified ribonucleotides at positions 2, 4, 6, 8, 11, 13, 15, 17 and 19 and may further comprise at least one abasic or inverted abasic unconventional moiety for example in position 9. In other embodiments, (N)x comprises 2′O Me modified ribonucleotides at positions 2, 4, 6, 8, 11, 13, 15, 17 and 19 and may further comprise at least one abasic or inverted abasic unconventional moiety for example in position 10. In other embodiments, (N)x comprises 2′O Me modified ribonucleotides at positions 2, 4, 6, 8, 13, 15, 17 and 19 and may further comprise at least one abasic or inverted abasic unconventional moiety for example in position 11. In other embodiments, (N)x comprises 2′O Me modified ribonucleotides at positions 2, 4, 6, 8, 11, 13, 15, 17 and 19 and may further comprise at least one abasic or inverted abasic unconventional moiety for example in position 12. In other embodiments, (N)x comprises 2′O Me modified ribonucleotides at positions 2, 4, 6, 8, 11, 15, 17 and 19 and may further comprise at least one abasic or inverted abasic unconventional moiety for example in position 13.

In yet other embodiments (N)x comprises at least one nucleotide mismatch relative to the one of the SOX9, ASPP1, CTSD, CAPNS1, FAS and FAS ligand genes. In certain preferred embodiments, (N)x comprises a single nucleotide mismatch on position 5, 6, or 14. In one embodiment of Structure (C), at least two nucleotides at either or both the 5′ and 3′ termini of (N′)y are joined by a 2′-5′ phosphodiester bond. In certain preferred embodiments x=y=19 or x=y=23; in (N)x the nucleotides alternate between modified ribonucleotides and unmodified ribonucleotides, each modified ribonucleotide being modified so as to have a 2′-O-methyl on its sugar and the ribonucleotide located at the middle of (N)x being unmodified; and three nucleotides at the 3′ terminus of (N′)y are joined by two 2′-5′ phosphodiester bonds (set forth herein as Structure I). In other preferred embodiments, x=y=19 or x=y=23; in (N)x the nucleotides alternate between modified ribonucleotides and unmodified ribonucleotides, each modified ribonucleotide being modified so as to have a 2′-O-methyl on its sugar and the ribonucleotide located at the middle of (N)x being unmodified; and four consecutive nucleotides at the 5′ terminus of (N′)y are joined by three 2′-5′ phosphodiester bonds. In a further embodiment, an additional nucleotide located in the middle position of (N)y may be modified with 2′-O-methyl on its sugar. In another preferred embodiment, in (N)x the nucleotides alternate between 2′-O-methyl modified ribonucleotides and unmodified ribonucleotides, and in (N′)y four consecutive nucleotides at the 5′ terminus are joined by three 2′-5′ phosphodiester bonds and the 5′ terminal nucleotide or two or three consecutive nucleotides at the 5′ terminus comprise 3′-O-methyl modifications.

In certain preferred embodiments of Structure C, x=y=19 and in (N′)y, at least one position comprises an abasic or inverted abasic unconventional moiety, preferably five positions comprises an abasic or inverted abasic unconventional moieties. In various embodiments, the following positions comprise an abasic or inverted abasic: positions 1 and 16-19, positions 15-19, positions 1-2 and 17-19, positions 1-3 and 18-19, positions 1-4 and 19 and positions 1-5. (N′)y may further comprise at least one LNA nucleotide.

In certain preferred embodiments of Structure C, x=y=19 and in (N′)y the nucleotide in at least one position comprises a mirror nucleotide, a deoxyribonucleotide and a nucleotide joined to an adjacent nucleotide by a 2′-5′ internucleotide bond.

In certain preferred embodiments of Structure C, x=y=19 and (N′)y comprises a mirror nucleotide. In various embodiments the mirror nucleotide is an L-DNA nucleotide. In certain embodiments the L-DNA is L-deoxyribocytidine. In some embodiments (N′)y comprises L-DNA at position 18. In other embodiments (N′)y comprises L-DNA at positions 17 and 18. In certain embodiments (N′)y comprises L-DNA substitutions at positions 2 and at one or both of positions 17 and 18. In certain embodiments (N′)y further comprises a 5′ terminal cap nucleotide such as 5′-O-methyl DNA or an abasic or inverted abasic moiety as an overhang.

In yet other embodiments (N′)y comprises a DNA at position 15 and L-DNA at one or both of positions 17 and 18. In that structure, position 2 may further comprise an L-DNA or an abasic unconventional moiety.

Other embodiments of Structure C are envisaged wherein x=y=21 or wherein x=y=23; in these embodiments the modifications for (N′)y discussed above instead of being on positions 15, 16, 17, 18 are on positions 17, 18, 19, 20 for 21 mer and on positions 19, 20, 21, 22 for 23 mer; similarly the modifications at one or both of positions 17 and 18 are on one or both of positions 19 or 20 for the 21 mer and one or both of positions 21 and 22 for the 23 mer. All modifications in the 19 mer are similarly adjusted for the 21 and 23 mers.

According to various embodiments of Structure (C), in (N′)y 2, 3, 4, 5, 6, 7, 8, 9, 10, 11, 12, 13 or 14 consecutive ribonucleotides at the 3′ terminus are linked by 2′-5′ internucleotide linkages In one preferred embodiment, four consecutive nucleotides at the 3′ terminus of (N′)y are joined by three 2′-5′ phosphodiester bonds, wherein one or more of the 2′-5′ nucleotides which form the 2′-5′ phosphodiester bonds further comprises a 3′-O-methyl sugar modification. Preferably the 3′ terminal nucleotide of (N′)y comprises a 2′-O-methyl sugar modification. In certain preferred embodiments of Structure C, x=y=19 and in (N′)y two or more consecutive nucleotides at positions 15, 16, 17, 18 and 19 comprise a nucleotide joined to an adjacent nucleotide by a 2′-5′ internucleotide bond. In various embodiments the nucleotide forming the 2′-5′ internucleotide bond comprises a 3′ deoxyribose nucleotide or a 3′ methoxy nucleotide. In some embodiments the nucleotides at positions 17 and 18 in (N′)y are joined by a 2′-5′ internucleotide bond. In other embodiments the nucleotides at positions 16, 17, 18, 16-17, 17-18, or 16-18 in (N′)y are joined by a 2′-5′ internucleotide bond.

In certain embodiments (N′)y comprises an L-DNA at position 2 and 2′-5′ internucleotide bonds at positions 16-17, 17-18, or 16-18. In certain embodiments (N′)y comprises 2′-5′ internucleotide bonds at positions 16-17, 17-18, or 16-18 and a 5′ terminal cap nucleotide.

According to various embodiments of Structure (C), in (N′)y 2, 3, 4, 5, 6, 7, 8, 9, 10, 11, 12, 13 or 14 consecutive nucleotides at either terminus or 2-8 modified nucleotides at each of the 5′ and 3′ termini are independently mirror nucleotides. In some embodiments the mirror nucleotide is an L-ribonucleotide. In other embodiments the mirror nucleotide is an L-deoxyribonucleotide. The mirror nucleotide may further be modified at the sugar or base moiety or in an internucleotide linkage.

In one preferred embodiment of Structure (C), the 3′ terminal nucleotide or two or three consecutive nucleotides at the 3′ terminus of (N′)y are L-deoxyribonucleotides.

In other embodiments of Structure (C), in (N′)y 2, 3, 4, 5, 6, 7, 8, 9, 10, 11, 12, 13 or 14 consecutive ribonucleotides at either terminus or 2-8 modified nucleotides at each of the 5′ and 3′ termini are independently 2′ sugar modified nucleotides. In some embodiments the 2′ sugar modification comprises the presence of an amino, a fluoro, an alkoxy or an alkyl moiety. In certain embodiments the 2′ sugar modification comprises a methoxy moiety (2′-OMe). In one series of preferred embodiments, three, four or five consecutive nucleotides at the 5′ terminus of (N′)y comprise the 2′-OMe modification. In another preferred embodiment, three consecutive nucleotides at the 3′ terminus of (N′)y comprise the 2′-O-methyl modification.

In some embodiments of Structure (C), in (N′)y 2, 3, 4, 5, 6, 7, 8, 9, 10, 11, 12, 13 or 14 consecutive ribonucleotides at either or 2-8 modified nucleotides at each of the 5′ and 3′ termini are independently bicyclic nucleotide. In various embodiments the bicyclic nucleotide is a locked nucleic acid (LNA). A 2′-O, 4′-C-ethylene-bridged nucleic acid (ENA) is a species of LNA (see below).

In various embodiments (N′)y comprises modified nucleotides at the 5′ terminus or at both the 3′ and 5′ termini.

In some embodiments of Structure (C), at least two nucleotides at either or both the 5′ and 3′ termini of (N′)y are joined by P-ethoxy backbone modifications. In certain preferred embodiments x=y=19 or x=y=23; in (N)x the nucleotides alternate between modified ribonucleotides and unmodified ribonucleotides, each modified ribonucleotide being modified so as to have a 2′-O-methyl on its sugar and the ribonucleotide located at the middle position of (N)x being unmodified; and four consecutive nucleotides at the 3′ terminus or at the 5′ terminus of (N′)y are joined by three P-ethoxy backbone modifications. In another preferred embodiment, three consecutive nucleotides at the 3′ terminus or at the 5′ terminus of (N′)y are joined by two P-ethoxy backbone modifications.

In some embodiments of Structure (C), in (N′)y 2, 3, 4, 5, 6, 7 or 8, consecutive ribonucleotides at each of the 5′ and 3′ termini are independently mirror nucleotides, nucleotides joined by 2′-5′ phosphodiester bond, 2′ sugar modified nucleotides or bicyclic nucleotide. In one embodiment, the modification at the 5′ and 3′ termini of (N′)y is identical. In one preferred embodiment, four consecutive nucleotides at the 5′ terminus of (N′)y are joined by three 2′-5′ phosphodiester bonds and three consecutive nucleotides at the 3′ terminus of (N′)y are joined by two 2′-5′ phosphodiester bonds. In another embodiment, the modification at the 5′ terminus of (N′)y is different from the modification at the 3′ terminus of (N′)y. In one specific embodiment, the modified nucleotides at the 5′ terminus of (N′)y are mirror nucleotides and the modified nucleotides at the 3′ terminus of (N′)y are joined by 2′-5′ phosphodiester bond. In another specific embodiment, three consecutive nucleotides at the 5′ terminus of (N′)y are LNA nucleotides and three consecutive nucleotides at the 3′ terminus of (N′)y are joined by two 2′-5′ phosphodiester bonds. In (N)x the nucleotides alternate between modified ribonucleotides and unmodified ribonucleotides, each modified ribonucleotide being modified so as to have a 2′-O-methyl on its sugar and the ribonucleotide located at the middle of (N)x being unmodified, or the ribonucleotides in (N)x being unmodified

In another embodiment of Structure (C), the present invention provides a compound wherein x=y=19 or x=y=23; in (N)x the nucleotides alternate between modified ribonucleotides and unmodified ribonucleotides, each modified ribonucleotide being modified so as to have a 2′-O-methyl on its sugar and the ribonucleotide located at the middle of (N)x being unmodified; three nucleotides at the 3′ terminus of (N′)y are joined by two 2′-5′ phosphodiester bonds and three nucleotides at the 5′ terminus of (N′)y are LNA such as ENA.

In another embodiment of Structure (C), five consecutive nucleotides at the 5′ terminus of (N′)y comprise the 2′-O-methyl sugar modification and two consecutive nucleotides at the 3′ terminus of (N′)y are L-DNA.

In yet another embodiment, the present invention provides a compound wherein x=y=19 or x=y=23; (N)x consists of unmodified ribonucleotides; three consecutive nucleotides at the 3′ terminus of (N′)y are joined by two 2′-5′ phosphodiester bonds and three consecutive nucleotides at the 5′ terminus of (N′)y are LNA such as ENA.

According to other embodiments of Structure (C), in (N′)y the 5′ or 3′ terminal nucleotide, or 2, 3, 4, 5 or 6 consecutive nucleotides at either termini or 1-4 modified nucleotides at each of the 5′ and 3′ termini are independently phosphonocarboxylate or phosphinocarboxylate nucleotides (PACE nucleotides). In some embodiments the PACE nucleotides are deoxyribonucleotides. In some preferred embodiments in (N′)y, 1 or 2 consecutive nucleotides at each of the 5′ and 3′ termini are PACE nucleotides.

In additional embodiments, the present invention provides a compound having Structure (D):

5′(N)x-Z3′ antisense strand (D)

3′Z′—(N′)y5′ sense strand

wherein each of N and N′ is a nucleotide selected from an unmodified ribonucleotide, a modified ribonucleotide, an unmodified deoxyribonucleotide or a modified deoxyribonucleotide;
wherein each of (N)x and (N′)y is an oligomer in which each consecutive nucleotide is joined to the next nucleotide by a covalent bond and each of x and y is an integer between 18 and 40;
wherein (N)x comprises unmodified ribonucleotides further comprising one modified nucleotide at the 3′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein (N′)y comprises unmodified ribonucleotides further comprising one modified nucleotide at the 5′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein in each of (N)x and (N′)y modified and unmodified nucleotides are not alternating;
wherein each of Z and Z′ may be present or absent, but if present is 1-5 deoxyribonucleotides covalently attached at the 3′ terminus of any oligomer to which it is attached;
wherein the sequence of (N′)_{y} is a sequence substantially complementary to (N)x; and
wherein (N)ₓ comprises an antisense sequence substantially identical to an antisense sequence set forth in any one of Tables A-AQ.

In one embodiment of Structure (D), x=y=19 or x=y=23; (N)x comprises unmodified ribonucleotides in which two consecutive nucleotides linked by one 2′-5′ internucleotide linkage at the 3′ terminus; and (N′)y comprises unmodified ribonucleotides in which two consecutive nucleotides linked by one 2′-5′ internucleotide linkage at the 5′ terminus.

In some embodiments, x=y=19 or x=y=23; (N)x comprises unmodified ribonucleotides in which three consecutive nucleotides at the 3′ terminus are joined together by two 2′-5′ phosphodiester bonds; and (N′)y comprises unmodified ribonucleotides in which four consecutive nucleotides at the 5′ terminus are joined together by three 2′-5′ phosphodiester bonds (set forth herein as Structure II).

According to various embodiments of Structure (D) 2, 3, 4, 5, 6, 7, 8, 9, 10, 11, 12, 13 or 14 consecutive ribonucleotides starting at the ultimate or penultimate position of the 3′ terminus of (N)x and 2, 3, 4, 5, 6, 7, 8, 9, 10, 11, 12, 13 or 14 consecutive ribonucleotides starting at the ultimate or penultimate position of the 5′ terminus of (N′)y are linked by 2′-5′ internucleotide linkages.

According to one preferred embodiment of Structure (D), four consecutive nucleotides at the 5′ terminus of (N′)y are joined by three 2′-5′ phosphodiester bonds and three consecutive nucleotides at the 3′ terminus of (N′)x are joined by two 2′-5′ phosphodiester bonds. Three nucleotides at the 5′ terminus of (N′)y and two nucleotides at the 3′ terminus of (N′)x may also comprise 3′-O-methyl modifications.

According to various embodiments of Structure (D), 2, 3, 4, 5, 6, 7, 8, 9, 10, 11, 12, 13 or 14 consecutive nucleotides starting at the ultimate or penultimate position of the 3′ terminus of (N)x and 2, 3, 4, 5, 6, 7, 8, 9, 10, 11, 12, 13 or 14 consecutive ribonucleotides starting at the ultimate or penultimate position of the 5′ terminus of (N′)y are independently mirror nucleotides. In some embodiments the mirror is an L-ribonucleotide. In other embodiments the mirror nucleotide is L-deoxyribonucleotide.

In other embodiments of Structure (D), 2, 3, 4, 5, 6, 7, 8, 9, 10, 11, 12, 13 or 14 consecutive ribonucleotides starting at the ultimate or penultimate position of the 3′ terminus of (N)x and 2, 3, 4, 5, 6, 7, 8, 9, 10, 11, 12, 13 or 14 consecutive ribonucleotides starting at the ultimate or penultimate position of the 5′ terminus of (N′)y are independently 2′ sugar modified nucleotides. In some embodiments the 2′ sugar modification comprises the presence of an amino, a fluoro, an alkoxy or an alkyl moiety. In certain embodiments the 2′ sugar modification comprises a methoxy moiety (2′-OMe).

In one preferred embodiment of Structure (D), five consecutive nucleotides at the 5′ terminus of (N′)y comprise the 2′-O-methyl modification and five consecutive nucleotides at the 3′ terminus of (N′)x comprise the 2′-O-methyl modification. In another preferred embodiment of Structure (D), ten consecutive nucleotides at the 5′ terminus of (N′)y comprise the 2′-O-methyl modification and five consecutive nucleotides at the 3′ terminus of (N′)x comprise the 2′-O-methyl modification. In another preferred embodiment of Structure (D), thirteen consecutive nucleotides at the 5′ terminus of (N′)y comprise the 2′-O-methyl modification and five consecutive nucleotides at the 3′ terminus of (N′)x comprise the 2′-O-methyl modification.

In some embodiments of Structure (D), in (N′)y 2, 3, 4, 5, 6, 7, 8, 9, 10, 11, 12, 13 or 14 consecutive ribonucleotides starting at the ultimate or penultimate position of the 3′ terminus of (N)x and 2, 3, 4, 5, 6, 7, 8, 9, 10, 11, 12, 13 or 14 consecutive ribonucleotides starting at the ultimate or penultimate position of the 5′ terminus of (N′)y are independently a bicyclic nucleotide. In various embodiments the bicyclic nucleotide is a locked nucleic acid (LNA) such as a 2′-O, 4′-C-ethylene-bridged nucleic acid (ENA).

In various embodiments of Structure (D), (N′)y comprises a modified nucleotide selected from a bicyclic nucleotide, a 2′ sugar modified nucleotide, a mirror nucleotide, an altritol nucleotide or a nucleotide joined to an adjacent nucleotide by an internucleotide linkage selected from a 2′-5′ phosphodiester bond, a P-alkoxy linkage or a PACE linkage;

In various embodiments of Structure (D), (N)x comprises a modified nucleotide selected from a bicyclic nucleotide, a 2′ sugar modified nucleotide, a mirror nucleotide, an altritol nucleotide or a nucleotide joined to an adjacent nucleotide by an internucleotide linkage selected from a 2′-5′ phosphodiester bond, a P-alkoxy linkage or a PACE linkage;

In embodiments wherein each of the 3′ and 5′ termini of the same strand comprises a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond.

In one specific embodiment of Structure (D), five consecutive nucleotides at the 5′ terminus of (N′)y comprise the 2′-O-methyl modification and two consecutive nucleotides at the 3′ terminus of (N′)y are L-DNA. In addition, the compound may further comprise five consecutive 2′-O-methyl modified nucleotides at the 3′ terminus of (N′)x.

In various embodiments of Structure (D), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are mirror nucleotides.

In additional embodiments, the present invention provides a compound having Structure (E):

5′(N)x-Z3′ antisense strand (E)

3′Z′—(N′)y5′ sense strand

wherein each of N and N′ is a nucleotide selected from an unmodified ribonucleotide, a modified ribonucleotide, an unmodified deoxyribonucleotide or a modified deoxyribonucleotide;
wherein each of (N)x and (N′)y is an oligomer in which each consecutive nucleotide is joined to the next nucleotide by a covalent bond and each of x and y is an integer between 18 and 40;
wherein (N)x comprises unmodified ribonucleotides further comprising one modified nucleotide at the 5′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein (N′)y comprises unmodified ribonucleotides further comprising one modified nucleotide at the 3′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein in each of (N)x and (N′)y modified and unmodified nucleotides are not alternating;
wherein each of Z and Z′ may be present or absent, but if present is 1-5 deoxyribonucleotides covalently attached at the 3′ terminus of any oligomer to which it is attached;
wherein the sequence of (N′)_{y} is a sequence substantially complementary to (N)x; and
wherein the sequence of (N)ₓ comprises an antisense sequence substantially identical to an antisense sequence set forth in any one of Tables A-AQ.

In certain preferred embodiments the ultimate nucleotide at the 5′ terminus of (N)x is unmodified.

According to various embodiments of Structure (E) 2, 3, 4, 5, 6, 7, 8, 9, 10, 11, 12, 13 or 14 consecutive ribonucleotides starting at the ultimate or penultimate position of the 5′ terminus of (N)x, preferably starting at the 5′ penultimate position, and 2, 3, 4, 5, 6, 7, 8, 9, 10, 11, 12, 13 or 14 consecutive ribonucleotides starting at the ultimate or penultimate position of the 3′ terminus of (N′)y are linked by 2′-5′ internucleotide linkages.

According to various embodiments of Structure (E), 2, 3, 4, 5, 6, 7, 8, 9, 10, 11, 12, 13 or 14 consecutive nucleotides starting at the ultimate or penultimate position of the 5′ terminus of (N)x, preferably starting at the 5′ penultimate position, and 2, 3, 4, 5, 6, 7, 8, 9, 10, 11, 12, 13 or 14 consecutive nucleotides starting at the ultimate or penultimate position of the 3′ terminus of (N′)y are independently mirror nucleotides. In some embodiments the mirror is an L-ribonucleotide. In other embodiments the mirror nucleotide is L-deoxyribonucleotide.

In other embodiments of Structure (E), 2, 3, 4, 5, 6, 7, 8, 9, 10, 11, 12, 13 or 14 consecutive ribonucleotides starting at the ultimate or penultimate position of the 5′ terminus of (N)x, preferably starting at the 5′ penultimate position, and 2, 3, 4, 5, 6, 7, 8, 9, 10, 11, 12, 13 or 14 consecutive ribonucleotides starting at the ultimate or penultimate position of the 3′ terminus of (N′)y are independently 2′ sugar modified nucleotides. In some embodiments the 2′ sugar modification comprises the presence of an amino, a fluoro, an alkoxy or an alkyl moiety. In certain embodiments the 2′ sugar modification comprises a methoxy moiety (2′-OMe).

In some embodiments of Structure (E), in (N′)y 2, 3, 4, 5, 6, 7, 8, 9, 10, 11, 12, 13 or 14 consecutive ribonucleotides starting at the ultimate or penultimate position of the 5′ terminus of (N)x, preferably starting at the 5′ penultimate position, and 2, 3, 4, 5, 6, 7, 8, 9, 10, 11, 12, 13 or 14 consecutive ribonucleotides starting at the ultimate or penultimate position of the 3′ terminus of (N′)y are independently a bicyclic nucleotide. In various embodiments the bicyclic nucleotide is a locked nucleic acid (LNA) such as a 2′-O, 4′-C-ethylene-bridged nucleic acid (ENA).

In various embodiments of Structure (E), (N′)y comprises modified nucleotides selected from a bicyclic nucleotide, a 2′ sugar modified nucleotide, a mirror nucleotide, an altritol nucleotide, a nucleotide joined to an adjacent nucleotide by a P-alkoxy backbone modification or a nucleotide joined to an adjacent nucleotide by an internucleotide linkage selected from a 2′-5′ phosphodiester bond, a P-alkoxy linkage or a PACE linkage at the 3′ terminus or at each of the 3′ and 5′ termini.

In various embodiments of Structure (E), (N)x comprises a modified nucleotide selected from a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage at the 5′ terminus or at each of the 3′ and 5′ termini.

In one embodiment where both 3′ and 5′ termini of the same strand comprise a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond.

In various embodiments of Structure (E), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are mirror nucleotides.

In additional embodiments, the present invention provides a compound having Structure (F):

5′(N)x-Z3′ antisense strand (F)

3′Z′—(N′)y5′ sense strand

wherein each of N and N′ is a nucleotide selected from an unmodified ribonucleotide, a modified ribonucleotide, an unmodified deoxyribonucleotide or a modified deoxyribonucleotide;
wherein each of (N)x and (N′)y is an oligomer in which each consecutive nucleotide is joined to the next nucleotide by a covalent bond and each of x and y is an integer between 18 and 40;
wherein each of (N)x and (N′)y comprise unmodified ribonucleotides in which each of (N)x and (N′)y independently comprise one modified nucleotide at the 3′ terminal or penultimate position wherein the modified nucleotide is selected from the group consisting of a bicyclic nucleotide, a 2′ sugar modified nucleotide, a mirror nucleotide, a nucleotide joined to an adjacent nucleotide by a P-alkoxy backbone modification or a nucleotide joined to an adjacent nucleotide by a 2′-5′ phosphodiester bond;
wherein in each of (N)x and (N′)y modified and unmodified nucleotides are not alternating;
wherein each of Z and Z′ may be present or absent, but if present is 1-5 deoxyribonucleotides covalently attached at the 3′ terminus of any oligomer to which it is attached;
wherein the sequence of (N′)_{y} is a sequence substantially complementary to (N)x; and
wherein the sequence of (N)ₓ comprises an antisense sequence substantially identical to an antisense sequence set forth in any one of Tables A-AQ.

In some embodiments of Structure (F), x=y=19 or x=y=23; (N′)y comprises unmodified ribonucleotides in which two consecutive nucleotides at the 3′ terminus comprises two consecutive mirror deoxyribonucleotides; and (N)x comprises unmodified ribonucleotides in which one nucleotide at the 3′ terminus comprises a mirror deoxyribonucleotide (set forth as Structure III).

According to various embodiments of Structure (F) 2, 3, 4, 5, 6, 7, 8, 9, 10, 11, 12, 13 or 14 consecutive ribonucleotides independently beginning at the ultimate or penultimate position of the 3′ termini of (N)x and (N′)y are linked by 2′-5′ internucleotide linkages.

According to one preferred embodiment of Structure (F), three consecutive nucleotides at the 3′ terminus of (N′)y are joined by two 2′-5′ phosphodiester bonds and three consecutive nucleotides at the 3′ terminus of (N′)x are joined by two 2′-5′ phosphodiester bonds.

According to various embodiments of Structure (F), 2, 3, 4, 5, 6, 7, 8, 9, 10, 11, 12, 13 or 14 consecutive nucleotides independently beginning at the ultimate or penultimate position of the 3′ termini of (N)x and (N′)y are independently mirror nucleotides. In some embodiments the mirror nucleotide is an L-ribonucleotide. In other embodiments the mirror nucleotide is an L-deoxyribonucleotide.

In other embodiments of Structure (F), 2, 3, 4, 5, 6, 7, 8, 9, 10, 11, 12, 13 or 14 consecutive ribonucleotides independently beginning at the ultimate or penultimate position of the 3′ termini of (N)x and (N′)y are independently 2′ sugar modified nucleotides. In some embodiments the 2′ sugar modification comprises the presence of an amino, a fluoro, an alkoxy or an alkyl moiety. In certain embodiments the 2′ sugar modification comprises a methoxy moiety (2′-OMe).

In some embodiments of Structure (F), 2, 3, 4, 5, 6, 7, 8, 9, 10, 11, 12, 13 or 14 consecutive ribonucleotides independently beginning at the ultimate or penultimate position of the 3′ termini of (N)x and (N′)y are independently a bicyclic nucleotide. In various embodiments the bicyclic nucleotide is a locked nucleic acid (LNA) such as a 2′-O, 4′-C-ethylene-bridged nucleic acid (ENA).

In various embodiments of Structure (F), (N′)y comprises a modified nucleotide selected from a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage at the 3′ terminus or at both the 3′ and 5′ termini.

In various embodiments of Structure (F), (N)x comprises a modified nucleotide selected from a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage at the 3′ terminus or at each of the 3′ and 5′ termini.

In one embodiment where each of 3′ and 5′ termini of the same strand comprise a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond.

In various embodiments of Structure (F), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are mirror nucleotides.

In additional embodiments, the present invention provides a compound having Structure (G):

5′(N)x-Z3′ antisense strand (G)

3′Z′—(N′)y5′ sense strand

wherein each of N and N′ is a nucleotide selected from an unmodified ribonucleotide, a modified ribonucleotide, an unmodified deoxyribonucleotide or a modified deoxyribonucleotide;
wherein each of (N)x and (N′)y is an oligomer in which each consecutive nucleotide is joined to the next nucleotide by a covalent bond and each of x and y is an integer between 18 and 40;
wherein each of (N)x and (N′)y comprise unmodified ribonucleotides in which each of (N)x and (N′)y independently comprise one modified nucleotide at the 5′ terminal or penultimate position wherein the modified nucleotide is selected from the group consisting of a bicyclic nucleotide, a 2′ sugar modified nucleotide, a mirror nucleotide, a nucleotide joined to an adjacent nucleotide by a P-alkoxy backbone modification or a nucleotide joined to an adjacent nucleotide by a 2′-5′ phosphodiester bond;
wherein for (N)x the modified nucleotide is preferably at penultimate position of the 5′ terminal;
wherein in each of (N)x and (N′)y modified and unmodified nucleotides are not alternating;
wherein each of Z and Z′ may be present or absent, but if present is 1-5 deoxyribonucleotides covalently attached at the 3′ terminus of any oligomer to which it is attached;
wherein the sequence of (N′)_{y} is a sequence substantially complementary to (N)x; and
wherein the sequence of (N)ₓ comprises an antisense sequence substantially identical to an antisense sequence set forth in any one of Tables A-AQ.

In some embodiments of Structure (G), x=y=19 or x=y=23.

According to various embodiments of Structure (G) 2, 3, 4, 5, 6, 7, 8, 9, 10, 11, 12, 13 or 14 consecutive ribonucleotides independently beginning at the ultimate or penultimate position of the 5′ termini of (N)x and (N′)y are linked by 2′-5′ internucleotide linkages. For (N)x the modified nucleotides preferably starting at the penultimate position of the 5′ terminal.

According to various embodiments of Structure (G), 2, 3, 4, 5, 6, 7, 8, 9, 10, 11, 12, 13 or 14 consecutive nucleotides independently beginning at the ultimate or penultimate position of the 5′ termini of (N)x and (N′)y are independently mirror nucleotides. In some embodiments the mirror nucleotide is an L-ribonucleotide. In other embodiments the mirror nucleotide is an L-deoxyribonucleotide. For (N)x the modified nucleotides preferably starting at the penultimate position of the 5′ terminal.

In other embodiments of Structure (G), 2, 3, 4, 5, 6, 7, 8, 9, 10, 11, 12, 13 or 14 consecutive ribonucleotides independently beginning at the ultimate or penultimate position of the 5′ termini of (N)x and (N′)y are independently 2′ sugar modified nucleotides. In some embodiments the 2′ sugar modification comprises the presence of an amino, a fluoro, an alkoxy or an alkyl moiety. In certain embodiments the 2′ sugar modification comprises a methoxy moiety (2′-OMe). In some preferred embodiments the consecutive modified nucleotides preferably begin at the penultimate position of the 5′ terminus of (N)x.

In one preferred embodiment of Structure (G), five consecutive ribonucleotides at the 5′ terminus of (N′)y comprise a 2′-O-methyl modification and one ribonucleotide at the 5′ penultimate position of (N′)x comprises a 2′-O-methyl modification. In another preferred embodiment of Structure (G), five consecutive ribonucleotides at the 5′ terminus of (N′)y comprise a 2′-O-methyl modification and two consecutive ribonucleotides at the 5′ terminal position of (N′)x comprise a 2′-O-methyl modification.

In some embodiments of Structure (G), 2, 3, 4, 5, 6, 7, 8, 9, 10, 11, 12, 13 or 14 consecutive ribonucleotides independently beginning at the ultimate or penultimate position of the 5′ termini of (N)x and (N′)y are bicyclic nucleotides. In various embodiments the bicyclic nucleotide is a locked nucleic acid (LNA) such as a 2′-O, 4′-C-ethylene-bridged nucleic acid (ENA). In some preferred embodiments the consecutive modified nucleotides preferably begin at the penultimate position of the 5′ terminus of (N)x.

In various embodiments of Structure (G), (N′)y comprises a modified nucleotide selected from a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage at the 5′ terminus or at each of the 3′ and 5′ termini.

In various embodiments of Structure (G), (N)x comprises a modified nucleotide selected from a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage at the 5′ terminus or at each of the 3′ and 5′ termini.

In one embodiment where each of 3′ and 5′ termini of the same strand comprise a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond. In various embodiments of Structure (G), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are mirror nucleotides.

In additional embodiments, the present invention provides a compound having Structure (H):

5′(N)x-Z3′ antisense strand (H)

3′Z′—(N′)y5′ sense strand

wherein each of N and N′ is a nucleotide selected from an unmodified ribonucleotide, a modified ribonucleotide, an unmodified deoxyribonucleotide or a modified deoxyribonucleotide;
wherein each of (N)x and (N′)y is an oligomer in which each consecutive nucleotide is joined to the next nucleotide by a covalent bond and each of x and y is an integer between 18 and 40;
wherein (N)x comprises unmodified ribonucleotides further comprising one modified nucleotide at the 3′ terminal or penultimate position or the 5′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein (N′)y comprises unmodified ribonucleotides further comprising one modified nucleotide at an internal position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein in each of (N)x and (N′)y modified and unmodified nucleotides are not alternating;
wherein each of Z and Z′ may be present or absent, but if present is 1-5 deoxyribonucleotides covalently attached at the 3′ terminus of any oligomer to which it is attached;
wherein the sequence of (N′)_{y} is a sequence substantially complementary to (N)x; and
wherein the sequence of (N)ₓ comprises an antisense sequence substantially identical to an antisense sequence set forth in any one of Tables A-AQ.

In one embodiment of Structure (H), 2, 3, 4, 5, 6, 7, 8, 9, 10, 11, 12, 13 or 14 consecutive ribonucleotides independently beginning at the ultimate or penultimate position of the 3′ terminus or the 5′ terminus or both termini of (N)x are independently 2′ sugar modified nucleotides, bicyclic nucleotides, mirror nucleotides, altritol nucleotides or nucleotides joined to an adjacent nucleotide by a 2′-5′ phosphodiester bond and 2, 3, 4, 5, 6, 7, 8, 9, 10, 11, 12, 13 or 14 consecutive internal ribonucleotides in (N′)y are independently 2′ sugar modified nucleotides, bicyclic nucleotides, mirror nucleotides, altritol nucleotides or nucleotides joined to an adjacent nucleotide by a 2′-5′ phosphodiester bond. In some embodiments the 2′ sugar modification comprises the presence of an amino, a fluoro, an alkoxy or an alkyl moiety. In certain embodiments the 2′ sugar modification comprises a methoxy moiety (2′-OMe).

In another embodiment of Structure (H), 2, 3, 4, 5, 6, 7, 8, 9, 10, 11, 12, 13 or 14 consecutive ribonucleotides independently beginning at the ultimate or penultimate position of the 3′ terminus or the 5′ terminus or 2-8 consecutive nucleotides at each of 5′ and 3′ termini of (N′)y are independently 2′ sugar modified nucleotides, bicyclic nucleotides, mirror nucleotides, altritol nucleotides or nucleotides joined to an adjacent nucleotide by a 2′-5′ phosphodiester bond, and 2, 3, 4, 5, 6, 7, 8, 9, 10, 11, 12, 13 or 14 consecutive internal ribonucleotides in (N)x are independently 2′ sugar modified nucleotides, bicyclic nucleotides, mirror nucleotides, altritol nucleotides or nucleotides joined to an adjacent nucleotide by a 2′-5′ phosphodiester bond.

In one embodiment wherein each of 3′ and 5′ termini of the same strand comprises a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond.

In various embodiments of Structure (H), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are mirror nucleotides.

In one preferred embodiment of Structure (H), x=y=19; three consecutive ribonucleotides at the 9-11 nucleotide positions 9-11 of (N′)y comprise 2′-O-methyl modification and five consecutive ribonucleotides at the 3′ terminal position of (N′)x comprise 2′-O-methyl modification.

For all the above Structures (A)-(H), in various embodiments x=y and each of x and y is 19, 20, 21, 22 or 23. In certain embodiments, x=y=19. In yet other embodiments x=y=23. In additional embodiments the compound comprises modified ribonucleotides in alternating positions wherein each N at the 5′ and 3′ termini of (N)x are modified in their sugar residues and the middle ribonucleotide is not modified, e.g. ribonucleotide in position 10 in a 19-mer strand, position 11 in a 21 mer and position 12 in a 23-mer strand.

In some embodiments where x=y=21 or x=y=23 the position of modifications in the 19 mer are adjusted for the 21 and 23 mers with the proviso that the middle nucleotide of the antisense strand is preferably not modified.

In some embodiments, neither (N)x nor (N′)y are phosphorylated at the 3′ and 5′ termini. In other embodiments either or both (N)x and (N′)y are phosphorylated at the 3′ termini. In yet another embodiment, either or both (N)x and (N′)y are phosphorylated at the 3′ termini using non-cleavable phosphate groups. In yet another embodiment, either or both (N)x and (N′)y are phosphorylated at the terminal 2′ termini position using cleavable or non-cleavable phosphate groups. These particular siRNA compounds are also blunt ended and are non-phosphorylated at the termini; however, comparative experiments have shown that siRNA compounds phosphorylated at one or both of the 3′-termini have similar activity in vivo compared to the non-phosphorylated compounds.

In certain embodiments for all the above-mentioned Structures, the compound is blunt ended, for example wherein both Z and Z′ are absent. In an alternative embodiment, the compound comprises at least one 3′ overhang, wherein at least one of Z or Z′ is present. Z and Z′ independently comprises one or more covalently linked modified or non-modified nucleotides, for example inverted dT or dA; dT, LNA, mirror nucleotide and the like. In some embodiments each of Z and Z′ are independently selected from dT and dTdT. siRNA in which Z and/or Z′ is present have similar activity and stability as siRNA in which Z and Z′ are absent.

In certain embodiments for all the above-mentioned Structures, the compound comprises one or more phosphonocarboxylate and/or phosphinocarboxylate nucleotides (PACE nucleotides). In some embodiments the PACE nucleotides are deoxyribonucleotides and the phosphinocarboxylate nucleotides are phosphinoacetate nucleotides. Examples of PACE nucleotides and analogs are disclosed in U.S. Pat. Nos. 6,693,187 and 7,067,641, both incorporated herein by reference.

In certain embodiments for all the above-mentioned Structures, the compound comprises one or more locked nucleic acids (LNA) also defined as bridged nucleic acids or bicyclic nucleotides. Preferred locked nucleic acids are 2′-O, 4′-C-ethylene nucleosides (ENA) or 2′-O, 4′-C-methylene nucleosides. Other examples of LNA and ENA nucleotides are disclosed in WO 98/39352, WO 00/47599 and WO 99/14226, all incorporated herein by reference.

In certain embodiments for all the above-mentioned Structures, the compound comprises one or more altritol monomers (nucleotides), also defined as 1,5 anhydro-2-deoxy-D-altrito-hexitol (see for example, Allart, et al., 1998. Nucleosides & Nucleotides 17:1523-1526; Herdewijn et al., 1999. Nucleosides & Nucleotides 18:1371-1376; Fisher et al., 2007, NAR 35(4):1064-1074; all incorporated herein by reference).

The present invention explicitly excludes compounds in which each of N and/or N′ is a deoxyribonucleotide (D-A, D-C, D-G, D-T). In certain embodiments (N)x and (N′)y may comprise independently 1, 2, 3, 4, 5, 6, 7, 8, 9 or more deoxyribonucleotides. In certain embodiments the present invention provides a compound wherein each of N is an unmodified ribonucleotide and the 3′ terminal nucleotide or 2, 3, 4, 5, 6, 7, 8, 9, 10, 11, 12, 13 or 14 consecutive nucleotides at the 3′ terminus of (N′)y are deoxyribonucleotides. In yet other embodiments each of N is an unmodified ribonucleotide and the 5′ terminal nucleotide or 2, 3, 4, 5, 6, 7, 8, 9, 10, 11, 12, 13 or 14 consecutive nucleotides at the 5′ terminus of (N′)y are deoxyribonucleotides. In further embodiments the 5′ terminal nucleotide or 2, 3, 4, 5, 6, 7, 8, or 9 consecutive nucleotides at the 5′ terminus and 1, 2, 3, 4, 5, or 6 consecutive nucleotides at the 3′ termini of (N)x are deoxyribonucleotides and each of N′ is an unmodified ribonucleotide. In yet further embodiments (N)x comprises unmodified ribonucleotides and 1 or 2, 3 or 4 consecutive deoxyribonucleotides independently at each of the 5′ and 3′ termini and 1 or 2, 3, 4, 5 or 6 consecutive deoxyribonucleotides in internal positions; and each of N′ is an unmodified ribonucleotide. In certain embodiments the 3′ terminal nucleotide or 2, 3, 4, 5, 6, 7, 8, 9, 10, 11, 12 13 or 14 consecutive nucleotides at the 3′ terminus of (N′)y and the terminal 5′ nucleotide or 2, 3, 4, 5, 6, 7, 8, 9, 10, 11, 12 13 or 14 consecutive nucleotides at the 5′ terminus of (N)x are deoxyribonucleotides. The present invention excludes compounds in which each of N and/or N′ is a deoxyribonucleotide. In some embodiments the 5′ terminal nucleotide of N or 2 or 3 consecutive of N and 1, 2, or 3 of N′ is a deoxyribonucleotide. Certain examples of active DNA/RNA siRNA chimeras are disclosed in US patent publication 2005/0004064, and Ui-Tei, 2008 (NAR 36(7):2136-2151) incorporated herein by reference in their entirety.

Unless otherwise indicated, in preferred embodiments of the structures discussed herein the covalent bond between each consecutive N or N′ is a phosphodiester bond.

An additional novel molecule provided by the present invention is an oligonucleotide comprising consecutive nucleotides wherein a first segment of such nucleotides encode a first inhibitory RNA molecule, a second segment of such nucleotides encode a second inhibitory RNA molecule, and a third segment of such nucleotides encode a third inhibitory RNA molecule. Each of the first, the second and the third segment may comprise one strand of a double stranded RNA and the first, second and third segments may be joined together by a linker. Further, the oligonucleotide may comprise three double stranded segments joined together by one or more linker.

Thus, one molecule provided by the present invention is an oligonucleotide comprising consecutive nucleotides which encode three inhibitory RNA molecules; said oligonucleotide may possess a triple stranded structure, such that three double stranded arms are linked together by one or more linker, such as any of the linkers presented hereinabove. This molecule forms a “star”-like structure, and may also be referred to herein as RNAstar. Such structures are disclosed in PCT patent publication WO 2007/091269, assigned to the assignee of the present invention and incorporated herein in its entirety by reference.

A covalent bond refers to an internucleotide linkage linking one nucleotide monomer to an adjacent nucleotide monomer. A covalent bond includes for example, a phosphodiester bond, a phosphorothioate bond, a P-alkoxy bond, a P-carboxy bond and the like. The normal internucleoside linkage of RNA and DNA is a 3′ to 5′ phosphodiester linkage. In certain preferred embodiments a covalent bond is a phosphodiester bond. Covalent bond encompasses non-phosphorous-containing internucleoside linkages, such as those disclosed in WO 2004/041924 inter alia. Unless otherwise indicated, in preferred embodiments of the structures discussed herein the covalent bond between each consecutive N or N′ is a phosphodiester bond.

For all of the structures above, in some embodiments the oligonucleotide sequence of (N)x is fully complementary to the oligonucleotide sequence of (N′)y. In other embodiments (N)x and (N′)y are substantially complementary. In certain embodiments (N)x is fully complementary to a target sequence. In other embodiments (N)x is substantially complementary to a target sequence.

In some embodiments, neither (N)x nor (N′)y are phosphorylated at the 3′ and 5′ termini. In other embodiments either or both (N)x and (N′)y are phosphorylated at the 3′ termini (3′ Pi). In yet another embodiment, either or both (N)x and (N′)y are phosphorylated at the 3′ termini with non-cleavable phosphate groups. In yet another embodiment, either or both (N)x and (N′)y are phosphorylated at the terminal 2′ termini position using cleavable or non-cleavable phosphate groups. Further, the inhibitory nucleic acid molecules of the present invention may comprise one or more gaps and/or one or more nicks and/or one or more mismatches. Without wishing to be bound by theory, gaps, nicks and mismatches have the advantage of partially destabilizing the nucleic acid/siRNA, so that it may be more easily processed by endogenous cellular machinery such as DICER, DROSHA or RISC into its inhibitory components.

In the context of the present invention, a gap in a nucleic acid refers to the absence of one or more internal nucleotides in one strand, while a nick in a nucleic acid refers to the absence of an internucleotide linkage between two adjacent nucleotides in one strand. Any of the molecules of the present invention may contain one or more gaps and/or one or more nicks.

In one aspect the present invention provides a compound having Structure (I):

5′(N)x-Z3′ (antisense strand) (I)

3′Z′—(N′)y-z″5′ (sense strand)

wherein each of N and N′ is a ribonucleotide which may be unmodified or modified, or an unconventional moiety;
wherein each of (N)x and (N′)y is an oligonucleotide in which each consecutive N or N′ is joined to the next N or N′ by a covalent bond;
wherein Z and Z′ may be present or absent, but if present is independently 1-5 consecutive nucleotides covalently attached at the 3′ terminus of the strand in which it is present;
wherein z″ may be present or absent, but if present is a capping moiety covalently attached at the 5′ terminus of (N′)y;
wherein x=18 to 27;
wherein y=18 to 27;
wherein (N)x comprises modified and unmodified ribonucleotides, each modified ribonucleotide having a 2′-O-methyl on its sugar, wherein N at the 3′ terminus of (N)x is a modified ribonucleotide, (N)x comprises at least five alternating modified ribonucleotides beginning at the 3′ end and at least nine modified ribonucleotides in total and each remaining N is an unmodified ribonucleotide;
wherein in (N′)y at least one unconventional moiety is present, which unconventional moiety may be an abasic ribose moiety, an abasic deoxyribose moiety, a modified or unmodified deoxyribonucleotide, a mirror nucleotide, and a nucleotide joined to an adjacent nucleotide by a 2′-5′ internucleotide phosphate bond; and
wherein the sequence of (N′)_{y} is a sequence substantially complementary to (N)x; and
wherein the sequence of (N)ₓ comprises an antisense sequence substantially identical to an antisense sequence set forth in any one of Tables A-AQ.

In some embodiments x=y=19. In other embodiments x=y=23. In some embodiments the at least one unconventional moiety is present at positions 15, 16, 17, or 18 in (N′)y. In some embodiments the unconventional moiety is selected from a mirror nucleotide, an abasic ribose moiety and an abasic deoxyribose moiety. In some preferred embodiments the unconventional moiety is a mirror nucleotide, preferably an L-DNA moiety. In some embodiments an L-DNA moiety is present at position 17, position 18 or positions 17 and 18.

In other embodiments the unconventional moiety is an abasic moiety. In various embodiments (N′)y comprises at least five abasic ribose moieties or abasic deoxyribose moieties.

In yet other embodiments (N′)y comprises at least five abasic ribose moieties or abasic deoxyribose moieties and at least one of N′ is an LNA.

In some embodiments (N)x comprises nine alternating modified ribonucleotides. In other embodiments of Structure (I) (N)x comprises nine alternating modified ribonucleotides further comprising a 2′O modified nucleotide at position 2. In some embodiments (N)x comprises 2′O Me modified ribonucleotides at the odd numbered positions 1, 3, 5, 7, 9, 11, 13, 15, 17, 19. In other embodiments (N)x further comprises a 2′O Me modified ribonucleotide at one or both of positions 2 and 18. In yet other embodiments (N)x comprises 2′O Me modified ribonucleotides at positions 2, 4, 6, 8, 11, 13, 15, 17, 19.

In various embodiments z″ is present and is selected from an abasic ribose moiety, a deoxyribose moiety; an inverted abasic ribose moiety, a deoxyribose moiety; C6-amino-Pi; a mirror nucleotide.

In another aspect the present invention provides a compound having Structure (J) set forth below:

5′(N)x-Z3′ (antisense strand) (J)

3′Z′—(N′)y-z″5′ (sense strand)

wherein each of N and N′ is a ribonucleotide which may be unmodified or modified, or an unconventional moiety;
wherein each of (N)x and (N′)y is an oligonucleotide in which each consecutive N or N′ is joined to the next N or N′ by a covalent bond;
wherein Z and Z′ may be present or absent, but if present is independently 1-5 consecutive nucleotides covalently attached at the 3′ terminus of the strand in which it is present;
wherein z″ may be present or absent but if present is a capping moiety covalently attached at the 5′ terminus of (N′)y;
wherein x=18 to 27;
wherein y=18 to 27;
wherein (N)x comprises modified or unmodified ribonucleotides, and optionally at least one unconventional moiety;
wherein in (N′)y at least one unconventional moiety is present, which unconventional moiety may be an abasic ribose moiety, an abasic deoxyribose moiety, a modified or unmodified deoxyribonucleotide, a mirror nucleotide, a non-base pairing nucleotide analog or a nucleotide joined to an adjacent nucleotide by a 2′-5′ internucleotide phosphate bond; and
wherein the sequence of (N′)_{y} is a sequence substantially complementary to (N)x; and
wherein the sequence of (N)ₓ comprises an antisense sequence substantially identical to an antisense sequence set forth in any one of Tables A-AQ.

In some embodiments x=y=19. In other embodiments x=y=23. In some preferred embodiments (N)x comprises modified and unmodified ribonucleotides, and at least one unconventional moiety.

In some embodiments in (N)x the N at the 3′ terminus is a modified ribonucleotide and (N)x comprises at least 8 modified ribonucleotides. In other embodiments at least 5 of the at least 8 modified ribonucleotides are alternating beginning at the 3′ end. In some embodiments (N)x comprises an abasic moiety in one of positions 5, 6, 7, 8, 9, 10, 11, 12, 13, 14 or 15.

In some embodiments the at least one unconventional moiety in (N′)y is present at positions 15, 16, 17, or 18. In some embodiments the unconventional moiety is selected from a mirror nucleotide, an abasic ribose moiety and an abasic deoxyribose moiety. In some preferred embodiments the unconventional moiety is a mirror nucleotide, preferably an L-DNA moiety. In some embodiments an L-DNA moiety is present at position 17, position 18 or positions 17 and 18. In other embodiments the at least one unconventional moiety in (N′)y is an abasic ribose moiety or an abasic deoxyribose moiety.

In yet another aspect the present invention provides a compound having Structure (K) set forth below:

5′(N)ₓ—Z3′ (antisense strand) (K)

3′Z′—(N′)_{y}-z″5′ (sense strand)

wherein each of N and N′ is a ribonucleotide which may be unmodified or modified, or an unconventional moiety;
wherein each of (N)x and (N′)y is an oligonucleotide in which each consecutive N or N′ is joined to the next N or N′ by a covalent bond;
wherein Z and Z′ may be present or absent, but if present is independently 1-5 consecutive nucleotides covalently attached at the 3′ terminus of the strand in which it is present;
wherein z″ may be present or absent but if present is a capping moiety covalently attached at the 5′ terminus of (N′)y;
wherein x=18 to 27;
wherein y=18 to 27;
wherein (N)x comprises a combination of modified or unmodified ribonucleotides and unconventional moieties, any modified ribonucleotide having a 2′-O-methyl on its sugar;
wherein (N′)y comprises modified or unmodified ribonucleotides and optionally an unconventional moiety, any modified ribonucleotide having a 2′OMe on its sugar;
wherein the sequence of (N′)_{y} is a sequence substantially complementary to (N)x; and
wherein the sequence of (N)ₓ comprises an antisense sequence substantially identical to an antisense sequence set forth in any one of Tables A-AQ.

In some embodiments x=y=19. In other embodiments x=y=23. In some preferred embodiments the at least one preferred one unconventional moiety is present in (N)x and is an abasic ribose moiety or an abasic deoxyribose moiety. In other embodiments the at least one unconventional moiety is present in (N)x and is a non-base pairing nucleotide analog. In various embodiments (N′)y comprises unmodified ribonucleotides. In some embodiments (N)x comprises at least five abasic ribose moieties or abasic deoxyribose moieties or a combination thereof. In certain embodiments (N)x and/or (N′)y comprise modified ribonucleotides which do not base pair with corresponding modified or unmodified ribonucleotides in (N′)y and/or (N)x.

In various embodiments the present invention provides an siRNA set forth in Structure (L):

5′(N)ₓ—Z3′ (antisense strand) (L)

3′Z′—(N′)_{y}5′ (sense strand)

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein Z and Z′ are absent;
wherein x=y=19;
wherein in (N′)y the nucleotide in at least one of positions 15, 16, 17, 18 and 19 comprises a nucleotide selected from an abasic unconventional moiety, a mirror nucleotide, a deoxyribonucleotide and a nucleotide joined to an adjacent nucleotide by a 2′-5′ internucleotide bond;
wherein (N)x comprises alternating modified ribonucleotides and unmodified ribonucleotides each modified ribonucleotide being modified so as to have a 2′-O-methyl on its sugar and the ribonucleotide located at the middle position of (N)x being modified or unmodified, preferably unmodified; and
wherein the sequence of (N′)_{y} is a sequence substantially complementary to (N)x; and
wherein the sequence of (N)ₓ comprises an antisense sequence substantially identical to an antisense sequence set forth in any one of Tables A-AQ.

In some embodiments of Structure (L), in (N′)y the nucleotide in one or both of positions 17 and 18 comprises a modified nucleotide selected from an abasic unconventional moiety, a mirror nucleotide and a nucleotide joined to an adjacent nucleotide by a 2′-5′ internucleotide bond. In some embodiments the mirror nucleotide is selected from L-DNA and L-RNA. In various embodiments the mirror nucleotide is L-DNA.

In various embodiments (N′)y comprises a modified nucleotide at position 15 wherein the modified nucleotide is selected from a mirror nucleotide and a deoxyribonucleotide.

In certain embodiments (N′)y further comprises a modified nucleotide or pseudo nucleotide at position 2 wherein the pseudo nucleotide may be an abasic unconventional moiety and the modified nucleotide is optionally a mirror nucleotide.

In various embodiments the antisense strand (N)x comprises 2′O-Me modified ribonucleotides at the odd numbered positions (5′ to 3′; positions 1, 3, 5, 7, 9, 11, 13, 15, 17, 19). In some embodiments (N)x further comprises 2′O-Me modified ribonucleotides at one or both positions 2 and 18. In other embodiments (N)x comprises 2′O Me modified ribonucleotides at positions 2, 4, 6, 8, 11, 13, 15, 17, 19.

Other embodiments of Structures (L) are envisaged wherein x=y=21 or wherein x=y=23; in these embodiments the modifications for (N′)y discussed above instead of being in positions 17 and 18 are in positions 19 and 20 for 21-mer oligonucleotide and 21 and 22 for 23 mer oligonucleotide; similarly the modifications in positions 15, 16, 17, 18 or 19 are in positions 17, 18, 19, 20 or 21 for the 21-mer oligonucleotide and positions 19, 20, 21, 22, or 23 for the 23-mer oligonucleotide. The 2′O Me modifications on the antisense strand are similarly adjusted. In some embodiments (N)x comprises 2′O Me modified ribonucleotides at the odd numbered positions (5′ to 3′; positions 1, 3, 5, 7, 9, 12, 14, 16, 18, 20 for the 21 mer oligonucleotide [nucleotide at position 11 unmodified] and 1, 3, 5, 7, 9, 11, 13, 15, 17, 19, 21, 23 for the 23 mer oligonucleotide [nucleotide at position 12 unmodified]. In other embodiments (N)x comprises 2′O Me modified ribonucleotides at positions 2, 4, 6, 8, 10, 12, 14, 16, 18, 20 [nucleotide at position 11 unmodified for the 21 mer oligonucleotide and at positions 2, 4, 6, 8, 10, 13, 15, 17, 19, 21, 23 for the 23 mer oligonucleotide [nucleotide at position 12 unmodified].

In some embodiments (N′)y further comprises a 5′ terminal cap nucleotide. In various embodiments the terminal cap moiety is selected from an abasic unconventional moiety, an inverted abasic unconventional moiety, an L-DNA nucleotide, and a C6-imine phosphate (C6 amino linker with phosphate at terminus).

In other embodiments the present invention provides a compound having Structure (M) set forth below:

5′(N)ₓ—Z3′ (antisense strand) (M)

3′Z′—(N′)_{y}5′ (sense strand)

wherein each of N and N′ is selected from a pseudo-nucleotide and a nucleotide;
wherein each nucleotide is selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein Z and Z′ are absent;
wherein x=18 to 27;
wherein y=18 to 27;
wherein the sequence of (N′)_{y} is a sequence substantially complementary to (N)x; and
wherein the sequence of (N)ₓ comprises an antisense sequence substantially identical to an antisense sequence set forth in any one of Tables A-AQ.

In other embodiments the present invention provides a double stranded compound having Structure (N) set forth below:

5′(N)ₓ—Z3′ (antisense strand) (N)

3′Z′—(N′)_{y}5′ (sense strand)

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein Z and Z′ are absent;
wherein each of x and y is an integer between 18 and 40;
wherein the sequence of (N′)_{y} is a sequence substantially complementary to (N)x; and
wherein the sequence of (N)ₓ comprises an antisense sequence substantially identical to an antisense sequence set forth in any one of Tables A-AQ;
wherein (N)x, (N′)y or (N)x and (N′)y comprise non base-pairing modified nucleotides such that (N)x and (N′)y form less than 15 base pairs in the double stranded compound.

In other embodiments the present invention provides a compound having Structure (O) set forth below:

5′(N)ₓ—Z3′ (antisense strand) (O)

3′Z′—(N′)_{y}5′ (sense strand)

wherein each of N is a nucleotide selected from an unmodified ribonucleotide, a modified ribonucleotide, an unmodified deoxyribonucleotide and a modified deoxyribonucleotide;
wherein each of N′ is a nucleotide analog selected from a six membered sugar nucleotide, seven membered sugar nucleotide, morpholino moiety, peptide nucleic acid and combinations thereof;
wherein each of (N)ₓ and (N′)_{y} is an oligonucleotide in which each consecutive N or N′ is joined to the next N or N′ by a covalent bond;
wherein Z and Z′ are absent;
wherein each of x and y is an integer between 18 and 40;
wherein the sequence of (N′)_{y} is a sequence substantially complementary to (N)x; and
wherein the sequence of (N)ₓ comprises an antisense sequence substantially identical to an antisense sequence set forth in any one of Tables A-AQ.

In other embodiments the present invention provides a compound having Structure (P) set forth below:

5′(N)ₓ—Z3′ (antisense strand) (P)

3′Z′—(N′)_{y}5′ (sense strand)

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein Z and Z′ are absent;
wherein each of x and y is an integer between 18 and 40;
wherein one of N or N′ in an internal position of (N)x or (N′)y or one or more of N or N′ at a terminal position of (N)x or (N′)y comprises an abasic moiety or a 2′ modified nucleotide;
wherein the sequence of (N′)_{y} is a sequence substantially complementary to (N)x; and
wherein the sequence of (N)ₓ comprises an antisense sequence substantially identical to an antisense sequence set forth in any one of Tables A-AQ.

In various embodiments (N′)y comprises a modified nucleotide at position 15 wherein the modified nucleotide is selected from a mirror nucleotide and a deoxyribonucleotide.

In certain embodiments (N′)y further comprises a modified nucleotide at position 2 wherein the modified nucleotide is selected from a mirror nucleotide and an abasic unconventional moiety.

In various embodiments the antisense strand (N)x comprises 2′O-Me modified ribonucleotides at the odd numbered positions (5′ to 3′; positions 1, 3, 5, 7, 9, 11, 13, 15, 17, 19). In some embodiments (N)ₓ further comprises 2′O-Me modified ribonucleotides at one or both positions 2 and 18. In other embodiments (N)x comprises 2′O Me modified ribonucleotides at positions 2, 4, 6, 8, 11, 13, 15, 17, 19.

An additional novel molecule provided by the present invention is an oligonucleotide comprising consecutive nucleotides wherein a first segment of such nucleotides encode a first inhibitory RNA molecule, a second segment of such nucleotides encode a second inhibitory RNA molecule, and a third segment of such nucleotides encode a third inhibitory RNA molecule. Each of the first, the second and the third segment may comprise one strand of a double stranded RNA and the first, second and third segments may be joined together by a linker. Further, the oligonucleotide may comprise three double stranded segments joined together by one or more linker.

Thus, one molecule provided by the present invention is an oligonucleotide comprising consecutive nucleotides which encode three inhibitory RNA molecules; said oligonucleotide may possess a triple stranded structure, such that three double stranded arms are linked together by one or more linker, such as any of the linkers presented hereinabove. This molecule forms a “star”-like structure, and may also be referred to herein as RNAstar.

Said triple-stranded oligonucleotide may be an oligoribonucleotide having the general structure:

| | | | | |
|---|---|---|---|---|
| 5′ | Oligo1 (sense) | LINKER A | Oligo2 (sense) | 3′ |
| 3′ | Oligo1 (antisense) | LINKER B | Oligo3 (sense) | 5′ |
| 3′ | Oligo3 (antisense) | LINKER C | Oligo2 (antisense) | 5′ |

or

| | | | | |
|---|---|---|---|---|
| 5′ | Oligo1 (sense) | LINKER A | Oligo2 (antisense) | 3′ |
| 3′ | Oligo1 (antisense) | LINKER B | Oligo3 (sense) | 5′ |
| 3′ | Oligo3 (antisense) | LINKER C | Oligo2 (sense) | 5′ |

or

| | | | | |
|---|---|---|---|---|
| 5′ | Oligo1 (sense) | LINKER A | Oligo3 (antisense) | 3′ |
| 3′ | Oligo1 (antisense) | LINKER B | Oligo2 (sense) | 5′ |
| 5′ | Oligo3 (sense) | LINKER C | Oligo2 (antisense) | 3′ |

wherein one or more of linker A, linker B or linker C is present; any combination of two or more oligonucleotides and one or more of linkers A-C is possible, so long as the polarity of the strands and the general structure of the molecule remains. Further, if two or more of linkers A-C are present, they may be identical or different.

Thus, a triple-armed structure is formed, wherein each arm comprises a sense strand and complementary antisense strand (i.e. Oligo1 antisense base pairs to Oligo1 sense etc.). The triple armed structure may be triple stranded, whereby each arm possesses base pairing.

Further, the above triple stranded structure may have a gap instead of a linker in one or more of the strands. Such a molecule with one gap is technically quadruple stranded and not triple stranded; inserting additional gaps or nicks will lead to the molecule having additional strands. Preliminary results obtained by the inventors of the present invention indicate that said gapped molecules are more active in inhibiting certain target genes than the similar but non-gapped molecules. This may also be the case for nicked molecules.

According to one preferred embodiment of the invention, the antisense and the sense strands of the siRNA are phosphorylated only at the 3′-terminus and not at the 5′-terminus. According to another preferred embodiment of the invention, the antisense and the sense strands are non-phosphorylated. According to yet another preferred embodiment of the invention, the 5′ most ribonucleotide in the sense strand is modified to abolish any possibility of in vivo 5′-phosphorylation.

The invention further provides a vector capable of expressing any of the aforementioned oligoribonucleotides in unmodified form in a cell after which appropriate modification may be made. In preferred embodiment the cell is a mammalian cell, preferably a human cell.

### Pharmaceutical Compositions

While it may be possible for the compounds of the present invention to be administered as the raw chemical, it is preferable to present them as a pharmaceutical composition. Accordingly the present invention provides a pharmaceutical composition comprising one or more of the compounds of the invention; and a pharmaceutically acceptable carrier. This composition may comprise a mixture of two or more different siRNAs.

The invention further provides a pharmaceutical composition comprising at least one compound of the invention covalently or non-covalently bound to one or more compounds of the invention in an amount effective to inhibit the target genes of the present invention; and a pharmaceutically acceptable carrier. The compound may be processed intracellularly by endogenous cellular complexes to produce one or more oligoribonucleotides of the invention.

The invention further provides a pharmaceutical composition comprising a pharmaceutically acceptable carrier and one or more of the compounds of the invention in an amount effective to inhibit expression in a cell of a human target gene of the present invention, the compound comprising a sequence which is substantially complementary to the sequence of (N)ₓ.

Substantially complementary refers to complementarity of greater than about 84%, to another sequence. For example in a duplex region consisting of 19 base pairs one mismatch results in 94.7% complementarity, two mismatches results in about 89.5% complementarity and 3 mismatches results in about 84.2% complementarity, rendering the duplex region substantially complementary. Accordingly substantially identical refers to identity of greater than about 84%, to another sequence.

Additionally, the invention provides a method of inhibiting the expression of the target genes of the present invention by at least 40%, preferably by 50%, 60% or 70%, more preferably by 75%, 80% or 90% as compared to a control comprising contacting an mRNA transcript of the target gene of the present invention with one or more of the compounds of the invention.

In one embodiment the oligoribonucleotide is inhibiting one or more of the target genes of the present invention, whereby the inhibition is selected from the group comprising inhibition of gene function, inhibition of polypeptide and inhibition of mRNA expression.

In one embodiment the compound inhibits the target polypeptide, whereby the inhibition is selected from the group comprising inhibition of function (which may be examined by an enzymatic assay or a binding assay with a known interactor of the native gene/polypeptide, inter alia), inhibition of protein (which may be examined by Western blotting, ELISA or immuno-precipitation, inter alia) and inhibition of mRNA expression (which may be examined by Northern blotting, quantitative RT-PCR, in-situ hybridisation or microarray hybridisation, inter alia).

In additional embodiments the invention provides a method of treating a subject suffering from a disease accompanied by an elevated level of the target genes of the present invention, the method comprising administering to the subject a compound of the invention in a therapeutically effective dose thereby treating the subject.

More particularly, the invention provides an oligoribonucleotide wherein one strand comprises consecutive nucleotides having, from 5′ to 3′, the sequence set forth in Tables A-AQ, or a homolog thereof wherein in up to two of the ribonucleotides in each terminal region is altered.

It will be understood by one skilled in the art that given the potential length of the nucleic acid according to the present invention and particularly of the individual strands forming such nucleic acid according to the present invention, some shifts relative to the coding sequence of the target genes of the present invention to each side is possible, whereby such shifts can be up to 1, 2, 3, 4, 5 and 6 nucleotides in both directions, and whereby the thus generated double-stranded nucleic acid molecules shall also be within the present invention.

### Delivery

The siRNA molecules of the present invention may be delivered to the target tissue by direct application of the naked molecules prepared with a carrier or a diluent.

The term “naked siRNA” refers to siRNA molecules that are free from any delivery vehicle that acts to assist, promote or facilitate entry into the cell, including viral sequences, viral particles, liposome formulations, lipofectin or precipitating agents and the like. For example, siRNA in PBS is “naked siRNA”.

However, in some embodiments the siRNA molecules of the invention are delivered in liposome formulations and lipofectin formulations and the like and can be prepared by methods well known to those skilled in the art. Such methods are described, for example, in U.S. Pat. Nos. 5,593,972, 5,589,466, and 5,580,859, which are herein incorporated by reference.

Delivery systems aimed specifically at the enhanced and improved delivery of siRNA into mammalian cells have been developed, (see, for example, Shen et al., FEBS Let. 2003, 539:111-114; Xia et al., Nat. Biotech. 2002, 20:1006-1010; Reich et al., Mol. Vision 2003, 9: 210-216; Sorensen et al., J. Mol. Biol. 2003. 327: 761-766; Lewis et al., Nat. Gen. 2002, 32: 107-108 and Simeoni et al., NAR 2003, 31, 11: 2717-2724). siRNA has recently been successfully used for inhibition of gene expression in primates (see for example, Tolentino et al., Retina 24(4):660).

The pharmaceutically acceptable carriers, solvents, diluents, excipients, adjuvants and vehicles as well as implant carriers generally refer to inert, non-toxic solid or liquid fillers, diluents or encapsulating material not reacting with the active ingredients of the invention and they include liposomes and microspheres. Examples of delivery systems useful in the present invention include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art. In one specific embodiment of this invention topical and transdermal formulations may be selected. The siRNAs or pharmaceutical compositions of the present invention are administered and dosed in accordance with good medical practice, taking into account the clinical condition of the individual subject, the disease to be treated, the site and method of administration, scheduling of administration, patient age, sex, body weight and other factors known to medical practitioners.

In another embodiment the administration comprises topical or local administration such as via eye drops or eardrops. In a non-limiting example, siRNA compounds that target SOX9, ASPP1, CTSD, CAPNS1, are useful in treating a subject suffering from a hearing loss or in preventing hearing loss in a subject receiving chemotherapy wherein the siRNA compounds are delivered to the ear via transtympanic injection or via eardrops.

In a non-limiting example, siRNA compounds that target SOX9, ASPP1, CTSD, CAPNS1, FAS and FAS ligand are useful in treating a subject suffering from a neurodegenerative disease (AD, ALS) and the siRNA compounds are delivered to the CNS by intranasal administration.

In particular embodiments the SOX9, ASPP1, CTSD, CAPNS1, FAS and FAS ligand siRNA compounds are formulated as eye drops for administration to the surface of the eye. In other embodiments the siRNA compounds are administered to the lung by inhalation.

In addition, in certain embodiments the compositions for use in the novel treatments of the present invention may be formed as aerosols, for example for intranasal administration.

The “therapeutically effective dose” for purposes herein is thus determined by such considerations as are known in the art. The dose must be effective to achieve improvement including but not limited to improved survival rate or more rapid recovery, or improvement or elimination of symptoms and other indicators as are selected as appropriate measures by those skilled in the art.

In general, the active dose of compound for humans is in the range of from 1 ng/kg to about 20-100 mg/kg body weight per day, preferably about 0.01 mg to about 2-10 mg/kg body weight per day, in a regimen of one dose per day or twice or three or more times per day for a period of 1-4 weeks or longer. The compounds of the present invention can be administered by any of the conventional routes of administration. It should be noted that the compound can be administered as the compound or as pharmaceutically acceptable salt and can be administered alone or as an active ingredient in combination with pharmaceutically acceptable carriers, solvents, diluents, excipients, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. For treating spinal-cord injury, local delivery of the siRNA molecules is preferable for example by using direct injection to the spinal cord or by using intrathecal delivery (such as using Alzet pump). Liquid forms may be prepared for injection, the term including subcutaneous, transdermal, intravenous, intramuscular, intrathecal, and other parental routes of administration. The liquid compositions include aqueous solutions, with and without organic co-solvents, aqueous or oil suspensions, emulsions with edible oils, as well as similar pharmaceutical vehicles. In a particular embodiment, the administration comprises intravenous administration. In another embodiment the administration comprises topical or local administration. In addition, in certain embodiments the compositions for use in the novel treatments of the present invention may be formed as aerosols, for example for intranasal administration. In certain embodiments, oral compositions (such as tablets, suspensions, solutions) may be effective for local delivery to the oral cavity such as oral composition suitable for mouthwash for the treatment of oral mucositis.

### Methods of Treatment

In another aspect, the present invention relates to a method for the treatment of a subject in need of treatment for a disease or disorder associated with the abnormal expression of the target genes of Table 1, comprising administering to the subject an amount of an inhibitor, which reduces or inhibits expression of these genes.

In preferred embodiments the subject being treated is a warm-blooded animal and, in particular, mammals including human.

The methods of the invention comprise administering to the subject one or more inhibitory compounds which down-regulate expression of the target genes of Table 1; and in particular siRNA in a therapeutically effective dose so as to thereby treat the subject.

In various embodiments the inhibitor is selected from the group consisting of siRNA, shRNA, an aptamer, an antisense molecule, miRNA, and a ribozyme. In the presently preferred embodiments the inhibitor is siRNA.

The term “treatment” refers to both therapeutic treatment and prophylactic or preventative measures, wherein the object is to prevent or slow down (lessen) related disorders as listed above. Those in need of treatment include those already experiencing the disease or condition, those prone to having the disease or condition, and those in which the disease or condition is to be prevented. The compounds of the invention may be administered before, during or subsequent to the onset of the disease or condition or symptoms associated therewith. In cases where treatment is for the purpose of prevention, then the present invention relates to a method for delaying the onset of or averting the development of the disease or disorder.

The present invention relates to the use of compounds which down-regulate the expression of the target genes of the invention particularly to novel small interfering RNAs (siRNAs), in the treatment of the following diseases or conditions in which inhibition of the expression of the target genes is beneficial.

Specifically, inhibition of SOX9, CTSD, CAPNS1 genes by siRNA molecules is beneficial in the treatment or prevention of spinal-cord injury or any other related injury. The compounds of the invention are used for treating or preventing the damage caused by spinal-cord injury especially spinal cord trauma caused by motor vehicle accidents, falls, sports injuries, industrial accidents, gunshot wounds, spinal cord trauma caused by spine weakening (such as from rheumatoid arthritis or osteoporosis) or if the spinal canal protecting the spinal cord has become too narrow (spinal stenosis) due to the normal aging process, direct damage that occur when the spinal cord is pulled, pressed sideways, or compressed, damage to the spinal-cord following bleeding, fluid accumulation, and swelling inside the spinal cord or outside the spinal cord (but within the spinal canal). The compounds of the invention are also used for treating or preventing the damage caused by spinal-cord injury due to disease such as polio or spina bifida.

Inhibition of FAS and FAS ligand genes by siRNA molecules is beneficial in the treatment of dry eye syndromes or any other related lacrimal gland dysfunctions. There are two major types of dry eye syndromes: aqueous-deficient dry eye due to lacrimal gland diseases and evaporative dry eye mainly due to meibomian gland diseases. Disorders of the lacrimal gland include for example Sjogren's syndrome (focal lymphocytic infiltration of the lacrimal and salivary glands), Sarcoidosis (non-caseating granulomas in multiple organs including lacrimal glands), Chronic graft-versus-host disease (prominent fibrosis and an increase in stromal fibroblasts in the lacrimal gland) and decrease in lacrimal gland secretion in aging.

The compounds of the invention are used for treating acute renal failure, in particular acute renal failure due to ischemia in post surgical patients, and acute renal failure due to chemotherapy treatment such as cisplatin administration or sepsis-associated acute renal failure. Another use of the compounds of the invention is for the prevention of acute renal failure in high-risk patients undergoing major cardiac surgery or vascular surgery. The patients at high-risk of developing acute renal failure can be identified using various scoring methods such as the Cleveland Clinic algorithm or that developed by US Academic Hospitals (QMMI) and by Veterans' Administration (CICSS). Other uses of the compounds of the invention are for the prevention of ischemic acute renal failure in kidney transplant patients or for the prevention of toxic acute renal failure in patients receiving chemotherapy.

In another preferred embodiment, the compounds of the invention are used for treating or preventing the damage caused by nephrotoxins such as diuretics, β-blockers, vasodilator agents, ACE inhibitors, cyclosporin, aminoglycoside antibiotics (e.g. gentamicin), amphotericin B, cisplatin, radiocontrast media, immunoglobulins, mannitol, NSAIDs (eg aspirin, ibuprofen, diclofenac), cyclophosphamide, methotrexate, aciclovir, polyethylene glycol, β-lactam antibiotics, vancomycin, rifampicin, sulphonamides, ciprofloxacin, ranitidine, cimetidine, furosemide, thiazides, phenyloin, penicillamine, lithium salts, fluoride, demeclocycline, foscarnet, aristolochic acid.

The compounds of the invention are also used for treating glaucoma. Main types of glaucoma are primary open angle glaucoma (POAG), angle closure glaucoma, normal tension glaucoma and pediatric glaucoma. These are marked by an increase of intraocular pressure (IOP), or pressure inside the eye. When optic nerve damage has occurred despite a normal IOP, this is called normal tension glaucoma. Secondary glaucoma refers to any case in which another disease causes or contributes to increased eye pressure, resulting in optic nerve damage and vision loss.

The methods of the invention are applied to various conditions of acute hearing loss. Without being bound by theory, the hearing loss may be due to apoptotic inner ear hair cell damage or loss, wherein the damage or loss is caused by infection, mechanical injury, loud sound, aging (presbycusis), or chemical-induced ototoxicity. Ototoxins include therapeutic drugs including antineoplastic agents, salicylates, quinines, and aminoglycoside antibiotics, contaminants in foods or medicinals, and environmental or industrial pollutants. Typically, treatment is performed to prevent or reduce ototoxicity, especially resulting from or expected to result from administration of therapeutic drugs. Preferably a therapeutically effective composition is given immediately after the exposure to prevent or reduce the ototoxic effect. More preferably, treatment is provided prophylactically, either by administration of the composition prior to or concomitantly with the ototoxic pharmaceutical or the exposure to the ototoxin.

By “ototoxin” in the context of the present invention is meant a substance that through its chemical action injures, impairs or inhibits the activity of the sound receptors component of the nervous system related to hearing, which in turn impairs hearing (and/or balance). In the context of the present invention, ototoxicity includes a deleterious effect on the inner ear hair cells. Ototoxic agents that cause hearing impairments include, but are not limited to, antineoplastic agents such as vincristine, vinblastine, cisplatin and cisplatin-like compounds, taxol and taxol-like compounds, dideoxy-compounds, e.g., dideoxyinosine; alcohol; metals; industrial toxins involved in occupational or environmental exposure; contaminants of food or medicinals; and over-doses of vitamins or therapeutic drugs, e.g., antibiotics such as penicillin or chloramphenicol, and megadoses of vitamins A, D, or B6, salicylates, quinines, loop diuretics, and phosphodiesterase type 5 (PDE5) inhibitors such as sildenafil citrate (Viagra®).

Hearing loss may be due to end-organ lesions involving inner ear hair cells, e.g., acoustic trauma, viral endolymphatic labyrinthitis, Meniere's disease. Hearing impairments include tinnitus, which is a perception of sound in the absence of an acoustic stimulus, and may be intermittent or continuous, wherein there is diagnosed a sensorineural loss. Hearing loss may be due to bacterial or viral infection, such as in herpes zoster oticus, purulent labyrinthitis arising from acute otitis media, purulent meningitis, chronic otitis media, sudden deafness including that of viral origin, e.g., viral endolymphatic labyrinthitis caused by viruses including mumps, measles, influenza, chicken pox, mononucleosis and adenoviruses. The hearing loss can be congenital, such as that caused by rubella, anoxia during birth, bleeding into the inner ear due to trauma during delivery, ototoxic drugs administered to the mother, erythroblastosis fetalis, and hereditary conditions including Waardenburg's syndrome and Hurler's syndrome.

The hearing loss can be noise-induced, generally due to a noise greater than 85 decibels (db) that damages the inner ear. In a particular aspect of the invention, the hearing loss is caused by an ototoxic drug that effects the auditory portion of the inner ear, particularly inner ear hair cells. Incorporated herein by reference are chapters 196, 197, 198 and 199 of The Merck Manual of Diagnosis and Therapy, 14th Edition, (1982), Merck Sharp & Dome Research Laboratories, N.J. and corresponding chapters in the most recent 16th edition, including Chapters 207 and 210) relating to description and diagnosis of hearing and balance impairments.

Methods, molecules and compositions which inhibit the target genes of the invention are discussed herein at length, and any of said molecules and/or compositions may be beneficially employed in the treatment of a subject suffering from any of said conditions. Preferred oligomer sequences useful in the preparation of siRNA directed to selected target genes are listed in Tables A-AQ.

The present invention further relates to methods for treating or preventing the incidence or severity of various diseases or conditions in a subject in need thereof wherein the disease or condition and/or symptoms associated therewith is selected from the group consisting of a neurodegenerative disease or disorder including spinal cord injury, Alzheimer's disease (AD) and Amyotrophic Lateral Sclerosis (ALS); acute renal failure (ARF); hearing loss; an ophthalmic disease including glaucoma and ischemic optic neuropathy (ION); a respiratory disease including acute respiratory distress syndrome (ARDS), chronic obstructive pulmonary disease (COPD) and other acute lung and respiratory injuries; injury (e.g. ischemia-reperfusion injury) in organ transplant including lung, kidney, bone marrow, heart, pancreas, cornea or liver transplantation; nephrotoxicity, pressure sores, dry eye syndrome or oral mucositis.

In other embodiments the compounds and methods of the invention are useful for treating or preventing the incidence or severity of other diseases and conditions in a patient. These diseases and conditions include stroke and stroke-like situations (e.g. cerebral, renal, cardiac failure), neuronal cell death, brain injuries with or without reperfusion issues, chronic degenerative diseases e.g. neurodegenerative disease including Alzheimer's disease, Huntington's disease, Parkinson's disease, multiple sclerosis, amyotrophic lateral sclerosis, spinobulbar atrophy, prion disease, and apoptosis resulting from traumatic brain injury (TBI).

The compounds and methods of the invention are directed to providing neuroprotection, or to provide cerebroprotection, or to prevent and/or treat cytotoxic T cell and natural killer cell-mediated apoptosis associated with autoimmune disease and transplant rejection, to prevent cell death of cardiac cells including heart failure, cardiomyopathy, viral infection or bacterial infection of heart, myocardial ischemia, myocardial infarct, and myocardial ischemia, coronary artery by-pass graft, to prevent and/or treat mitochondrial drug toxicity e.g. as a result of chemotherapy or HIV therapy, to prevent cell death during viral infection or bacterial infection, or to prevent and/or treat inflammation or inflammatory diseases, inflammatory bowel disease, sepsis and septic shock, or to prevent cell death from follicle to ovocyte stages, from ovocyte to mature egg stages and sperm (for example, methods of freezing and transplanting ovarian tissue, artificial fertilization), or to preserve fertility in mammals after chemotherapy, in particular human mammals, or to prevent and/or treat, macular degeneration, or to prevent and/or treat acute hepatitis, chronic active hepatitis, hepatitis-B, and hepatitis-C, or to prevent hair loss, (e.g. hair loss due-to male-pattern baldness, or hair loss due to radiation, chemotherapy or emotional stress), or to treat or ameliorate skin damage whereby the skin damage may be due to exposure to high levels of radiation, heat, chemicals, sun, or to burns and autoimmune diseases), or to prevent cell death of bone marrow cells in myelodysplastic syndromes (MDS), to treat pancreatitis, to treat rheumatoid arthritis, psoriasis, glomerulonephritis, atherosclerosis, and graft versus host disease (GVHD), or to treat retinal pericyte apoptosis, retinal damages resulting from ischemia, diabetic retinopathy, or to treat any disease states associated with an increase of apoptotic cell death.

Details of certain indications in which the compounds of the present invention are useful as therapeutics are provided below.

### Respiratory Diseases

### Emphysema and COPD

Among the mechanisms that underlie lung destruction in emphysema, excessive formation of reactive oxygen species (ROS) should be first of all mentioned. It is well established that prooxidant/antioxidant imbalance exists in the blood and in the lung tissue of smokers (Hulea S A, et al: 1995. J Environ Pathol Toxicol Oncol. 14(3-4):173-80.; Rahman I, MacNee W. 1999. Am J. Physiol 277(6 Pt 1):L1067-88.; MacNee W. 2000 Chest. 117(5 Suppl 1):303S-17S; Marwick J A, et al., 2002. Ann NY Acad Sci. 973:278-83; Aoshiba K, et al., 2003. Inhal Toxicol. (10): 1029-38; Dekhuijzen P N. 2004. Eur Respir J. 23(4):629-36; Tuder R M, et al., 2003. Am J Respir Cell Mol Biol, 29:88-97). After one hour exposure of mice to CS, there is a dramatic increase of 8-hydroxy-2′-deoxyguanosine (8-OHdG) in the alveolar epithelial cells, particularly of type II (see Inhal Toxicol. 2003 15(10): 1029-38, above).

Overproduced reactive oxygen species are known for their cytotoxic activity, which stems from a direct DNA damaging effect and from the activation of apoptotic signal transduction pathways (Takahashi et al., 2004. Brain Res Bull. 62(6):497-504; Taniyama Y, Griendling K K. 2003. Hypertension. 42(6):1075-81; Higuchi Y. 2003. Biochem Pharmacol. 66(8):1527-35; Punj V, Chakrabarty A M. 2003. Cell Microbiol. (4):225-31.; Ueda et al., 2002 Antioxid Redox Signal. 4(3):405-14).

### Acute Respiratory Distress Syndrome

Acute respiratory distress syndrome (ARDS), also known as respiratory distress syndrome (RDS) or adult respiratory distress syndrome (in contrast with infant respiratory distress syndrome, IRDS) is a serious reaction to various forms of injuries to the lung. This is the most important disorder resulting in increased permeability pulmonary edema.

ARDS is a severe lung disease caused by a variety of direct and indirect insults. It is characterized by inflammation of the lung parenchyma leading to impaired gas exchange with concomitant systemic release of inflammatory mediators causing inflammation, hypoxemia and frequently resulting in multiple organ failure. This condition is life threatening, usually requiring mechanical ventilation and admission to an intensive care unit. A less severe form is called acute lung injury (ALI).

### Kidney Diseases and Disorders

### Acute Renal Failure

Acute renal failure (ARF) is a clinical syndrome characterized by rapid deterioration of renal function that occurs within days. The principal feature of ARF is an abrupt decline in glomerular filtration rate (GFR), resulting in the retention of nitrogenous wastes (urea, creatinine). Worldwide, severe ARF occurs in about 170-200 per million population annually. To date, there is no specific treatment for established ARF. Several drugs have been found to ameliorate toxic and ischemic experimental ARF, as manifested by lower serum creatinine levels, reduced histological damage and faster recovery of renal function in different animal models. These include anti-oxidants, calcium channel blockers, diuretics, vasoactive substances, growth factors, anti-inflammatory agents and more. However, the drugs tested in clinical trials showed no benefit, and their use in clinical ARF has not been approved.

In the majority of hospitalized ARF patients, ARF is caused by acute tubular necrosis (ATN), which results from ischemic and/or nephrotoxic insults. Renal hypoperfusion is caused by hypovolemic, cardiogenic and septic shock, by administration of vasoconstrictive drugs or renovascular injury. Nephrotoxins include exogenous toxins such as contrast media, aminoglycosides and cisplatin and cisplatin-like compounds as well as endogenous toxin such as myoglobin. Recent studies, however, support the theory that apoptosis in renal tissues is prominent in most human cases of ARF. The principal site of apoptotic cell death is the distal nephron. During the initial phase of ischemic injury, loss of integrity of the actin cytoskeleton leads to flattening of the epithelium, with loss of the brush border, loss of focal cell contacts, and subsequent disengagement of the cell from the underlying substratum. It has been suggested that apoptotic tubule cell death may be more predictive of functional changes than necrotic cell death (Komarov et al., Science 1999, 10; 285(5434):1733-7); Supavekin et al., Kidney Int. 2003, 63(5):1714-24).

In conclusion, currently there are no satisfactory modes of therapy for the prevention and/or treatment of acute renal failure, and there is a need therefore to develop novel compounds for this purpose.

### Microvascular Diseases of the Kidney

The kidney is involved in a number of discreet clinicopathologic conditions that affect systemic and renal microvasculature. Certain of these conditions are characterized by primary injury to endothelial cells, such as: Hemolytic-uremic syndrome (HUS) and thrombotic thrombocytopenic purpura (TTP) and Radiation nephritis—The long-term consequences of renal irradiation in excess of 2500 rad.

Taken as a group, diseases that cause transient or permanent occlusion of renal microvasculature uniformly result in disruption of glomerular perfusion, and hence of the glomerular filtration rate, thereby constituting a serious threat to systemic homeostasis.

### Ophthalmic Diseases and Disorders

Certain of the compounds of the invention are useful in treating patients suffering from diseases and disorders in which neuroprotection of the optic nerve would be of benefit, for example in:

1. open angle primary/secondary glaucoma
2. multiple sclerosis (optic neuritis)
3. central or brunch retinal vein occlusion
4. ischemic optic neuropathy (in status epilepticus, HIV-1 infection)
5. optic nerve injury
6. tumors extending into the suprasellar region (above the sella turcica)
juxta chiasmal tumors (the visual loss associated with compression of the optic chiasm by pituitary tumors may be transient or permanent, possibly related to the extent of irreversible retrograde degeneration to the retinal ganglion cells.

### Retinoblastoma

### Glaucoma

Glaucoma is one of the leading causes of blindness in the world. It affects approximately 66.8 million people worldwide and at least 12,000 Americans are blinded by this disease each year (Kahn and Milton, Am J Epidemiol. 1980, 111(6):769-76). Glaucoma is characterized by the degeneration of axons in the optic nerve head, primarily due to elevated intraocular pressure (IOP). One of the most common forms of glaucoma, known as primary open-angle glaucoma (POAG), results from the increased resistance of aqueous humor outflow in the trabecular meshwork (TM), causing IOP elevation and eventual optic nerve damage.

### Primary Open-Angle Glaucoma:

The majority of the cases of glaucoma are the form known as primary-open-angle glaucoma POAG, also called chronic open-angle glaucoma). POAG results from a build up of aqueous humor fluid within the anterior chamber of the eye resulting in intraocular pressure (IOP). If too much fluid is entering the eye, or if the trabecular meshwork “drain” gets clogged up (for instance, with debris or cells) so that not enough fluid is leaving the eye, the pressure builds up in what is known as “open angle glaucoma.” Open angle glaucoma also can be caused when the posterior portion of the iris adheres to the anterior surface of the lens creating a “pupillary block”, and preventing intraocular fluid from passing through the pupil into the anterior chamber.

If the angle between and iris and the cornea is too narrow or is even closed, then the fluid backs up, causing increased pressure in what is known as “closed angle glaucoma.”

Untreated glaucoma eventually leads to optic atrophy and blindness.

Normal Tension Glaucoma refers to optic nerve damage when intraocular eye pressure is normal (between 12-22 mmHg). This occurs in about 25-30% glaucoma cases in the US but in Japan, the rates may be as high as 70%. Other factors are present that cause optic nerve damage but do not affect LOP.

Closed-Angle Glaucoma, (also called angle-closure glaucoma) is responsible for 15% of all glaucoma cases. It is less common than POAG in the U.S., but it constitutes about half of the world's glaucoma cases because of its higher prevalence among Asians. The iris is pushed against the lens, sometimes sticking to it, closing off the drainage angle. This can occur very suddenly, resulting in an immediate rise in pressure. It often occurs in genetically susceptible people when the pupil shrinks suddenly. Closed-angle glaucoma can also be chronic and gradual, a less common condition.

Congenital Glaucoma, in which the eye's drainage canals fail to develop correctly, is present from birth. It is very rare, occurring in about 1 in 10,000 newborns. This may be an inherited condition and often can be corrected with microsurgery.

Optic neuritis is an inflammation of the optic nerve that may affect the part of the nerve and disc within the eyeball (papillitis) or the portion behind the eyeball (retrobulbar optic neuritis). Optic neuritis may be caused by any of the following: demyelinating diseases such as multiple sclerosis or post infectious encephalomyelitis; systemic viral or bacterial infections including complications of inflammatory diseases (e.g., sinusitis, meningitis, tuberculosis, syphilis, chorioretinitis, orbital inflammation); nutritional and metabolic diseases (e.g., diabetes, pernicious anemia, hyperthyroidism); Leber's Hereditary Optic Neuropathy, a rare form of inherited optic neuropathy which mainly affects young men; toxins (tobacco, methanol, quinine, arsenic, salicylates, lead); and trauma.

Optic atrophy is a hereditary or acquired loss of vision disorder that results from the degeneration of the optic nerve and optic tract nerve fibers. It may acquired via occlusions of the central retinal vein or artery, arteriosclerotic changes, may be secondary to degenerative retinal disease, may be a result of pressure against the optic nerve, or may be related to metabolic diseases (e.g., diabetes), trauma, glaucoma, or toxicity (to alcohol, tobacco, or other poisons). Degeneration and atrophy of optic nerve fibers is irreversible, although intravenous steroid injections have been seen to slow down the process in some cases.

Papilledema is swelling of the optic disc (papilla), most commonly due to an increase in intracranial pressure (tumor induced), malignant hypertension, or thrombosis of the central retinal vein. The condition usually is bilateral, the nerve head is very elevated and swollen, and pupil response typically is normal. Vision is not affected initially and there is no pain upon eye movement. Secondary optic nerve atrophy and permanent vision loss can occur if the primary cause of the papilledema is left untreated.

Ischemic optic neuropathy is a severely blinding disease resulting from loss of the arterial blood supply to the optic nerve (usually in one eye), as a result of occlusive disorders of the nutrient arteries. Optic neuropathy can be anterior, which causes a pale edema of the optic disc, or posterior, in which the optic disc is not swollen and the abnormality occurs between the eyeball and the optic chiasm. Ischemic anterior optic neuropathy usually causes a loss of vision that may be sudden or occur over several days. Ischemic posterior optic neuropathy is uncommon, and the diagnosis depends largely upon exclusion of other causes, chiefly stroke and brain tumor.

Other diseases and conditions include dry eye, diabetic retinopathy and diabetic macular edema.

### Dry Eye

Dry eye, also known as keratoconjunctivitis sicca or keratitis sicca, is a common problem usually resulting from a decrease in the production of tear film that lubricates the eyes. Most patients with dry eye experience discomfort, and no vision loss; although in severe cases, the cornea may become damaged or infected.

Dry eye is a multifactorial disease of the tears and ocular surface that results in symptoms of discomfort, visual disturbance, and tear film instability with potential damage to the ocular surface. It is accompanied by increased osmolarity of the tear film and inflammation of the ocular surface.

The lacrimal gland is a multilobular tissue composed of acinar, ductal, and myoepithelial cells. The acinar cells account for 80% of the cells present in the lacrimal gland and are the site for synthesis, storage, and secretion of proteins. Several of these proteins have antibacterial (lysozyme, lactoferrin) or growth factor (epidermal growth factor, transforming growth factor α, keratocyte growth factor) properties that are crucial to the health of the ocular surface. The primary function of the ductal cells is to modify the primary fluid secreted by the acinar cells and to secrete water and electrolytes. The myoepithelial cells contain multiple processes, which surround the basal area of the acinar and ductal cells, and are believed to contract and force fluid out of the ducts and onto the ocular surface.

### Mechanisms of Lacrimal Gland Dysfunction

Apoptosis, hormonal imbalance, production of autoantibodies, alterations in signaling molecules, neural dysfunction, and increased levels of proinflammatory cytokines have been proposed as possible mediators of lacrimal gland insufficiency. One of the primary symptoms of Sjögrens Syndrome is dry eye. Apoptosis of the acinar and ductal epithelial cells of the lacrimal glands has been proposed as a possible mechanism responsible for the impairment of secretory function (Manganelli and Fietta, Semin Arthritis Rheum. 2003. 33(1):49-65). Without wishing to be bound by theory, apoptotic epithelial cell death may be due to activation of several apoptotic pathways involving Fas (Apo-1/CD95), FasL (FasL/CD95L), Bax, caspases, perforin, and granzyme B. Cytotoxic T cells through the release of proteases, such as perforin and granzyme B, or the interaction of FasL expressed by T cells with Fas on epithelial cells, can lead to apoptosis of the acinar cells.

The current treatment for dry eye is mainly local and symptomatic such as: tear supplementation with lubricants; tear retention with therapies such as punctal occlusion, moisture chamber spectacles or contact lenses; tear stimulation for example by secretagogues; biological tear substitutes; anti-inflammatory therapy (Cyclosporine, Corticosteroids, Tetracyclines); and dietary essential fatty acids.

In one aspect the present invention provides a method of treating dry-eye in a subject in need thereof, comprising topically administering to the eye of the subject a chemically modified siRNA which inhibits expression of a gene associated with dry-eye. In some embodiments the gene is selected from FAS and FASL. In a currently preferred embodiment the siRNA is formulated for delivery as eye drops. In various embodiments the subject is suffering from Sjögrens syndrome.

### Diabetic Retinopathy

Diabetic retinopathy is a complication of diabetes, secondary to diabetes, and a leading cause of blindness. It occurs when diabetes damages the tiny blood vessels inside the retina. Diabetic retinopathy has four stages:

Mild Nonproliferative Retinopathy: microaneurysms in the retina's blood vessels.

Moderate Nonproliferative Retinopathy. As the disease progresses, some blood vessels that nourish the retina are blocked.

Severe Nonproliferative Retinopathy. Many more blood vessels are blocked, depriving several areas of the retina of a blood supply, which is overcome by the growth of new blood vessels.

Proliferative Retinopathy. The new blood vessels grow along the retina and along the surface of the vitreous gel. When the vessels leak blood, severe vision loss and even blindness can result.

During pregnancy, diabetic retinopathy may be a problem for women with diabetes.

Without wishing to be bound to theory, blood vessels damaged from diabetic retinopathy can cause vision loss in two ways: Fragile, abnormal blood vessels can develop and leak blood into the center of the eye, blurring vision. This is proliferative retinopathy and is the fourth and most advanced stage of the disease. Fluid can leak into the center of the macula, resulting in blurred vision. This condition is called macular edema. It can occur at any stage of diabetic retinopathy, although it is more likely to occur as the disease progresses and is known as diabetic macular edema (DME).

### Age Related Macular Degeneration (AMD)

The most common cause of decreased best-corrected, vision in individuals over 65 years of age in the United States is the retinal disorder known as age-related macular degeneration (AMD). The area of the eye affected by AMD is the macula, a small area in the center of the retina, composed primarily of photoreceptor cells. As AMD progresses, the disease is characterized by loss of sharp, central vision. So-called “dry” AMD accounts for about 85%-90% of AMD patients and involves alterations in eye pigment distribution, loss of photoreceptors and diminished retinal function due to overall atrophy of cells. “Wet” AMD involves proliferation of abnormal choroidal vessels leading to clots or scars in the sub-retinal space. Thus, the onset of “wet” AMD occurs because of the formation of an abnormal choroidal neovascular network (choroidal neovascularization, CNV) beneath the neural retina. The newly formed blood vessels are excessively leaky. This leads to accumulation of subretinal fluid and blood leading to loss of visual acuity. Eventually, there is total loss of functional retina in the involved region, as a large disciform scar involving choroids and retina forms. While dry AMD patients may retain vision of decreased quality, wet AMD often results in blindness. (Hamdi & Kenney, Frontiers in Bioscience, e305-314, May 2003).

### Ocular Ischemic Conditions

Ischemic optic neuropathy (ION) includes a variety of disorders that produce ischemia to the optic nerve. By definition, ION is termed anterior if disc edema is present acutely, suggesting infarction of the portion of the optic nerve closest to the globe. ION also may be posterior, lying several centimeters behind the globe. Ischemic optic neuropathy usually occurs only in people older than 60 years of age. Most cases are nonarteritic and attributed to the effects of atherosclerosis, diabetes, or hypertension on optic nerve perfusion. Temporal arteritis causes about 5% of cases (arteritic ION).

### Ischemic Optic Neuropathy (ION)

A severely blinding disease resulting from loss of the arterial blood supply to the optic nerve (usually in one eye), as a result of occlusive disorders of the nutrient arteries. Optic neuropathy can be anterior (AION), which causes a pale edema of the optic disc, or posterior, in which the optic disc is not swollen and the abnormality occurs between the eyeball and the optic chiasm. Ischemic anterior optic neuropathy usually causes a loss of vision that may be sudden or occur over several days. Ischemic posterior optic neuropathy is uncommon, and the diagnosis depends largely upon exclusion of other causes, chiefly stroke and brain tumor.

### Bone Marrow Transplantation (BMT) Retinopathy

Bone marrow transplantation retinopathy was first reported in 1983. It typically occurs within six months, but it can occur as late as 62 months after BMT. Risk factors such as diabetes and hypertension may facilitate the development of BMT retinopathy by heightening the ischemic microvasculopathy. Patients present with decreased visual acuity and/or visual field deficit. Posterior segment findings are typically bilateral and symmetric. Clinical manifestations include multiple cotton wool spots, telangiectasia, microaneurysms, macular edema, hard exudates and retinal hemorrhages.

Cyclosporine is a powerful immunomodulatory agent that suppresses graft-versus-host immune response. It may lead to endothelial cell injury and neurological side effects, and as a result, it has been suggested as the cause of BMT retinopathy. Total body irradiation has also been implicated as the cause of BMT retinopathy. Radiation injures the retinal microvasculature and leads to ischemic vasculopathy. Chemotherapeutic agents have been suggested as a potential contributing factor in BMT retinopathy. Medications such as cisplatin, carmustine, and cyclophosphamide can cause ocular side effects including papilledema, optic neuritis, visual field deficit and cortical blindness. It has been suggested that these chemotherapeutic drugs may predispose patients to radiation-induced retinal damages and enhance the deleterious effect of radiation. In general, patients with BMT retinopathy have a good prognosis. The retinopathy usually resolves within two to four months after stopping or lowering the dosage of cyclosporine. In one report, 69 percent of patients experienced complete resolution of the retinal findings, and 46 percent of patients fully recovered their baseline visual acuity. Because of the favorable prognosis and relatively non-progressive nature of BMT retinopathy, aggressive intervention is usually not necessary.

### Neurodegenerative Disease

### Spinal Cord Injury

Spinal cord injury or myelopathy, is a disturbance of the spinal cord that results in loss of sensation and/or mobility. The two common types of spinal cord injury are due to trauma and disease. Traumatic injury can be due to automobile accidents, falls, gunshot, diving accidents inter alia, and diseases which can affect the spinal cord include polio, spina bifida, tumors and Friedreich's ataxia.

### Neuropathy

Neuropathy affects all peripheral nerves: pain fibers, motor neurons, autonomic nerves. It therefore necessarily can affect all organs and systems since all are innervated. There are several distinct syndromes based on the organ systems and members affected, but these are by no means exclusive. A patient can have sensorimotor and autonomic neuropathy or any other combination. Despite advances in the understanding of the metabolic causes of neuropathy, treatments aimed at interrupting these pathological processes have been limited by side effects and lack of efficacy. Thus, treatments are symptomatic and do not address the underlying problems. Agents for pain caused by sensorimotor neuropathy include tricyclic antidepressants (TCAs), serotonin reuptake inhibitors (SSRIs) and antiepileptic drugs (AEDs). None of these agents reverse the pathological processes leading to diabetic neuropathy and none alter the relentless course of the illness. Thus, it would be useful to have a pharmaceutical composition that could better treat these conditions and/or alleviate the symptoms.

### Ischemia Reperfusion Injury Following Lung Transplantation

Lung transplantation, the only definitive therapy for many patients with end stage lung disease, has poor survival rates in all solid allograft recipients. Ischemia reperfusion (IR) injury is one of the leading causes of death in lung allograft recipients.

International patent application no. WO 2006/035434 assigned to the assignee of the present invention discloses p53 inhibitors for the treatment of, inter alia, acute renal failure and hearing loss.

### Oral Mucositis

Oral mucositis, also referred to as a stomatitis, is a common and debilitating side effect of chemotherapy and radiotherapy regimens, which manifests itself as erythema and painful ulcerative lesions of the mouth and throat. Routine activities such as eating, drinking, swallowing, and talking may be difficult or impossible for subjects with severe oral mucositis. Palliative therapy includes administration of analgesics and topical rinses.

More effective therapies to treat the above mentioned diseases and disorders would be of great therapeutic value.

The present invention also provides for a process of preparing a pharmaceutical composition, which comprises:
providing one or more double stranded compound of the invention; and
admixing said compound with a pharmaceutically acceptable carrier.

The present invention also provides for a process of preparing a pharmaceutical composition, which comprises admixing one or more compounds of the present invention with a pharmaceutically acceptable carrier.

In a preferred embodiment, the compound used in the preparation of a pharmaceutical composition is admixed with a carrier in a pharmaceutically effective dose. In a particular embodiment the compound of the present invention is conjugated to a steroid or to a lipid or to another suitable molecule e.g. to cholesterol.

### Chemical Modifications

Modifications or analogs of nucleotides can be introduced to improve the therapeutic properties of the nucleotides. Improved properties include increased nuclease resistance and/or increased ability to permeate cell membranes.

Accordingly, the present invention also includes all analogs of, or modifications to, a oligonucleotide of the invention that does not substantially affect the function of the polynucleotide or oligonucleotide. In a preferred embodiment such modification is related to the base moiety of the nucleotide, to the sugar moiety of the nucleotide and/or to the phosphate moiety of the nucleotide.

In one embodiment the modification is a modification of the phosphate moiety, whereby the modified phosphate moiety is selected from the group comprising phosphothioate.

The compounds of the present invention can be synthesized by any of the methods that are well-known in the art for synthesis of ribonucleic (or deoxyribonucleic) oligonucleotides. Such synthesis is, among others, described in Beaucage and Iyer, Tetrahedron 1992; 48:2223-2311; Beaucage and Iyer, Tetrahedron 1993; 49: 6123-6194 and Caruthers, et. al., Methods Enzymol. 1987; 154: 287-313; the synthesis of thioates is, among others, described in Eckstein, Annu. Rev. Biochem. 1985; 54: 367-402, the synthesis of RNA molecules is described in Sproat, in Humana Press 2005 edited by Herdewijn P.; Kap. 2: 17-31 and respective downstream processes are, among others, described in Pingoud et. al., in IRL Press 1989 edited by Oliver R. W. A.; Kap. 7: 183-208.

Other synthetic procedures are known in the art e.g. the procedures as described in Usman et al., 1987, J. Am. Chem. Soc., 109, 7845; Scaringe et al., 1990, NAR., 18, 5433; Wincott et al., 1995, NAR. 23, 2677-2684; and Wincott et al., 1997, Methods Mol. Bio., 74, 59, and these procedures may make use of common nucleic acid protecting and coupling groups, such as dimethoxytrityl at the 5′-end, and phosphoramidites at the 3′-end. The modified (e.g. 2′-O-methylated) nucleotides and unmodified nucleotides are incorporated as desired.

The oligonucleotides of the present invention can be synthesized separately and joined together post-synthetically, for example, by ligation (Moore et al., 1992, Science 256, 9923; Draper et al., International Patent Publication No. WO 93/23569; Shabarova et al., 1991, NAR 19, 4247; Bellon et al., 1997, Nucleosides & Nucleotides, 16, 951; Bellon et al., 1997, Bioconjugate Chem. 8, 204), or by hybridization following synthesis and/or deprotection.

It is noted that a commercially available machine (available, inter alia, from Applied Biosystems) can be used; the oligonucleotides are prepared according to the sequences disclosed herein. Overlapping pairs of chemically synthesized fragments can be ligated using methods well known in the art (e.g., see U.S. Pat. No. 6,121,426). The strands are synthesized separately and then are annealed to each other in the tube. Then, the double-stranded siRNAs are separated from the single-stranded oligonucleotides that were not annealed (e.g. because of the excess of one of them) by HPLC. In relation to the siRNAs or siRNA fragments of the present invention, two or more such sequences can be synthesized and linked together for use in the present invention.

The compounds of the invention can also be synthesized via tandem synthesis methodology, as described for example in US Patent Publication No. US 2004/0019001 (McSwiggen), wherein both siRNA strands are synthesized as a single contiguous oligonucleotide fragment or strand separated by a cleavable linker which is subsequently cleaved to provide separate siRNA fragments or strands that hybridize and permit purification of the siRNA duplex. The linker can be a polynucleotide linker or a non-nucleotide linker.

The present invention further provides for a pharmaceutical composition comprising two or more siRNA molecules for the treatment of any of the diseases and conditions mentioned herein, whereby said two molecules may be physically mixed together in the pharmaceutical composition in amounts which generate equal or otherwise beneficial activity, or may be covalently or non-covalently bound, or joined together by a nucleic acid linker of a length ranging from 2-100, preferably 2-50 or 2-30 nucleotides. In one embodiment, the siRNA molecules are comprised of a double-stranded nucleic acid structure as described herein, wherein the two siRNA sequences are selected from the nucleic acids set forth in Tables A-AQ.

Thus, the siRNA molecules may be covalently or non-covalently bound or joined by a linker to form a tandem siRNA compound. Such tandem siRNA compounds comprising two siRNA sequences are typically of 38-150 nucleotides in length, more preferably 38 or 40-60 nucleotides in length, and longer accordingly if more than two siRNA sequences are included in the tandem molecule. A longer tandem compound comprised of two or more longer sequences which encode siRNA produced via internal cellular processing, e.g., long dsRNAs, is also envisaged, as is a tandem molecule encoding two or more shRNAs. Such tandem molecules are also considered to be a part of the present invention. A tandem compound comprising two or more siRNAs sequences of the invention is envisaged.

siRNA molecules that target the target genes of the invention may be the main active component in a pharmaceutical composition, or may be one active component of a pharmaceutical composition containing two or more siRNAs (or molecules which encode or endogenously produce two or more siRNAs, be it a mixture of molecules or one or more tandem molecules which encode two or more siRNAs), said pharmaceutical composition further being comprised of one or more additional siRNA molecule which targets one or more additional gene. Simultaneous inhibition of said additional gene(s) will likely have an additive or synergistic effect for treatment of the diseases disclosed herein.

Additionally, the siRNA disclosed herein or any nucleic acid molecule comprising or encoding such siRNA can be linked or bound (covalently or non-covalently) to antibodies (including aptamer molecules) against cell surface internalizable molecules expressed on the target cells, in order to achieve enhanced targeting for treatment of the diseases disclosed herein. For example, anti-Fas antibody (preferably a neutralizing antibody) may be combined (covalently or non-covalently) with any siRNA. In another example, an aptamer which can act like a ligand/antibody may be combined (covalently or non-covalently) with any siRNA.

The compounds of the present invention can be delivered either directly or with viral or non-viral vectors. When delivered directly the sequences are generally rendered nuclease resistant. Alternatively the sequences can be incorporated into expression cassettes or constructs such that the sequence is expressed in the cell as discussed herein below. Generally the construct contains the proper regulatory sequence or promoter to allow the sequence to be expressed in the targeted cell. Vectors optionally used for delivery of the compounds of the present invention are commercially available, and may be modified for the purpose of delivery of the compounds of the present invention by methods known to one of skill in the art.

It is also envisaged that a long oligonucleotide (typically 25-500 nucleotides in length) comprising one or more stem and loop structures, where stem regions comprise the sequences of the oligonucleotides of the invention, may be delivered in a carrier, preferably a pharmaceutically acceptable carrier, and may be processed intracellularly by endogenous cellular complexes (e.g. by DROSHA and DICER as described above) to produce one or more smaller double stranded oligonucleotides (siRNAs) which are oligonucleotides of the invention. This oligonucleotide can be termed a tandem shRNA construct. It is envisaged that this long oligonucleotide is a single stranded oligonucleotide comprising one or more stem and loop structures, wherein each stem region comprises a sense and corresponding antisense siRNA sequence of the target genes of the invention. In particular, it is envisaged that this oligonucleotide comprises sense and antisense siRNA sequences as depicted in Tables A-AQ.

All analogues of, or modifications to, a nucleotide/oligonucleotide may be employed with the present invention, provided that said analogue or modification does not substantially affect the function of the nucleotide/oligonucleotide. The nucleotides can be selected from naturally occurring or synthetic modified bases. Naturally occurring bases include adenine, guanine, cytosine, thymine and uracil. Modified bases of nucleotides include inosine, xanthine, hypoxanthine, 2-aminoadenine, 6-methyl, 2-propyl and other alkyl adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine. In some embodiments one or more nucleotides in an oligomer is substituted with inosine.

In addition, analogues of polynucleotides can be prepared wherein the structure of one or more nucleotide is fundamentally altered and better suited as therapeutic or experimental reagents. An example of a nucleotide analogue is a peptide nucleic acid (PNA) wherein the deoxyribose (or ribose) phosphate backbone in DNA (or RNA is replaced with a polyamide backbone which is similar to that found in peptides. PNA analogues have been shown to be resistant to enzymatic degradation and to have extended stability in vivo and in vitro. Other modifications that can be made to oligonucleotides include polymer backbones, cyclic backbones, acyclic backbones, thiophosphate-D-ribose backbones, triester backbones, thioate backbones, 2′-5′ bridged backbone, artificial nucleic acids, morpholino nucleic acids, locked nucleic acid (LNA), glycol nucleic acid (GNA), threose nucleic acid (TNA), arabinoside, and mirror nucleoside (for example, beta-L-deoxynucleoside instead of beta-D-deoxynucleoside). Examples of siRNA compounds comprising LNA nucleotides are disclosed in Elmen et al., (NAR 2005, 33(1):439-447).

The compounds of the present invention can be synthesized using one or more inverted nucleotides, for example inverted thymidine or inverted adenine (see, for example, Takei, et al., 2002, JBC 277(26):23800-06).

The term “unconventional moiety” as used herein refers to abasic ribose moiety, an abasic deoxyribose moiety, a deoxyribonucleotide, a modified deoxyribonucleotide, a mirror nucleotide, a non-base pairing nucleotide analog and a nucleotide joined to an adjacent nucleotide by a 2′-5′ internucleotide phosphate bond; bridged nucleic acids including LNA and ethylene bridged nucleic acids.

The term “capping moiety” as used herein includes abasic ribose moiety, abasic deoxyribose moiety, modifications abasic ribose and abasic deoxyribose moieties including 2′O alkyl modifications; inverted abasic ribose and abasic deoxyribose moieties and modifications thereof; C6-imino-Pi; a mirror nucleotide including L-DNA and L-RNA; 5′OMe nucleotide; and nucleotide analogs including 4′,5′-methylene nucleotide; 1-(β-D-erythrofuranosyl)nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 12-aminododecyl phosphate; hydroxypropyl phosphate; 1,5-anhydrohexitol nucleotide; alpha-nucleotide; threo-pentofuranosyl nucleotide; acyclic 3′,4′-seco nucleotide; 3,4-dihydroxybutyl nucleotide; 3,5-dihydroxypentyl nucleotide, 5′-5′-inverted abasic moiety; 1,4-butanediol phosphate; 5′-amino; and bridging or non bridging methylphosphonate and 5′-mercapto moieties.

Abasic deoxyribose moiety includes for example abasic deoxyribose-3′-phosphate; 1,2-dideoxy-D-ribofuranose-3-phosphate; 1,4-anhydro-2-deoxy-D-ribitol-3-phosphate. Inverted abasic deoxyribose moiety includes inverted deoxyriboabasic; 3′,5′ inverted deoxyriboabasic 5′-phosphate.

A “mirror” nucleotide is a nucleotide with reversed chirality to the naturally occurring or commonly employed nucleotide, i.e., a mirror image (L-nucleotide) of the naturally occurring (D-nucleotide). The nucleotide can be a ribonucleotide or a deoxyribonucleotide and my further comprise at least one sugar, base and or backbone modification. U.S. Pat. No. 6,602,858 discloses nucleic acid catalysts comprising at least one L-nucleotide substitution. Mirror nucleotide includes for example L-DNA (L-deoxyriboadenosine-3′-phosphate (mirror dA); L-deoxyribocytidine-3′-phosphate (mirror dC); L-deoxyriboguanosine-3′-phosphate (mirror dG); L-deoxyribothymidine-3′-phosphate (mirror image dT)) and L-RNA (L-riboadenosine-3′-phosphate (mirror rA); L-ribocytidine-3′-phosphate (mirror rC); L-riboguanosine-3′-phosphate (mirror rG); L-ribouracil-3′-phosphate (mirror dU).

### Screening of Inactivation Compounds for Target Genes:

Some of the compounds and compositions of the present invention may be used in a screening assay for identifying and isolating compounds that modulate the activity of a target gene, in particular compounds that modulate a disorder accompanied by an elevated level of the genes of the invention. The compounds to be screened comprise inter alia substances such as small chemical molecules and antisense oligonucleotides.

The inhibitory activity of the compounds of the present invention on target genes or binding of the compounds of the present invention to target genes may be used to determine the interaction of an additional compound with the target polypeptide, e.g., if the additional compound competes with the oligonucleotides of the present invention for inhibition of a target gene, or if the additional compound rescues said inhibition. The inhibition or activation can be tested by various means, such as, inter alia, assaying for the product of the activity of the target polypeptide or displacement of binding compound from the target polypeptide in radioactive or fluorescent competition assays.

The present invention is illustrated in detail below with reference to examples, but is not to be construed as being limited thereto.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### EXAMPLES

### General Methods in Molecular Biology

Standard molecular biology techniques known in the art and not specifically described were generally followed as in Sambrook et al., Molecular Cloning: A Laboratory Manual, Cold Spring Harbor Laboratory Press, New York (1989), and as in Ausubel et al., Current Protocols in Molecular Biology, John Wiley and Sons, Baltimore, Md. (1989) and as in Perbal, A Practical Guide to Molecular Cloning, John Wiley & Sons, New York (1988), and as in Watson et al., Recombinant DNA, Scientific American Books, New York and in Birren et al (eds) Genome Analysis: A Laboratory Manual Series, Vols. 1-4 Cold Spring Harbor Laboratory Press; New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In situ (In cell) PCR in combination with Flow Cytometry can be used for detection of cells containing specific DNA and mRNA sequences (Testoni et al., 1996, Blood 87:3822.) Methods of performing RT-PCR are also well known in the art.

### Example 1

### Generation of Sequences for Active siRNA Compounds to SOX9, ASPP1, CTSD, CAPNS1, FAS and FAS Ligand Genes and Production of the siRNAs

Using proprietary algorithms and the known sequence of the target genes, the sequences of many potential siRNAs were generated. In addition to the algorithm, some of the 23-mer oligomer sequences were generated by 5′ and/or 3′ extension of the 19-mer sequences. The sequences that have been generated using this method are fully complementary to the corresponding mRNA sequence.

Tables A-AQ provide siRNAs for the following target genes: SOX9, ASPP1, CTSD, CAPNS1, FAS and FAS ligand. For each gene there is a separate list of 19-mer, 21-mer and 23-mer siRNA sequences, which are prioritized based on their score in the proprietary algorithm as the best sequences for targeting the human gene expression.

The following abbreviations are used in the Tables A-AQ herein: “other spec or Sp.” refers to cross species identity with other animals: RB—Rabbit, CHMP—Chimpanzee, GP—Guinea-Pig, MS—Mouse; ORF: open reading frame. 19-mers, 21-mers and 23-mers refer to oligomers of 19, 21 and 23 ribonucleic acids in length.

In Vitro Testing of the siRNA Compounds for the Target Genes

### 1. General

About 1.5-2×10⁵ test cells (HEPG2 or PC3 cells for siRNA targeting the human gene) were seeded per well in 6 wells plate (70-80% confluent).

After 24 h cells were transfected with siRNA oligomers using Lipofectamine™ 2000 reagent (Invitrogene) at final concentration of 500 pM, 5 nM, 20 nM or 40 nM. The cells were incubated at 37° C. in a CO2 incubator for 72 h.

As positive control for cells transfection PTEN-Cy3 labeled siRNA oligos was used. As negative control for siRNA activity GFP siRNA oligos were used.

About 72 h after transfection cells were harvested and RNA was extracted from cells. Transfection efficiency was tested by fluorescent microscopy.

The percent of inhibition of gene expression using specific siRNAs was determined using qPCR analysis of target gene in cells expressing the endogenous gene. Other siRNA according to Tables A-AQ are tested in vitro where it is shown that these siRNA compounds inhibit gene expression.

TABLE 2
Percent of knockdown of the expression of Human ASPP1, Human
SOX9 in cells using chemically modified 19-mer siRNA molecules.

| | | | | % of |
|---|---|---|---|---|
| Table/ | Cell | | | control** |
| cmpd # | line* | siRNA | Sequence (5′ > 3′) | (inhibition) |
| Table E | HEPG2 | ASPP1_1 | Sense: GCAAAAUCAUGAACGGCAA | 100, 49 |
| #7 | cells | | AS: UUGCCGUUCAUGAUUUUGC | |
| Table E | HEPG2 | ASPP1_2 | Sense: CGAACUCAGAGAAAUGUAA | 48, 48, 43 |
| #17 | cells | | AS: UUACAUUUCUCUGAGUUCG | |
| Table E | HEPG2 | ASPP1_3 | Sense: CCUUUGAUCAUAUGAUGUA | 72, 74 |
| #306 | cells | | AS: UACAUCAUAUGAUCAAAGG | |
| Table E | HEPG2 | ASPP1_4 | Sense: GGAGAAAAACGUACUGAAA | 57 (5 nM), 38 |
| #385 | cells | | AS: UUUCAGUACGUUUUUCUCC | |
| Table E | HEPG2 | ASPP1_5 | Sense: GACAAGUCGACUACAGCAA | 64, 100 |
| #215 | cells | | AS: UUGCUGUAGUCGACUUGUC | |
| Table A | PC3 | SOX9_1 | Sense: CCGAGAUGAUCCUAAAAAU | 40, 28, 58 |
| #2 | cells | | AS: AUUUUUAGGAUCAUCUCGG | |
| Table A | PC3 | SOX9_2 | Sense: GCCGAGAUGAUCCUAAAAA | 64, 45, 57 |
| #81 | cells | | AS: UUUUUAGGAUCAUCUCGGC | |
| Table A | PC3 | SOX9_3 | Sense: CCUUCAACCUCCCACACUA | 58, 41, 23 |
| #5 | cells | | AS: UAGUGUGGGAGGUUGAAGG | |
| Table A | PC3 | SOX9_4 | Sense: CUCGUACCCAAAUUUCCAA | 81, 57, 100 |
| #29 | cells | | AS: UUGGAAAUUUGGGUACGAG | |
| Table A | PC3 | SOX9_5 | Sense: CCUUCAUGAAGAUGACCGA | 65, 46, 64 |
| #50 | cells | | AS: UCGGUCAUCUUCAUGAAGG | |

* cell line used in assay

TABLE 3
knockdown of expression of mouse rat and human CAPNS 1

| | | | | KD (in % of control)** | | |
|---|---|---|---|---|---|---|
| Table | siRNA | Sense seq (5′ > 3′) | | | RAT | |
| cmpd # | ID | AS sequence (5′ > 3′) | species | HUM | (PC12) | Mouse |
| | CAPNS1_7 | CCUCUGGAACAACAUUAAA | ms, | | 79, 19, | 46, 37, 34 |
| | | UUUAAUGUUGUUCCAGAGG | rat | | 18 | (50 nM) |
| | CAPNS1_8 | GCUAGCUUGUUCUACUGAA | ms | | | 37,30,22 |
| | | UUCAGUAGAACAAGCUAGC | | | | (50 nM) |
| | CAPNS1_9 | GGAACAUGGAUUUCGAUAA | ms, rat | | 3, 56, 22 | 40, 24, 29 |
| | | UUAUCGAAAUCCAUGUUCC | | | | (50 nM) |
| | CAPNS1_{—1}0 | GGAGGGAACAUGAGCUCUA | ms | | | 73,73 |
| | | UAGAGCUCAUGUUCCCUCC | | | | |
| | CAPNS1_11 | CGUAGUCAUUACUCCAAUA | ms | | | 48,68 |
| | | UAUUGGAGUAAUGACUACG | | | | |
| | CAPNS1_12 | CCACGUAGCCAUUACUCUA | rat | | 12, 32, 2 | 43, 53 |
| | | UAGAGUAAUGGCUACGUGG | | | | |
| | CAPNS1_13 | GCUACAGAACUCAUGAAUA | rat | | 7, 43, 3 | 37, 37, 36 |
| | | UAUUCAUGAGUUCUGUAGC | | | | (50 nM) |
| Table I | CAPNS1_14_{—} | GCGCCACAGAACUCAUG [symbol] A | Hu, | | | |
| #6 | S129 | UUCAUGAGUUCUGUGGCGC | ms, mn | | | |
| Table I | CAPNS1_14_{—} | GCGCCACAGAACUCAUG [symbol] A | Hu, | | | |
| #6 | S505 | UUCAUGAGUUCUGUGGCGC | ms, mn | | | |
| Table I | CAPNS1_15_{—} | CUGACUAUGUAUUCCUG [symbol] A | Hu, | | | |
| #13 | S129 | UUCAGGAAUACAUAGUCAG | ms, mn | | | |
| Table I | CAPNS1_15_{—} | CUGACUAUGUAUUCCUG [symbol] A | Hu, | | | |
| #13 | S505 | UUCAGGAAUACAUAGUCAG | ms, mn | | | |
| Table I | CAPNS1_16_{—} | GCUGACUAUGUAUUCCU [symbol] A | Hu, ms, | | | |
| #55 | S129 | UCAGGAAUACAUAGUCAGC | mn, rat | | | |
| Table I | CAPNS1_16_{—} | GCUGACUAUGUAUUCCU [symbol] A | Hu, ms, | | | |
| #55 | S505 | UCAGGAAUACAUAGUCAGC | mn, rat | | | |
| Table I | CAPNS1_17_{—} | UGGGCUUUGAGGAAUUC [symbol] A | Hu, mn | | | |
| #36 | S129 | UUGAAUUCCUCAAAGCCCA | | | | |
| Table I | CAPNS1_17_{—} | UGGGCUUUGAGGAAUUC [symbol] A | Hu, mn | | | |
| #36 | S505 | UUGAAUUCCUCAAAGCCCA | | | | |
| Table I | CAPNS1_18_{—} | GGCUUUGAGGAAUUCAA [symbol] U | Hu, mn | | | |
| #61 | S129 | ACUUGAAUUCCUCAAAGCC | | | | |
| Table I | CAPNS1_18_{—} | GGCUUUGAGGAAUUCAA [symbol] U | Hu, mn | | | |
| #61 | S505 | ACUUGAAUUCCUCAAAGCC | | | | |

Legend Table 3: ** % of control in separate tests using 20nM concentration of siRNA
molecules (unless indicated otherwise). All sequences are presented in a 5′-3′ orientation.
cross species specificity: mn: Macaca mulatta and Chimpanzee, hu: human; ms:mouse

The above-mentioned SOX9_{—}1 to SOX9_{—}5, ASPP1_{—}1 to ASPP1_{—}5 and CAPNS1_{—}7-CAPNS_{—}13 siRNA compounds were synthesized with single alternating unmodified and 2′OMe modified ribonucleotides on both strands. The sense strands comprise unmodified ribonucleotides at the 3′ and 5′ termini, and the antisense strands comprising 2′OMe modified ribonucleotides at the 3′ and 5; termini. The CAPNS1_{—}14 to CAPNS1_{—}18 siRNA comprise sense strands comprising an L-DNA moiety at position 18 (bold, italicized and underlined) and antisense strands with variant alternating patterns of unmodified and 2′OMe modified (underlined) ribonucleotides. A preferred structure for the siRNA according to the present invention is an siRNA wherein (N)x comprises 2′O Me modified ribonucleotides at positions 2, 4, 6, 8, 11, 13, 15, 17, 19 and (N′)y comprises at least one L-deoxyribonucleotide at the 3′ penultimate position.

### Example 2

### Model Systems of Acute Renal Failure (ARF)

ARF is a clinical syndrome characterized by rapid deterioration of renal function that occurs within days. Without being bound by theory the acute kidney injury may be the result of renal ischemia-reperfusion injury such as renal ischemia-reperfusion injury in patients undergoing major surgery such as major cardiac surgery. The principal feature of ARF is an abrupt decline in glomerular filtration rate (GFR), resulting in the retention of nitrogenous wastes (urea, creatinine). Recent studies, support that apoptosis in renal tissues is prominent in most human cases of ARF. The principal site of apoptotic cell death is the distal nephron. During the initial phase of ischemic injury, loss of integrity of the actin cytoskeleton leads to flattening of the epithelium, with loss of the brush border, loss of focal cell contacts, and subsequent disengagement of the cell from the underlying substratum.

Testing an active siRNA compound is performed using an animal model for ischemia-reperfusion-induced ARF. Ischemia-reperfusion injury is induced in rats following 45 minutes bilateral kidney arterial clamp and subsequent release of the clamp to allow 24 hours of reperfusion. siRNA compounds are injected into the jugular vein of individual experimental animals 30 minutes prior to and 4 hours following the clamp. ARF progression is monitored by measurement of serum creatinine levels before (baseline) and 24 hrs post surgery. At the end of the experiment, the rats are perfused via an indwelling femoral line with warm PBS followed by 4% paraformaldehyde. The left kidneys are surgically removed and stored in 4% paraformaldehyde for subsequent histological analysis. Acute renal failure is frequently defined as an acute increase of the serum creatinine level from baseline. An increase of at least 0.5 mg per dL or 44.2 μmol per L of serum creatinine is considered as an indication for acute renal failure. Serum creatinine is measured at time zero before the surgery and at 24 hours post ARF surgery.

SiRNA molecules according to Tables A-AQ are tested in this animal model which show that these siRNA compounds treat and/or prevent ARF.

### Example 3

### Model Systems of Spinal Cord Injury

Spinal cord injury, or myelopathy, is a disturbance of the spinal cord that results in loss of sensation and/or mobility. The two common types of spinal cord injury are due to trauma and disease. Traumatic injury can be due to automobile accidents, falls, gunshot, diving accidents inter alia, and diseases which can affect the spinal cord include polio, spina bifida, tumors and Friedreich's ataxia.

Uptake of siRNA Molecules into Neurons Following Injection into Injured Spinal-Cord:

The uptake of Cy3 labeled siRNA (delivered by injection into the injured cord) in different types of cells was examined following spinal cord contusion in 18 rats and in uninjured rats (9 rats). Sagittal cryosections were produced and immunostaining using four different groups of antibodies was performed in order to determine whether uptake has occurred in neurons, astroglia, oligodendroglia and/or macrophages/microglia. Markers for neurons were NeuN, or GAP43; markers for astroglia and potential neural stem cells were GFAP, nestin or vimentin; markers for oligodendroglia were NG2 or APC; markers for macrophages/microglia were ED1 or Iba-1 (Hasegawa et al., 2005. Exp Neurol 193 394-410).

Six rats were injected with two different doses of Cy3 labeled siRNA (1 μg/μl, 10 μg/μl) and were left for 1 and 3 days before sacrifice. Histological analyses indicate that many long filamentous profiles have taken up the labeled siRNA as well as other processes and cell bodies. Immunostaining with antibodies to MAP2 has identified uptake of label into dendrites and into cell bodies of neurons including motor neurons. Staining with other antibodies specific to astrocytes or macrophages revealed lower uptake of Cy3 labeled siRNA as compared to neurons. These results indicate that siRNA molecules injected to the injured spinal-cord reach the cell body and dendrites of neurons including motor neurons.

### The Spinal-Cord Injury Animal Model:

Adult female Sprague-Dawley rats are anesthetized with 40 mg/kg of pentobarbital and the spinal thoracic T9-10 is exposed by laminectomy. Contusive injury are produced by dropping a 10 gm rod onto the exposed spinal cord from a height of 12.5 mm using MASCIS (Multicenter Animal Spinal Cord Injury Study) impactor (as described In Basso et al., Journal of Neurotrauma Vol 12 (1), p 1-21 1995 and in Basso et al., Journal of Neurotrauma Vol 13 (7), p 343-59 1996). Prior to injury, three point injections of the tested siRNA are performed at the injury epicenter 2 mm rostral and caudal to the epicenter. GFP siRNA is injected in additional five rats as a control. Each injection is conducted slowly during a period of 10 min into dorsal column (˜1 mm depth) of T10 using a Hamilton syringe. Following injections, muscles and skin are closed separately. The behavioral assessment of the recovery following the spinal cord contusion is preformed using an open field locomotor test as described by Basso et al (the BBB locomotor rating scale).

Instead of using direct injection to the spinal cord, it is possible to deliver the siRNA molecules to the spinal-cord by using intrathecal delivery (such as using Alzet pump).

siRNA according to Tables A-AQ, in particular to the SOX9, CTSD, CAPNS1 genes are tested in this animal model which show that these siRNA compounds treat and/or prevent spinal-cord injury.

### Example 4

### Model Systems of Hearing Loss Conditions

### (i) Knock Down of Target Gene in Cochlea of Chemotherapeutic Treated Animals

The aim of this study is to evaluate siRNA-induced knockdown of target gene mRNA or protein expression level in rat inner ear tissues when siRNA delivered by eardrops or transtympanic injection in cisplatin-treated rats.

In previous experiments in which cisplatin treated rats were treated with siRNA compounds, daily body weight loss and increased levels of serum creatinine at the termination day were shown to correspond to cisplatin induced nephro- and ototoxicity.

### Experimental Design

The experiment includes the following experimental groups: CAPNS1 siRNA treated (15 rats/group) and untreated control group (12 rats) as described below. CAPNS1 siRNA is tested with various chemical modifications including siRNA shown in Table 3.

Experimental group I: is treated with single siRNA administration: at a dose of 100 μg/vehicle/10 μl, delivered by transtympanic injection or eardrops. siRNA treatment is performed on day 1 (study initiation), prior to the 1^{st} Cisplatin administration. The contralateral, Left ear serves as untreated control. Cisplatin is injected (i.p.) at daily dose of 2 mg/kg for five consecutive days; injection volume: 0.6 ml for 300 g BW (stock concentration: 50 mg/ml; LD50 I.P. dose 6.4 mg/kg). Termination step will be −3 days (72 hrs) after the last Cisplatin administration (Day 8 after study initiation)

Experimental group II: is treated with two doses of siRNA in the same dose regime and delivery route used for group I. 1^{st} siRNA treatment is given on day 1 (study initiation) immediately after the 1^{st} Cisplatin administration, 2^{nd} siRNA treatment is given on Day 8 (after study initiation). During the second week of the study Cisplatin is administered i.p. in the same dose and design regimes as done during the 1^{st} experimental week, 4 additional Cisplatin injections (Days 8-11). Termination step is performed 24 hrs after the last Cisplatin administration on Day 12 after study initiation.

Experimental Group III (untreated normal control rats) are sacrificed on Days 9 or 11.

### For All Groups:

Anesthesia: Rats are anesthetized with 4 ml/kg body weight (BW) of Equithesine (i.p.).

Right ear: A 10 μl sample volume eardrops is slowly instilled into the external REAC (Right external auditory canal), using blunt pipette tip. This volume is delivered into the right ear of all rats from groups I and II according study design. During and after REAC instillations, rats are kept on the contra lateral side for one hour, and are returned to cage after regaining consciousness. Alternatively a 10 μl sample volume eardrops is injected transtympanically to the right ear.

Cisplatin Administration: Rats (experimental groups I and II) are subjected to consecutive i.p. injections of Cisplatin according to the experimental design at a daily dose of: 2 mg/kg (i.p.); (injection volume: 0.6 ml per 300 g BW.).

Body weight is recorded daily for all experimental groups. Serum samples are collected from experimental groups I-II) for serum Creatinine (CREA) baseline/termination levels (all groups).

Clinical signs are recorded 30 minutes after single i.p. administration (for all cisplatin injected animals). Observations include: morbidity/mortality, autonomic activity (lacrimation, salivation piloerection), changes in gait, posture and response to handling as well as the presence of unusual behavior, tremors and convulsions etc.

Scheduled euthanasia: Rats from groups I-III are euthanized at 24 hrs or 72 hrs after last Cisplatin injection in order to assess target gene mRNA and protein time course accumulation and its knock down by siRNA treatment.

### Termination Step: by Cardiac Puncture and Blood Collection

### Tissue Collection:

### Inner Ear Soft Tissues (Modiolus Connected to it Soft Tissue and Base of the Cochlear Nerve):

All rats from all groups are decapitated. Left and right temporal bones including cochlea are gently harvested from all animals; modiolus connected to soft tissue and the base of the cochlear nerve is dissected on ice, snap frozen in liquid nitrogen, analyzed for target mRNA (qPCR) and protein (ELISA).

Kidneys (left and right) are snap frozen in liquid nitrogen in labeled 50 ml round bottom tubes and analyzed for target mRNA and protein.

### Serum/Whole Blood is Analyzed for Serum Creatinine (CREA)

(ii) Distribution of Cy3-PTEN siRNA in the Cochlea Following Local Application to the Round Window of the Ear:

A solution of 1 μg/100 μl of Cy3-PTEN siRNA (total of 0.3-0.4 μg) PBS was applied to the round window of chinchillas. The Cy3-labelled cells within the treated cochlea were analyzed 24-48 hours post siRNA round window application after sacrifice of the chinchillas. The pattern of labeling within the cochlea was similar following 24 h and 48 h and includes labeling in the basal turn of cochlea, in the middle turn of cochlea and in the apical turn of cochlea. Application of Cy3-PTEN siRNA onto scala tympani revealed labelling mainly in the basal turn of the cochlea and the middle turn of the cochlea. The Cy3 signal was persistence to up to 15 days after the application of the Cy3-PTEN siRNA. These results indicated for the first time that local application of siRNA molecules within the round window led to significant penetration of the siRNA molecules to the basal, middle and apical turns of the cochlea. The siRNA compounds of the invention are tested in this animal model which shows that there is significant penetration of these siRNA compounds to the basal, middle and apical turns of the cochlea, and that these compounds may be used in the treatment of hearing loss.

(iii) Chinchilla Model of Carboplatin-Induced or Cisplatin-Induced Cochlea Hair Cell Death

Chinchillas are pre-treated by direct administration of specific siRNA in saline to the left ear of each animal. Saline is given to the right ear of each animal as placebo. Two days following the administration of the specific siRNA compounds of the invention, the animals are treated with carboplatin (75 mg/kg ip) or cisplatin (intraperitoneal infusion of 13 mg/kg over 30 minutes). After sacrifice of the chinchillas (two weeks post carboplatin treatment) the % of dead cells of inner hair cells (THC) and outer hair cells (OHC) is calculated in the left ear (siRNA treated) and in the right ear (saline treated). It is calculated that the % of dead cells of inner hair cells (IHC) and outer hair cells (OHC) is lower in the left ear (siRNA treated) than in the right ear (saline treated).

### (iv) Chinchilla Model of Acoustic-Induced Cochlea Hair Cell Death:

The activity of specific siRNA in an acoustic trauma model is studied in chinchilla. The animals are exposed to an octave band of noise centered at 4 kHz for 2.5 h at 105 dB. The left ear of the noise-exposed chinchillas is pre-treated (48 h before the acoustic trauma) with 30 μg of siRNA in ˜10 μL of saline; the right ear is pre-treated with vehicle (saline). The compound action potential (CAP) is a convenient and reliable electrophysiological method for measuring the neural activity transmitted from the cochlea. The CAP is recorded by placing an electrode near the base of the cochlea in order to detect the local field potential that is generated when a sound stimulus, such as click or tone burst, is abruptly turned on. The functional status of each ear is assessed 2.5 weeks after the acoustic trauma. Specifically, the mean threshold of the compound action potential recorded from the round window is determined 2.5 weeks after the acoustic trauma in order to determine if the thresholds in the siRNA-treated ear are lower (better) than the untreated (saline) ear. In addition, the amount of inner and outer hair cell loss is determined in the siRNA-treated and the control ear.

siRNA molecules according to Tables A-AQ are tested in this animal model which shows that the thresholds in the siRNA-treated ear are lower (better) than in the untreated (saline) ear. In addition, the amount of inner and outer hair cell loss is lower in the siRNA-treated ear than in the control ear.

### Example 5

### Model Systems of Dry Eye

### Mouse and Rat Models for Lacrimal Inflammation (Dacryoadenitis):

1. The nonobese diabetic (NOD) mouse model. Male NOD mice show significant inflammatory lesions of the lacrimal gland from the age of 8 weeks.

2. The MRL/MpJ-fas_/fas_ (MRL/_) and MRL/MpJ-faslprl/faslpr (MRL/lpr) mouse models of Sjo\ddot{}gren's syndrome exhibit lacrimal gland infiltrates characterized by a predominance of CD4 T cells. The extent of the lacrimal gland inflammation is significantly greater in lacrimal glands of female MRL/_ and MRL/lpr mice.

3. The IQI/Jic has recently been established as a new mouse model for primary Sjo\ddot{}gren's syndrome. The lymphocytic infiltration is well restricted to salivary and lacrimal glands.

4. Rat Model: Experimental immune dacryoadenitis may be produced also in Lewis rats by sensitization with a single intradermal administration of an extract of lacrimal gland in complete Freund's adjuvant (CFA) and simultaneous intravenous injection of killed Bordetella pertussis.

### Models for Evaporative Dry Eye:

The tear film is constantly exposed to multiple environmental factors, including variable temperatures, airflow, and humidity, which may stimulate or retard its evaporation. The lipids produced by the Meibomian glands and spread onto the aqueous phase by the shear forces produced by each blink, protect the tear film from excessive evaporation. Short-term models for hyperevaporative dry eye have been created by preventing rabbits from blinking through placement of lid specula or sutures. After 2 hours of desiccation induced by lid specula, dry spots appear on the rabbit corneal epithelial surface and stain with methylene blue.

siRNA according to Tables A-AQ, in particular to the FAS and FAS ligand genes are tested in this animal model which show that these siRNA compounds treat and/or prevent dry eye.

### Example 6

### Model Systems of Glaucoma and Ischemic Optic Neuropathy

Testing the active siRNA of the invention for treating or preventing glaucoma is preformed in rat animal model for optic nerve crush described for example in: Maeda, K. et al., “A Novel Neuroprotectant against Retinal Ganglion Cell Damage in a Glaucoma Model and an Optic Nerve Crush Model in the Rat”, Investigative Ophthalmology and visual Science (IOVS), March 2004, 45(3)851. Specifically, for optic nerve transection the orbital optic nerve (ON) of anesthetized rats is exposed through a supraorbital approach, the meninges severed and all axons in the ON transected by crushing with forceps for 10 seconds, 2 mm from the lamina cribrosa.

siRNA compounds of the invention and in particular, siRNA to ASPP1, CTSD and CAPNS1, are tested in this animal model and the results show that these siRNA compounds are useful in treating and/or preventing glaucoma.

Other animal models for glaucoma and ischemic optic neuropathy for testing the siRNA compounds of the invention are as following:

For optic ischemia reperfusion model: Tsujikawa A. et al., “In Vivo Evaluation of Leukocyte Dynamics in Retinal Ischemia Reperfusion Injury”, Invest Ophthal and Visual Science (IOVS), April 1998, 39(5):793-800.

For elevated intraocular pressure model: Morrison, J C et al., “A rat model of chronic pressure-induced optic nerve damage”. Exp Eye Res. 1997. 64, 85-96.

For pharmacological ischemia reperfusion model: Masuzawa, K. et al., “A Model of Retinal Ischemia-Reperfusion Injury in Rats by Subconjunctival Injection of Endothelin-1”. Exp. Biol. Med. 2006. 231(6):1085-9.

### Example 6A

### Optic Nerve Axotomy Model

The purpose of the present study was to use a model of Retinal Ganglion Cell (RGC) apoptosis induced by axotomy of the optic nerve (ON) in adult Sprague-Dawley rats. The onset and kinetics of RGC death in this model system are reproducible and allow for the establishment of the neuroprotective efficacy of siRNA in vivo. Using this method, the time course of RGC death follows a predictable course: cell death begins on day 5 and proceeds to the rapid loss of more than 90% of these neurons by 2 weeks.

### Methods

Retrograde labeling of RGCs: For the purpose of this study, RGCs were labeled by application of the retrograde tracer FluoroGold (2%, Fluorochrome, Englewood, Colo.) in the superior colliculus. Briefly, both superior colliculi were exposed and a small piece of gelfoam soaked in FluoroGold was applied to their surface. In adult rats, the time required to obtain full labeling of all RGCs following this procedure is ˜1 week. For this reason, optic nerve axotomy and intraocular injection of siRNA molecules were performed one week after retrograde labeling of RGCs.

Optic nerve axotomy: The entire population of RGCs were axotomized by transecting the optic nerve close to the eye (0.5 to 1 mm). Retinal fundus examination was routinely performed after each axotomy to check the integrity of the retinal circulation after surgery. Animals showing signs of compromised blood supply were excluded from the study.

For intravitreal injection 10 μg in 5 μl of PBS each of the reagents, either ASPP1 siRNA or GFP siRNA were microinjected into the vitreous body 2 mm anterior to the nerve head, perpendicular to the sclera, using a glass micropipette at the time of surgery, day 0, and then repeated at day 7.

Quantification of RGC survival: Experimental and control animals were perfused transcardially with 4% paraformaldehyde at 14 days after optic nerve axotomy. The left retinas (treated) and the right retinas (untreated controls) were dissected out, fixed for an additional 30 min and flat-mounted vitreal side up on a glass slide for examination of the ganglion cell layer. RGCs backfilled with FluoroGold were counted in 12 standard retinal areas. Microglia that may have incorporated FluoroGold after phagocytosis of dying RGCs were distinguished by their characteristic morphology and excluded from our quantitative analyses.

### Experimental Design

Test article: ASPP1, CTSD, CAPNS1 siRNA: a double-stranded 19-mer oligonucleotide stabilized by 2′O-methylation on the antisense strand and L-DNA on the sense strand.

### Control Articles:

### PBS

siRNA targeting GFP—a double-stranded 21-mer oligonucleotide stabilized by 2′O-methylation on both strands.

CNL—siRNA with no match to any known mammalian transcript; a double-stranded 19-mer oligonucleotide stabilized by 2′O-methylation on both strands.

Overview of the experimental protocol: RGCs were selectively labeled first by application of the retrograde tracer FluoroGold to the superior colliculus. One week later, animals were subjected to optic nerve transection concomitantly with intravitreal injections of siRNA. The second intravitreal injection was performed on day 7 of the experiment. The quantification of surviving RGCs was carried out at 14 days after optic nerve axotomy by counting FluoroGold-labeled RGCs on flat-mounted retinas. Table 4 shows an example of groups' treatments.

TABLE 4

| | | Time of | Time of | Number of | |
|---|---|---|---|---|---|
| | SiRNA | administration | analysis | animals per | Sample |
| siRNA | Dose | (post axotomy) | (post axotomy) | group | preparation |
| siGFP, | 10 ug × 2 | Time 0 | 2 weeks | 8 | Flat mounts |
| axotomy (OS) | | 1 week | | | |
| Test siRNA | 10 ug × 2 | Time 0 | 2 weeks | 8 | Flat mounts |
| Axotomy (OS) | | 1 week | | | |

The siRNA compounds of the invention are tested in this animal model and the results show that these siRNA compounds are useful in effecting neuroprotection of the retinal ganglion.

### Example 6B

### Rat Optic Nerve Crush (ONC) Model: Intravitreal siRNA Delivery and Eye Drop Delivery

For optic nerve transection the orbital optic nerve (ON) of anesthetized rats is exposed through a supraorbital approach, the meninges severed and all axons in the ON transected by crushing with forceps for 10 seconds, 2 mm from the lamina cribrosa.

The siRNA compounds are delivered alone or in combination in 5 uL volume (10 ug/uL) as eye drops. Immediately after optic nerve crush (ONC), 20 ug/10 ul test siRNA or 10 ul PBS is administered to one or both eyes of adult Wistar rats and the levels of siRNA taken up into the dissected and snap frozen whole retinae at 5 h and 1 d, and later at 2 d, 4 d, 7 d, 14 d and 21 d post injection is determined. Similar experiments are performed in order to test activity and efficacy of siRNA administered via eye drops.

### Example 7

### Model Systems of Ischemia Reperfusion Injury Following Lung Transplantation in Rats

Lung ischemia/reperfusion injury is achieved in a rat animal model as described in Teruaki Mizobuchi et al., The Journal of Heart and Lung Transplantation, Vol 23 No. 7 (2004) and in Kazuhiro Yasufuku et al., Am. J. Respir. Cell Mol Biol, Vol 25, pp 26-34 (2001).

Specifically, after inducing anesthesia with isoflurane, the trachea is cannulated with a 14-gauge Teflon catheter and the rat is mechanically ventilated with rodent ventilator using 100% oxygen, at a rate of 70 breaths per minute and 2 cm H₂O of positive end-respiratory pressure. The left pulmonary artery, veins and main stem bronchus are occluded with a Castaneda clamp. During the operation, the lung is kept moist with saline and the incision is covered to minimize evaporative losses. The period of ischemia is 60 minutes long. At the end of the ischemic period the clamp is removed and the lung is allowed to ventilate and reperfuse for further 4 h, 24 h, and 5 d post induction of lung ischemia. At the end of the experiment, the lungs are gently harvested and either frozen for RNA extraction or fixed in glutaraldehyde cocktail for subsequent histological analysis.

The siRNA compounds of the invention are tested in this animal model and the results show that these siRNA compounds are useful in treating and/or preventing ischemia reperfusion injury following lung transplantation.

### Example 8

### Model Systems for Acute Lung Injury (ALI)

Intratracheal (i.t) administration of LPS (Lipopolysaccharide), a bacterial cell wall component, is an accepted experimental model of acute lung injury (ALI), as LPS stimulates profound lung recruitment of inflammatory cells and the subsequent development of systemic inflammation.

(See, for example, Fang W F, et al., Am J Physiol Lung Cell Mol Physiol. 2007 293(2):L336-44; Hagiwara S, Iwasaka H, Noguchi T. J Anesth. 2007; 21(2):164-70).

The siRNA compounds of the invention are tested in this animal model and the results show that these siRNA compounds are useful in treating and/or preventing acute lung injury.

### Example 9

### Model Systems of Acute Respiratory Distress Syndrome

The active siRNA compounds of the invention are tested in an animal model for acute respiratory distress syndrome as described, for example, by Chen, et al. J Biomed Sci. 2003; 10(6 Pt 1):588-92).

The results show that the siRNA compounds according to the present invention are useful in treating and/or preventing acute respiratory distress syndrome.

### Example 10

### Model Systems of Chronic Obstructive Pulmonary Disease (COPD)

Chronic obstructive pulmonary disease (COPD) is characterized mainly by emphysema, which is permanent destruction of peripheral air spaces, distal to terminal bronchioles. Emphysema is also characterized by accumulation of inflammatory cells such as macrophages and neutrophils in bronchioles and alveolar structures. Emphysema and chronic bronchitis may occur as part of COPD or independently and involve apoptosis.

Testing the active inhibitors of the invention (such as siRNA) for treating COPD/emphysema/chronic bronchitis is done in the following animal models:

Cigarette smoke-induced emphysema model: chronic exposure to cigarette smoke causes emphysema in several animals such as, inter alia, mouse, guinea pig.

Lung protease activity as a trigger of emphysema.

VEGFR inhibition model of emphysema.

Bronchial instillation with human neutrophil/pancreatic elastase in rodents.

MMP (matrix metalloprotease)-induced emphysema.

Inflammation-induced emphysema.

These models and others are described in co-assigned PCT patent application WO 2006/023544, and PCT/IL2008/000522 which are hereby incorporated by reference into this application. The siRNA compounds of the invention prevent formation of emphysema.

### Example 11

### Model Systems for Transplantation-Associated Acute Kidney Injury

Warm ischemia—A left nephrectomy is performed, followed by auto transplantation that resulted in a warm kidney graft preservation period of 45 minutes. Following auto transplantation, a right nephrectomy is performed on the same animal. An siRNA compound is administered intravenously via the femoral vein either before harvesting of the kidney graft (mimicking donor treatment) (“pre”), or after the kidney autotransplantation (mimicking recipient treatment), or both before harvest and after transplantation (combined donor and recipient treatment) (“pre-post”).

Cold ischemia—A left nephrectomy is performed on a donor animal, followed by a cold preservation (on ice) of the harvested kidney for a period of 5 hours. At the end of this period, the recipient rat undergoes a bilateral nephrectomy, followed by transplantation of the cold-preserved kidney graft. The total warm ischemia time (including surgical procedure) is 30 minutes. SiRNA is administered intravenously via the femoral vein, either to the donor animal prior to the kidney harvest (“pre”), or to the recipient animal 15 minutes (“post 15 min”) or 4 hours (post 4 hrs) post-transplantation.

To assess the efficacy of the siRNA compound in improvement of post-transplantation renal function, serum creatinine levels are measured on days 1, 2, and 7 post-transplantation in both warm and cold ischemia models.

### Example 12

### Model Systems for Neurodegenerative Diseases and Disorders

### Example 12A

### Evaluating the Efficacy of Intranasal Administration of siRNA Compounds in the APP Transgenic Mouse Model of Alzheimer's Disease

Animals and Treatment. The study includes twenty-four (24) APP [V717I] transgenic mice (female), a model for Alzheimer's disease (Moechars D. et al., EMBO J. 15(6):1265-74, 1996; Moechars D. et al., Neuroscience. 91(3):819-30), aged 11 months that are randomly divided into two equal groups (Group I and Group II).

Animals are treated with intranasal administration of SOX9, ASPP1, CTSD, CAPNS1, FAS and FAS ligand siRNA (200-400 μg/mice, Group I) and vehicle (Group II), 2-3 times a week, during 3 months.

Termination. Mice are sacrificed; brains are dissected and process one hemisphere for histology and freeze one hemisphere for shipment.

Evaluation. The following histological analysis is performed:

1. Anti-Aβ staining and quantification (4 slides/mouse):
2. Thio S staining and quantification (4 slides/mouse):
3. CD45 staining and quantification (4 slides/mouse):
4. GFAP (astrocytosis) staining and quantification:

### Example 12B

### Evaluating the Efficacy of Intranasal Administration of Specific siRNAs in a BACE-Transgenic Mouse Model of Alzheimer's Disease

Objective. The objective of this study is to test the efficacy of intranasal delivery of specific siRNA in BACE-transgenic mouse model for Alzheimer disease.

Animals and Treatment. The study includes twenty (20) BACE-1 transgenic mice (female/male), aged 4 months that are randomly divided into two equal groups. siRNA treatment is initiated at age 4 months. siRNA is administered intranasally.

### Evaluation.

1. Behavioral test. All animals are monitored and tested for behavioral changes by subjecting the animals to periodical behavioral analysis. Spatial learning and memory in the Morris water maze is used.

2. Brain biochemistry. The brains of five (5) mice in each group are subjected to biochemical analysis. Western blot analysis of BACE, APP, CTFs and Aβ is carried out. Assay for BACE enzymatic activity is performed.

3. Immunohistochemistry. The left hemibrain of five (5) mice in each group is subjected to immunohistochemical analysis. Expression levels of BACE, APP and CTF are determined.

4. Analysis of gene knockdown by qPCR are performed in the right hemibrain of five (5) mice in each group

### Example 12C

### Evaluating the Efficacy of Intranasal Administration of siRNA in a Mouse Model of ALS

Objective. To examine the efficacy of siRNA directed to the SOX9, ASPP1, CTSD, CAPNS1, FAS and FAS ligand genes in the mutant SOD1^{G93A} mouse model of ALS.

Animals and Treatment. The following experimental groups are used for studying disease progression and lifespan:

1. Group 1—Mismatch siRNA—wild-type (n=10) and SOD1^{G93A} mice (n=10)
2. Group 2—SOX9, ASPP1, CTSD, CAPNS1, FAS and FAS ligand siRNA—wild-type (n=10) and SOD1^{G93A} mice (n=10)
3. Group 3-Untreated controls—wild-type (n=10) and SOD1^{G93A} mice (n=10)

Each experimental group is sex matched (5 male, 5 female) and contain littermates from at least 3 different litters. This design reduces bias that may be introduced by using mice from only a small number of litters, or groups of mice with a larger percentage of female SOD1^{G93A} mice (since these mice live 3-4 days longer than males).

Administration of siRNA. The route of administration of the siRNA is intranasal, with administration twice weekly, starting from 30 days of age.

Analysis of disease progression. Behavioral and electromyography (EMG) analysis in treated and untreated mice is performed to monitor disease onset and progression. Mice are pre-tested before start of siRNA treatment, followed by weekly assessments. All results are compared statistically. The following tests are performed:

1. Swimming tank test: this test is particularly sensitive at detecting changes in hind-limb motor function (Raoul et al., 2005. Nat Med. 11, 423-428; Towne et al, 2008. Mol Ther. 16: 1018-1025).

2. Electromyography: EMG assessments are performed in the gastrocnemius muscle of the hind limbs, where compound muscle action potential (CMAP) is recorded (Raoul et al., 2005. supra).

3. Body weight: The body weight of mice is recorded weekly, as there is a significant reduction in the body weight of SOD1^{G93A} mice during disease progression (Kieran et al., 2007. PNAS USA. 104, 20606-20611).

Assessment of lifespan. The lifespan in days for treated and untreated mice is recorded and compared statistically to determine whether siRNA treatment has any significant effect on lifespan. Mice are sacrificed at a well-defined disease end point, when they have lost >20% of body weight and are unable to raise themselves in under 20 seconds. All results are compared statistically.

Post mortem histopathology. At the disease end-point mice are terminally anaesthetized and spinal cord and hind-limb muscle tissue are collected for histological and biochemical analysis.

Examining motor neuron survival. Transverse sections of lumbar spinal cord are cut using a cryostat and stained with gallocyanin, a nissl stain. From these sections the number of motor neurons in the lumbar spinal cord is counted (Kieran et al., 2007. supra), to determine whether siRNA treatment prevents motor neuron degeneration in SOD1^{G93A} mice.

Examining spinal cord histopathology. Motor neuron degeneration in SOD1^{G93A} mice results in astrogliosis and activation of microglial cells. Here, using transverse sections of lumbar spinal cord the activation of astocytes and microglial cells is examined using immunocytochemistry to determine whether siRNA treatment reduced or prevented their activation.

Examining muscle histology. Hind-limb muscle denervation and atrophy occur as a consequence of motor neuron degeneration in SOD1^{G93A} mice. At the disease end point the weight of individual hind-limb muscles (gastrocnemius, soleus, tibialis anterior, extensor digitorium longus muscles) is recorded and compared between treated and untreated mice.

Muscles are then processed histologically to examine motor end plate denervation and muscle atrophy (Kieran et al., 2005. J Cell Biol. 169, 561-567).

For further elaboration on model systems which are used to test the compounds of the present invention, see International patent publication Nos. WO 06/023544A2, WO 2006/035434 and WO 2007/084684A2, co-assigned or assigned to the assignee of the present invention, which are hereby incorporated by reference in their entirety.

Lengthy table referenced here
US20110034534A1-20110210-T00001
Please refer to the end of the specification for access instructions.

Lengthy table referenced here
US20110034534A1-20110210-T00002
Please refer to the end of the specification for access instructions.

Lengthy table referenced here
US20110034534A1-20110210-T00003
Please refer to the end of the specification for access instructions.

Lengthy table referenced here
US20110034534A1-20110210-T00004
Please refer to the end of the specification for access instructions.

Lengthy table referenced here
US20110034534A1-20110210-T00005
Please refer to the end of the specification for access instructions.

Lengthy table referenced here
US20110034534A1-20110210-T00006
Please refer to the end of the specification for access instructions.

Lengthy table referenced here
US20110034534A1-20110210-T00007
Please refer to the end of the specification for access instructions.

Lengthy table referenced here
US20110034534A1-20110210-T00008
Please refer to the end of the specification for access instructions.

Lengthy table referenced here
US20110034534A1-20110210-T00009
Please refer to the end of the specification for access instructions.

Lengthy table referenced here
US20110034534A1-20110210-T00010
Please refer to the end of the specification for access instructions.

Lengthy table referenced here
US20110034534A1-20110210-T00011
Please refer to the end of the specification for access instructions.

Lengthy table referenced here
US20110034534A1-20110210-T00012
Please refer to the end of the specification for access instructions.

Lengthy table referenced here
US20110034534A1-20110210-T00013
Please refer to the end of the specification for access instructions.

Lengthy table referenced here
US20110034534A1-20110210-T00014
Please refer to the end of the specification for access instructions.

Lengthy table referenced here
US20110034534A1-20110210-T00015
Please refer to the end of the specification for access instructions.

Lengthy table referenced here
US20110034534A1-20110210-T00016
Please refer to the end of the specification for access instructions.

Lengthy table referenced here
US20110034534A1-20110210-T00017
Please refer to the end of the specification for access instructions.

Lengthy table referenced here
US20110034534A1-20110210-T00018
Please refer to the end of the specification for access instructions.

Lengthy table referenced here
US20110034534A1-20110210-T00019
Please refer to the end of the specification for access instructions.

Lengthy table referenced here
US20110034534A1-20110210-T00020
Please refer to the end of the specification for access instructions.

Lengthy table referenced here
US20110034534A1-20110210-T00021
Please refer to the end of the specification for access instructions.

Lengthy table referenced here
US20110034534A1-20110210-T00022
Please refer to the end of the specification for access instructions.

Lengthy table referenced here
US20110034534A1-20110210-T00023
Please refer to the end of the specification for access instructions.

Lengthy table referenced here
US20110034534A1-20110210-T00024
Please refer to the end of the specification for access instructions.

Lengthy table referenced here
US20110034534A1-20110210-T00025
Please refer to the end of the specification for access instructions.

Lengthy table referenced here
US20110034534A1-20110210-T00026
Please refer to the end of the specification for access instructions.

Lengthy table referenced here
US20110034534A1-20110210-T00027
Please refer to the end of the specification for access instructions.

Lengthy table referenced here
US20110034534A1-20110210-T00028
Please refer to the end of the specification for access instructions.

Lengthy table referenced here
US20110034534A1-20110210-T00029
Please refer to the end of the specification for access instructions.

Lengthy table referenced here
US20110034534A1-20110210-T00030
Please refer to the end of the specification for access instructions.

Lengthy table referenced here
US20110034534A1-20110210-T00031
Please refer to the end of the specification for access instructions.

Lengthy table referenced here
US20110034534A1-20110210-T00032
Please refer to the end of the specification for access instructions.

Lengthy table referenced here
US20110034534A1-20110210-T00033
Please refer to the end of the specification for access instructions.

Lengthy table referenced here
US20110034534A1-20110210-T00034
Please refer to the end of the specification for access instructions.

Lengthy table referenced here
US20110034534A1-20110210-T00035
Please refer to the end of the specification for access instructions.

Lengthy table referenced here
US20110034534A1-20110210-T00036
Please refer to the end of the specification for access instructions.

Lengthy table referenced here
US20110034534A1-20110210-T00037
Please refer to the end of the specification for access instructions.

Lengthy table referenced here
US20110034534A1-20110210-T00038
Please refer to the end of the specification for access instructions.

Lengthy table referenced here
US20110034534A1-20110210-T00039
Please refer to the end of the specification for access instructions.

Lengthy table referenced here
US20110034534A1-20110210-T00040
Please refer to the end of the specification for access instructions.

Lengthy table referenced here
US20110034534A1-20110210-T00041
Please refer to the end of the specification for access instructions.

Lengthy table referenced here
US20110034534A1-20110210-T00042
Please refer to the end of the specification for access instructions.

Lengthy table referenced here
US20110034534A1-20110210-T00043
Please refer to the end of the specification for access instructions.

Lengthy table referenced here
US20110034534A1-20110210-T00044
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20110034534A1). An electronic copy of the table will also be available from the USPTO upon request and payment of the fee set forth in 37 CFR 1.19(b)(3).

1. A compound having the structure:
5′(N)ₓ—Z3′ (antisense strand)
3′Z′—(N′)_{y}-z″5′ (sense strand)
wherein each of N and N′ is a ribonucleotide which may be unmodified or modified, or an unconventional moiety;
wherein each of (N)x and (N′)y is an oligonucleotide in which each consecutive N or N′ is joined to the next N or N′ by a covalent bond;
wherein Z and Z′ may be present or absent, but if present is independently 1-5 consecutive nucleotides covalently attached at the 3′ terminus of the strand in which it is present;
wherein z″ may be present or absent, but if present is a capping moiety covalently attached at the 5′ terminus of (N′)y;
each of x and y is independently an integer between 18 and 40;
wherein the sequence of (N′)y is substantially complementary to the sequence of (N)x; and
wherein (N)x comprises an antisense sequence present in any of Tables A-AQ.

2. The compound of claim 1, wherein the covalent bond joining each consecutive N or N′ is a phosphodiester bond.

3. The compound of claim 1, wherein x=y.

4. The compound of claim 3, wherein each of x and y is 19, 21 or 23.

5. The compound of claim 1, wherein Z and Z′ are absent.

6. The compound of claim 1, wherein one of Z or Z′ is present.

7. The compound of claim 1, wherein each of N or N′ is unmodified in its sugar residue.

8. The compound of claim 1, wherein at least one N or N′ comprises a modification in its sugar residue.

9. The compound of claim 8, wherein the modification comprises a modification at the 2′ position.

10. The compound of claim 9, wherein the modification at the 2′ position comprises the presence of an amino, a fluoro, an alkoxy or an alkyl group.

11. The compound of claim 10 wherein the modification comprises the presence of an alkoxy group.

12. The compound of claim 11, wherein the alkoxy group is methoxy (2′-O-methyl) group.

13. The compound of claim 1, wherein alternating ribonucleotides in (N)ₓ are 2′-O-methyl modified and alternating ribonucleotides in (N′)_{y} are 2′-O-methyl modified.

14. The compound of claim 13, wherein each N at the 5′ and 3′ termini in (N)ₓ are modified in their sugar residues, and each N′ at the 5′ and 3′ termini of (N′)_{y} are unmodified in their sugar residues.

15. The compound of claim 14, wherein both (N)ₓ and the (N′)_{y} are non-phosphorylated at both their 3′ and 5′ termini or wherein both (N)ₓ and (N′)_{y} are phosphorylated at the 3′ termini.

16. The compound according to claim 1 wherein (N′)y comprises at least one unconventional moiety.

17. The compound according to claim 16 wherein the unconventional moiety is selected from a mirror nucleotide and a nucleotide joined to an adjacent nucleotide by a 2′-5′ internucleotide phosphate bond.

18. The compound according to claim 17 wherein (N)x comprises 2′O Methyl modified ribonucleotides at positions 2, 4, 6, 8, 11, 13, 15, 17, 19 and (N′)y comprises at least one L-deoxyribonucleotide at the 3′ penultimate position.

19. A pharmaceutical composition comprising a compound of claims 1 or a vector capable of expressing such a compound in an amount effective to inhibit the gene; and a pharmaceutically acceptable carrier.

20. A method of treating a subject suffering from a disease or condition selected from a neurodegenerative disease or disorder including spinal cord injury, Alzheimer's disease (AD) and Amyotrophic Lateral Sclerosis (ALS); acute renal failure (ARF); hearing loss; an ophthalmic disease including glaucoma and ischemic optic neuropathy (ION); a respiratory disease including acute respiratory distress syndrome (ARDS), chronic obstructive pulmonary disease (COPD) and other acute lung and respiratory injuries; injury (e.g. ischemia-reperfusion injury) in organ transplant including lung, kidney, bone marrow, heart, pancreas, cornea or liver transplantation; nephrotoxicity, pressure sores, dry eye syndrome or oral mucositis, comprising administering to the subject an siRNA compound which inhibits expression of a gene selected from SOX9, ASPP1, CTSD, CAPNS1, FAS and FAS ligand in an amount effective to treat the disease or condition.

21. The method according to claim 20 wherein the siRNA comprises an oligomer whose sequence is present in any one of Tables A-AQ.

22. A method of treating a subject suffering from a disease or condition selected from the group consisting of spinal cord injury, Alzheimer's disease (AD) and Amyotrophic Lateral Sclerosis (ALS) comprising administering to the subject an siRNA which inhibits expression of a gene selected from SOX9, ASPP1, CTSD, CAPNS1, in an amount effective to treat the disease or condition.

23. A method of treating a subject suffering from an ophthalmic disease or condition selected from the group consisting of glaucoma, ION and an eye disease secondary to diabetes comprising administering to the subject an siRNA which inhibits expression of a gene selected from SOX9, ASPP1, CTSD, CAPNS1, in an amount effective to treat the disease or condition.

24. A method of treating a subject suffering from dry eye syndrome comprising administering to the subject an siRNA which inhibits expression of a gene selected from FAS and FAS ligand in an amount effective to treat the disease or condition.

25. A compound according to any one of claim 1 for use in therapy for treating a patient in need of neuroprotection.

26. A compound according to claim 25, wherein the patient is in need of optic nerve neuroprotection.

27. A compound according to claim 1 formulated for intranasal administration, for intratracheal administration, or for topical non-invasive administration as an eye drop or ear drop.
